(19) United States
(12) Patent Application Publication
(10) Pub. No.: US 2025/0001252 A1
(43) Pub. Date: Jan. 2, 2025

(54) BREATHING EXERCISE METHODS

(71) Applicant: Berendo Scientific, LLC, Los Angeles, CA (US)

(72) Inventors: Lorig Sima Kalaydjian, Los Angeles, CA (US); Benjamin Scott Arnett, Morgan Hill, CA (US); Nick Hulme, Morgan Hill, CA (US); Jeffrey J. Christian, Morgan Hill, CA (US); Seth Heneveld, Morgan Hill, CA (US); Minhvy Tran, Morgan Hill, CA (US); Spenser Burke, Morgan Hill, CA (US); Dale M. Carlson, JR., Fridley, MN (US); Shahn S. Sage, Fridley, MN (US); Dheeraj Varma Kalidindi, Fridley, MN (US); Elliot W. Bridgeman, Fridley, MN (US); Andrew Richardson, Fridley, MN (US); Mark A. McCarthy, Fridley, MN (US); Eric J. Kezirian, Los Angeles, CA (US)

(21) Appl. No.: 18/345,341

(22) Filed: Jun. 30, 2023

(51) Int. Cl. A63B 23/18; A61M 16/20

(52) U.S. Cl. CPC A63B 23/185; A61M 16/208

(57) ABSTRACT

Methods are disclosed in which a user performs breathing exercises with breathing apparatuses. The breathing apparatus may include a member operatively arranged with a valve mechanism with a threshold pressure. The valve mechanism of the breathing apparatus may be configured to respond when the absolute pressure within the member is greater than a threshold pressure. The breathing apparatus may be configured such that the threshold pressure of the valve mechanism is modifiable.

### BACKGROUND

Sleep disordered breathing, including snoring and obstructive sleep apnea, affects tens of millions of adults in the United States. It is associated with substantial cardiovascular morbidity and mortality, endocrine disturbances, excessive daytime sleepiness, quality of life and performance deficits, and motor vehicle crashes.

Multiple factors contribute to sleep disordered breathing, including the decrease in muscle tone that occurs with sleep onset. The breathing passages of the upper airway, including the nose, oral cavity, and pharynx, are surrounded by muscles and other soft tissues of the head and neck. This decrease in muscle tone enables collapse or vibration of these soft tissues, particularly in the oral cavity and pharynx, contributing to sleep disordered breathing. Treatment of sleep disordered breathing includes approaches that directly or indirectly enlarge or stabilize the breathing passages of the upper airway. Treatment options include behavioral measures such as weight loss, positive airway pressure therapy, surgery, and oral appliances. All treatments have strengths and weaknesses, whether limited compliance, risks of complications, or outcomes that can vary widely.

Head and neck exercises have been proposed as an alternative treatment for sleep disordered breathing. These isotonic and/or isometric exercises can involve muscles within and surrounding the oral cavity and/or pharynx. A number of the exercises that have been proposed may include the generation of positive or negative pressure within the oral cavity and/or pharynx. A number of the exercises may include movement of the tongue.

### SUMMARY

A method for performing a breathing exercise includes positioning an open portion of a member to a user's nose such that breathing by the user through the open portion creates a continuous air pathway from the nose to lungs of the user. The method also includes performing the breathing exercise in a manner such that the user repeatedly generates a pressure within the open portion that exceeds a threshold pressure of a valve mechanism to cause the valve mechanism biased to a closed position to open.

A method for performing a breathing exercise includes positioning an open portion of a member to a user's nose such that breathing by the user through the open portion creates a continuous air pathway from the nose to lungs of the user. The method also includes performing the breathing exercise in a manner such that the user repeatedly generates a pressure within the open portion that exceeds a threshold pressure of a valve mechanism to cause the valve mechanism biased to an open position to close.

### BRIEF DESCRIPTION OF THE DRAWINGS

FIG. 1A is a perspective view of a head and neck exercise apparatus.

FIG. 1B is an exploded view of the head and neck exercise apparatus in FIG. 1A.

FIG. 1C is a cut-away view of the head and neck exercise apparatus in FIG. 1A.

FIG. 1D is a perspective view with hidden lines of the head and neck exercise apparatus in FIG. 1A.

FIG. 1E is a perspective view of the valve plate assembly of the head and neck exercise apparatus in FIG. 1A.

FIG. 1F is an exploded view of the valve plate assembly in FIG. 1D.

FIG. 1G is a frontal view of the valve plate assembly in FIG. 1D.

FIG. 1H is a cross-sectional view of the valve plate assembly in FIG. 1D.

FIG. 1I is a cross-sectional view of a portion of the valve plate assembly in FIG. 1D.

FIG. 2A is an exploded view of a head and neck exercise apparatus.

FIG. 2B is a perspective view of the head and neck exercise apparatus in FIG. 2A.

FIG. 2C is an exploded view of another head and neck exercise apparatus.

FIG. 2D is a perspective view of the another head and neck exercise apparatus in FIG. 2C.

FIG. 2E is an exploded view of a different another head and neck exercise apparatus.

FIG. 2F is a perspective view of the different another head and neck exercise apparatus in FIG. 2E.

FIG. 3A is a perspective view of a head and neck exercise apparatus.

FIG. 3B is an exploded view of the head and neck exercise apparatus in FIG. 3A.

FIG. 3C is a perspective view of the valve plate assembly of the head and neck exercise apparatus in FIG. 3A.

FIG. 3D is an exploded view of the valve plate assembly of the head and neck exercise apparatus in FIG. 3A.

FIG. 3E is a frontal view of the valve plate assembly in FIG. 3C.

FIG. 3F is a cross-sectional view of the valve plate assembly in FIG. 3C.

FIG. 4A is a perspective view of a head and neck exercise apparatus.

FIG. 4B is a perspective view with hidden lines of the head and neck exercise apparatus in FIG. 4A.

FIG. 4C is an exploded view of the head and neck exercise apparatus in FIG. 4A.

FIG. 4D is a perspective view of the valve plate assembly of the head and neck exercise apparatus in FIG. 4A.

FIG. 4E is an exploded view of the valve plate assembly of the head and neck exercise apparatus in FIG. 4A.

FIG. 5A is an exploded view of a head and neck exercise apparatus.

FIG. 5B is a cut-away view of the head and neck exercise apparatus in FIG. 5A.

FIG. 5C is a perspective view of the valve plate assembly of the head and neck exercise apparatus in FIG. 5A.

FIG. 5D is an exploded view of the valve plate assembly in FIG. 5D.

FIG. 6A is an exploded view of the head and neck exercise apparatus in FIG. 6A.

FIG. 6B is a cut-away view of the head and neck exercise apparatus in FIG. 6A.

FIG. 6C is a perspective view of the valve plate assembly of the head and neck exercise apparatus in FIG. 6A.

FIG. 6D is an exploded view of the valve plate assembly in FIG. 6C.

FIG. 7A is an exploded view of a head and neck exercise apparatus.

FIG. 7B is an outline view of the head and neck exercise apparatus in FIG. 7A.

FIG. 7C is a perspective view of the valve plate assembly of the head and neck exercise apparatus in FIG. 7A.

FIG. 7D is an exploded view of the valve plate assembly of the head and neck exercise apparatus in FIG. 7A.

FIG. 7E is a frontal view of the valve plate assembly in FIG. 7C.

FIG. 7F is a cross-sectional view of the valve plate assembly in FIG. 7C.

FIG. 7G is a cross-sectional view of a portion of the valve plate assembly in FIG. 7C.

FIG. 8A is an exploded view of a head and neck exercise apparatus.

FIG. 8B is a perspective view of the valve plate assembly of the head and neck exercise apparatus in FIG. 8A.

FIG. 8C is an exploded view of the valve plate assembly in FIG. 8B.

FIG. 8D is a frontal view of the valve plate assembly in FIG. 8B.

FIG. 8E is a cross-sectional view of the valve plate assembly in FIG. 8B.

FIG. 9A is a perspective view of a head and neck exercise apparatus.

FIG. 9B is a cut-away view of the head and neck exercise apparatus in FIG. 9A.

FIG. 9C is an exploded view of the head and neck exercise apparatus in FIG. 9A.

FIG. 9D is a perspective view of a valve assembly of the head and neck exercise apparatus in FIG. 9A.

FIG. 9E is an exploded view of the valve assembly in FIG. 9D.

FIG. 9F is a side view of the valve assembly in FIG. 9D.

FIG. 9G is an end view of the valve assembly in FIG. 9D.

FIG. 9H is a cross-sectional view of the valve assembly in FIG. 9D.

FIG. 9I is a side view of the valve assembly in FIG. 9D with both valves open.

FIG. 9J is a cross-sectional view of the valve assembly in FIG. 9D with both valves open.

FIG. 9K is a side view of the valve assembly in FIG. 9D with 1 valve closed.

FIG. 9L is a cross-sectional view of the valve assembly in FIG. 9D with 1 valve closed.

FIG. 9M is a side view of the valve assembly in FIG. 9D with 1 valve closed.

FIG. 9N is a cross-sectional view of the valve assembly in FIG. 9D with 1 valve closed.

FIG. 10A is a perspective view of a valve assembly of a head and neck exercise apparatus.

FIG. 10B is a perspective view of the valve assembly in FIG. 10A.

FIG. 10C is a cross-sectional view of the valve assembly in FIG. 10A with the valve open.

FIG. 10D is a cross-sectional view of the valve assembly in FIG. 10A with the valve closed.

FIG. 11A is a perspective view of a head and neck exercise apparatus.

FIG. 11B is an exploded view of the head and neck exercise apparatus in FIG. 11A.

FIG. 11C is an end-on view of the head and neck exercise apparatus in FIG. 11A.

FIG. 11D is a cross-sectional side view of the head and neck exercise apparatus in FIG. 11A.

FIG. 12A is an exploded view of a head and neck exercise apparatus.

FIG. 12B is an end view of the head and neck exercise apparatus in FIG. 12A.

FIG. 12C is a cross-sectional side view of the head and neck exercise apparatus in FIG. 12A.

FIG. 13A is a perspective view of a head and neck exercise apparatus.

FIG. 13B is an exploded view of the head and neck exercise apparatus in FIG. 13A.

FIG. 13C is a cut-away view of the head and neck exercise apparatus in FIG. 13A.

FIG. 13D is a perspective view of a valve plate adjustment mechanism of the head and neck exercise apparatus in FIG. 13A.

FIG. 14A is an exploded view of a head and neck exercise apparatus.

FIG. 14B is a cut-away view of the head and neck exercise apparatus in FIG. 14A.

FIG. 15A is an exploded view of a head and neck exercise apparatus.

FIG. 15B is a cut-away view of the head and neck exercise apparatus in FIG. 15A.

FIG. 16A is a perspective view of a head and neck exercise apparatus.

FIG. 16B is a cut-away view of the head and neck exercise apparatus in FIG. 16A.

FIG. 16C is an exploded view of the head and neck exercise apparatus in FIG. 16A.

FIG. 16D is a cross-sectional view of a valve mechanism in place in the head and neck exercise apparatus in FIG. 16A in the closed (left) and open (right) positions.

FIG. 17A is an exploded view of a head and neck exercise apparatus.

FIG. 17B is a cut-away view of the head and neck exercise apparatus in FIG. 17A.

FIG. 18A is an exploded view of a head and neck exercise apparatus.

FIG. 18B is a cut-away view of the head and neck exercise apparatus in FIG. 18A.

FIG. 19A is a schematic diagram of a mechanism for configuring a portion of a head and neck exercise apparatus.

FIG. 19B is a schematic diagram of another mechanism for configuring a portion of a head and neck exercise apparatus.

FIG. 20A is an exploded view of a head and neck exercise apparatus.

FIG. 20B is a cut-away view of the head and neck exercise apparatus in FIG. 12A.

FIG. 20C is a perspective view of the head and neck exercise apparatus in FIG. 20A.

FIG. 20D is a perspective view of the head and neck exercise apparatus with a similar design to the head and neck exercise apparatus in FIG. 20A.

FIG. 20E is a perspective view of the head and neck exercise apparatus with a similar design to the head and neck exercise apparatus in FIG. 20A.

FIG. 20F is a perspective view of the head and neck exercise apparatus with a similar design to the head and neck exercise apparatus in FIG. 20A.

FIG. 21 is a perspective view of a head and neck exercise apparatus.

FIG. 22 is a perspective view of a head and neck exercise apparatus.

FIG. 23A is a perspective view of a head and neck exercise apparatus.

FIG. 23B is an exploded view of the head and neck exercise apparatus in FIG. 23A.

FIG. 23C is a cut-away view of the head and neck exercise apparatus in FIG. 23A.

FIG. 23D is an assembly view of the head and neck exercise apparatus in FIG. 23A.

FIG. 23E is an end view of the head and neck exercise apparatus in FIG. 23A.

FIG. 23F is another end view of the head and neck exercise apparatus in FIG. 23A.

FIG. 23G is another end view of the head and neck exercise apparatus in FIG. 23A.

FIG. 24 is an exploded view of a head and neck exercise apparatus.

FIG. 25 is an exploded view of a head and neck exercise apparatus.

FIG. 26 is a schematic diagram of a head and neck exercise apparatus.

FIG. 27A is a perspective view of a head and neck exercise apparatus.

FIG. 27B is another perspective view of the head and neck exercise apparatus in FIG. 27A.

FIG. 27C is a side view of the head and neck exercise apparatus in FIG. 27A.

FIG. 28A is a perspective view of a head and neck exercise apparatus.

FIG. 28B is an exploded view of the head and neck exercise apparatus in FIG. 28A.

FIG. 28C is another exploded view of the head and neck exercise apparatus in FIG. 28A.

FIG. 29A is a perspective view of a head and neck exercise apparatus.

FIG. 29B is an exploded view of the head and neck exercise apparatus in FIG. 29A.

FIG. 29C is a perspective view of another head and neck exercise apparatus.

FIG. 29D is an exploded view of the another head and neck exercise apparatus in FIG. 29C.

FIG. 29E is the inner valve member of the head and neck exercise apparatus in FIGS. 29A and 29C.

FIG. 29F is an end view of the inner valve member of the head and neck exercise apparatus in FIG. 29E.

FIG. 29G is a cross-sectional view of the inner valve member of the head and neck exercise apparatus in FIG. 29E.

FIG. 29H is a perspective view with hidden lines of a portion of the head and neck exercise apparatus in FIGS. 29A and 29C.

FIG. 29I is a perspective view with hidden lines of the inner valve member of the head and neck exercise apparatus in FIG. 29E.

FIG. 30A is a perspective view of a head and neck exercise apparatus.

FIG. 30B is an exploded view of the mask interface and head and neck exercise apparatus in FIG. 30A.

FIG. 30C is a perspective view of another head and neck exercise apparatus.

FIG. 30D is an exploded view of the mask interface and head and neck exercise apparatus in FIG. 30C.

FIG. 30E is a perspective view of the head and neck exercise apparatus in FIG. 30A.

FIG. 30F is a cut-away view of the head and neck exercise apparatus in FIG. 30A.

FIG. 30G is an exploded view of the head and neck exercise apparatus in FIG. 30A.

FIG. 30H is a perspective view of the rotating element and motor of the head and neck exercise apparatus in FIG. 30A.

FIG. 30I is an end view of the rotating element and motor of the head and neck exercise apparatus in FIG. 30A.

FIG. 31A is a perspective view of a head and neck exercise apparatus.

FIG. 31B is a cross-sectional view of the head and neck exercise apparatus in FIG. 18A.

FIG. 32A is a perspective view of a head and neck exercise apparatus.

FIG. 32B is a top view of the head and neck exercise apparatus in FIG. 32A.

FIG. 32C is a side view of the head and neck exercise apparatus in FIG. 32A.

FIG. 32D is a cross-sectional view of the head and neck exercise apparatus in FIG. 32A.

FIG. 33A is a perspective view of a head and neck exercise apparatus.

FIG. 33B is another perspective view of the head and neck exercise apparatus in FIG. 33A.

FIG. 33C is a front view of the head and neck exercise apparatus in FIG. 33A.

FIG. 33D is a back view of the head and neck exercise apparatus in FIG. 33A.

FIG. 33E is an exploded view and perspective view of the head and neck exercise apparatus in FIG. 33A.

FIG. 33F is three views of the head and neck exercise apparatus in FIG. 33A: open position with moveable element outside apparatus (left), open position with moveable element inside apparatus (middle), and closed position with moveable element inside apparatus (right).

FIG. 33G is a front view of the moveable element for the head and neck exercise apparatus in FIG. 33A.

FIG. 33H is a cross-sectional view of the moveable element for the head and neck exercise apparatus in FIG. 33A.

FIG. 34A is a perspective view of a head and neck exercise apparatus.

FIG. 34B is a front view of the head and neck exercise apparatus in FIG. 34A.

FIG. 34C is a top view of the head and neck exercise apparatus in FIG. 34A.

FIG. 34D is an exploded view and perspective view of the head and neck exercise apparatus in FIG. 34A.

FIG. 35A is a perspective view of a head and neck exercise apparatus.

FIG. 35B is perspective views of the moveable element of the head and neck exercise apparatus in FIG. 35A.

FIG. 36A is a front view of a head and neck exercise apparatus.

FIG. 36B is front perspective views of the head and neck exercise apparatus in FIG. 36A with lateral securing mechanism in an open (left) and secure (right) position.

FIG. 36C is a back perspective view of the head and neck exercise apparatus in FIG. 36A.

FIG. 37 is a front view of a head and neck exercise apparatus.

FIG. 38A is a perspective view of a head and neck exercise apparatus.

FIG. 38B is a front view of a housing of the head and neck exercise apparatus in FIG. 38A.

FIG. 38C is a front view of a framed moveable element of the head and neck exercise apparatus in FIG. 38A.

FIG. 39A is a perspective view of a head and neck exercise apparatus.

FIG. 39B is a frontal view of the head and neck exercise apparatus in FIG. 39A.

FIG. 39C is a top view of the head and neck exercise apparatus in FIG. 39A.

FIG. 39D is an exploded view of the head and neck exercise apparatus in FIG. 39A.

FIG. 39E is frontal views of the head and neck exercise apparatus in FIG. 39A at different settings.

FIG. 40A is a perspective view of a head and neck exercise apparatus.

FIG. 40B is a top view of the head and neck exercise apparatus in FIG. 40A.

FIG. 40C is a cross-sectional view of the head and neck exercise apparatus in FIG. 40A.

FIG. 40D is an exploded view and perspective view of the head and neck exercise apparatus in FIG. 40A.

FIG. 41 is a flowchart of a method for instructing a user to perform a head and neck exercise.

FIG. 42 is a flowchart of a method for instructing a user to perform a head and neck exercise.

FIG. 43 is a flowchart of a method for instructing a user to perform a combination of head and neck exercises.

FIG. 44 is a flowchart of a method for instructing a user to perform a combination of head and neck exercises.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details. Therefore, specific structural and functional details disclosed herein are not to be interpreted as limiting, but merely as a representative basis for teaching one skilled in the art to employ variously the present invention.

Referring to all Figures and embodiments described in this application, similarly-numbered parts (e.g., 118, 318, 418, etc.) may have similar descriptions, designs, and embodiments.

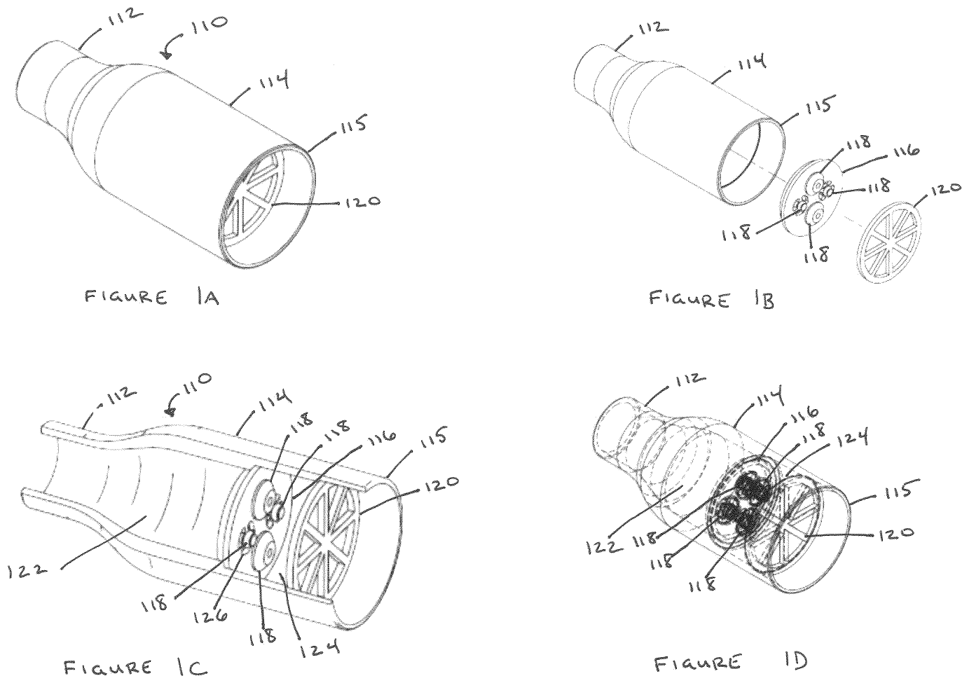

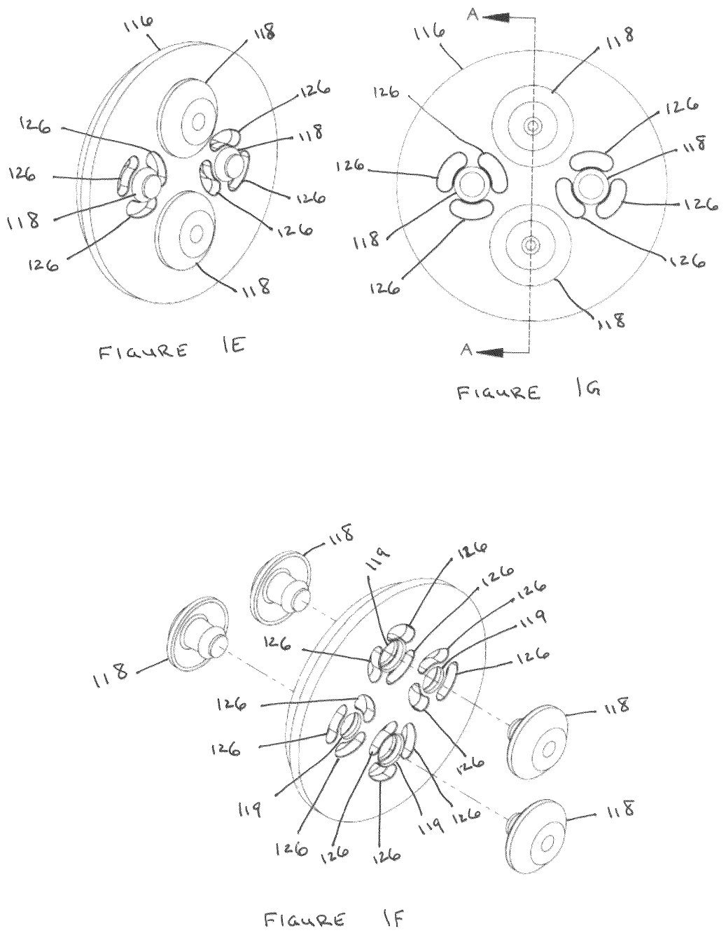

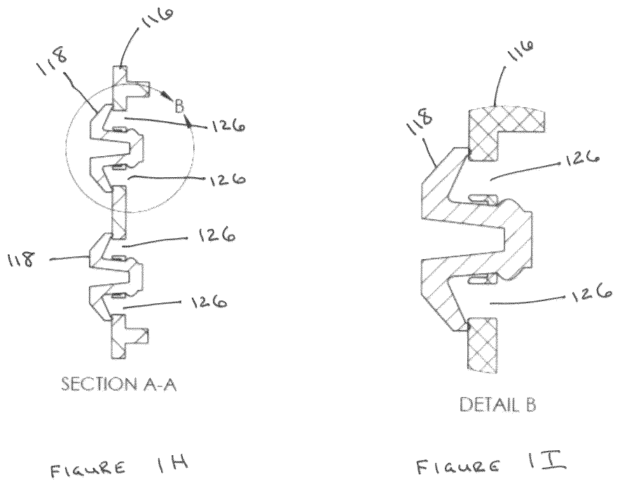

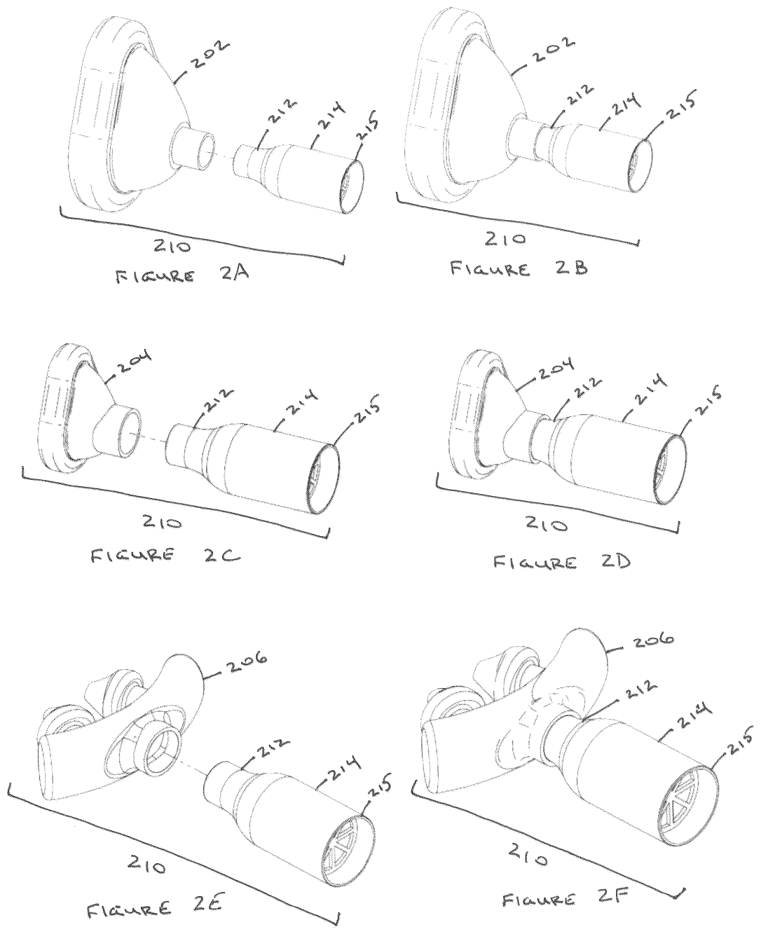

Referring to FIGS. 1A, 1B, 1C, 1D, 1E, 1F, 1G, 1H, and 1I, an embodiment of a head and neck exercise apparatus 110 may be used in the performance of isotonic and/or isometric head and neck exercises for sleep disordered breathing. These exercises can include exercises that require the generation of positive or negative pressure within the nose, oral cavity, and/or pharynx. These exercises may be breathing exercises, and, by extension, the exercise apparatuses or systems may be considered breathing apparatuses or breathing exercise systems, respectively. These exercises may improve sleep disordered breathing through a number of mechanisms, including changing muscle strength and/or endurance, muscle activity, neurologic (motor and/or sensory) function, coordination of muscle activation, tissue characteristics, and/or position of anatomic structures of the head and neck during wakefulness and/or sleep. Analogous to traditional isotonic and/or isometric exercises for muscles in other areas of the body, the use of head and neck exercise apparatuses or systems that provide resistance and/or feedback during training could provide a number of benefits, including facilitating exercise performance and monitoring.

The head and neck exercise apparatus 110 may include a member 114. In this embodiment, the member 114 is generally cylindrically-shaped with a proximal interface 112 having a similar general shape with a lesser diameter. The member 114 may define a passageway or proximal portion 122 there through. The proximal interface 112 may be positioned to a user's mouth or connected to another interface component that could permit a user to position the head and neck exercise apparatus 110 to a part of a user's head and neck such as the nose and/or the mouth. In this embodiment, the member 114 also defines a passageway or distal portion 124 there through. In other embodiments, the distal interface 115 may be positioned to a user's mouth or connected to another interface component that could permit a user to position the head and neck exercise apparatus 110 to a part of a user's head and neck such as the nose and/or the mouth. In other embodiments, the member 114, proximal interface 112, and other components may have different general shapes and sizes. In other embodiments, there may be zero or more than one proximal interface 112 and/or distal interface 115.

The member 114 may be considered a housing, within which there may be additional components of the head and neck exercise apparatus 110 or at least one component of at least one other head and neck exercise apparatus. A valve plate 116 may be operatively arranged with (e.g., disposed within, connected to, etc.) the member 114. The valve plate may cooperate with at least one valve mechanism 118 that is positioned within a valve stem hole 119; collectively, this may be defined as a valve plate assembly. In this embodiment, the valve plate 116 contains four valve stem holes 119, through which a valve mechanism 118 is positioned in each of them; of the four valve mechanisms 118, two are oriented in each direction. The valve plate 116 may separate the proximal portion 122 and distal portion 124 when all valve mechanisms 118 are in the closed position. A tamper guard 120 may be positioned to help shield the valve plate 116 and valve mechanisms 118 from physical contact.

In certain embodiments, the valve mechanisms 118 are biased towards the closed position unless at least one valve mechanism 118 is opened when the positive pressure in the proximal portion 122 is greater than a threshold pressure (e.g., 0.5 pounds per square inch), such threshold pressure to be determined by the properties of the member 114 (e.g., dimensions, etc.), valve plate 116 (e.g., thickness, etc.), and/or at least one valve mechanism 118 (e.g., stiffness, thickness, etc.). For example, if the pressure in the proximal portion 122 rises above the threshold pressure to open the at least one valve mechanism 118 but subsequently falls below the threshold pressure, the at least one valve mechanism 118 closes.

In this embodiment, because two valves are oriented in each direction, at least one valve mechanism 118 (but not more than two valve mechanisms 118) is opened when the positive pressure is greater than the threshold positive pressure of the respective valve mechanisms, such threshold pressure to be determined by the properties of the member 114 (e.g., dimensions, etc.), valve plate 116 (e.g., thickness, etc.), and/or valve mechanism 118 (e.g., stiffness, thickness, etc.). Similarly, in this embodiment at least one different valve mechanism 118 (but not more than two different valve mechanisms 118) is opened when the absolute value of the negative pressure (hereafter, “absolute negative pressure” or “negative pressure”) in the proximal portion 122 is greater than an absolute value of a threshold negative pressure (hereafter, “absolute threshold negative pressure” or “threshold negative pressure”, e.g., 0.5 pounds per square inch), such threshold negative pressure to be determined by the properties of the member 114 (e.g., dimensions, etc.), valve plate 116 (e.g., thickness, etc.), and/or valve mechanism 118 (e.g., stiffness, thickness, etc.). For example, if the negative pressure in the proximal portion 122 rises above the threshold negative pressure to open at least one valve mechanism 118 but subsequently falls below the threshold negative pressure, the at least one valve mechanism 118 closes.

In this application, unless specified otherwise, “pressure” hereafter refers to the magnitude of pressure, whether the absolute positive pressure or absolute negative pressure, and “threshold pressure” refers to the magnitude of pressure, whether threshold positive pressure or threshold negative pressure.

In this embodiment, each valve mechanism 118 individually has a threshold pressure that may be positive or negative pressure, but in other embodiments a valve mechanism may have a threshold positive pressure and a threshold negative pressure.

In this embodiment, the valve mechanisms 118 are substantially the same, but they could differ. The threshold pressure associated with positive pressure in the proximal portion 122 for opening the at least one valve mechanism 118 may differ from the threshold pressure associated with negative pressure in the proximal portion 122 for opening the at least one valve mechanism 118, even for the same head and neck exercise apparatus 110. All other embodiments described for positive pressure in the proximal portion could be configured for negative pressure.

When at least one valve mechanism 118 is open, the proximal portion 122 is fluidly connected to the distal portion 124, and airflow may occur between the proximal portion 122 and the distal portion 124 though at least one airflow channel 126. In this embodiment, the at least one valve mechanism 118 is presented as of the umbrella valve type; each at least one valve mechanism 118 forms a seal by extending beyond the associated airflow channels 126, thereby separating the proximal portion 122 from the distal portion 124 when the at least one valve mechanism 118 is closed. Airflow may occur, for example, from the proximal portion 122 to distal portion 124 with a head and neck exercise that requires generation of positive pressure in the proximal portion 122, if the positive pressure exceeds the threshold pressure, thereby opening the at least one valve mechanism 118 to permit airflow through at least one airflow channel 126 associated with the at least one valve mechanism 118. The threshold pressure of the at least one valve mechanism 118 may be associated with the construction and features of the valve mechanism 118 and/or properties of other components (valve plate 116, member 114, tamper guard 120, etc.) of the head and neck exercise apparatus 110. For a valve mechanism 118 of the umbrella valve type, the threshold pressure of the valve mechanism 118 would generally be greater with greater thickness of the valve plate 116. For this head and neck exercise using the head and neck exercise apparatus 110, this airflow could provide simple and immediate user feedback to indicate that the pressure (positive pressure or absolute negative pressure, with different directions of airflow for each) in the proximal portion 122 was greater than the relevant threshold pressure.

In other embodiments, the at least one valve mechanism 118 may open if the pressure in the proximal portion 122 rises above the threshold pressure but then remain open if the pressure in the proximal portion 122 subsequently falls below the threshold pressure. The valve mechanism may include some means to return the valve to the closed position (e.g., reset lever, etc.). In other embodiments, the valve mechanism 118 may be biased towards the open position unless it is closed when the pressure in the proximal portion 122 is greater than a threshold pressure, with all other potential configurations described elsewhere in this application.

Apparatuses for head and neck exercises for sleep disordered breathing ideally provide feedback regarding proper performance of the exercises. As opposed to traditional muscle strength and/or endurance training exercises where gross body movements enable monitoring of the exercise, a user does not easily visualize some head and neck muscles during movement or contraction. Feedback from an apparatus for head and neck exercises for sleep disordered breathing may indicate correct movements or muscle contractions through a number of means, including those that depend on generation of positive pressure or negative pressure within the nose, oral cavity, and/or pharynx of a sufficient magnitude (absolute pressure) or duration.

User feedback could be based on airflow through an apparatus, any sensory signal (visual, auditory, or tactile), and/or objective measurement that may provide data in real-time or in a delayed fashion (e.g. with recording or transmission). The simplicity of the valve mechanism 118 avoids the need for a pressure transducer or other means that may be complex or expensive, as in many medical apparatuses that may emphasize precision over simplicity and low cost. Simplicity and low cost are more important for head and neck exercises for sleep disordered breathing than in most medical applications. For example, a user may perform these exercises in a wide range of settings over a period of time, where inexpensive and easily portable apparatuses would be favored. In other embodiments, the head and neck exercise apparatus 110 may include a mechanism for substitution of at least one of the valve plate 116 and/or at least one valve mechanism 118; substitution could provide additional benefits, including decreased cost. Sensory signals are well-known in the art and could be incorporated into the head and neck exercise apparatus 110. Objective measurements may relate to the act of performance of the exercise (e.g., adherence to an exercise regimen, etc.) or the nature of the performance (e.g., pressure transducer, etc.). Objective measurements may be recorded in the head and neck exercise apparatus or transmitted (wired, wireless, etc.) for evaluation in real-time or a delayed fashion, with or without storage of the data.

A head and neck exercise system may include at least one head and neck exercise apparatus with at least one threshold pressure or airflow resistance that is adjustable or at least two head and neck exercise apparatuses, each with at least one threshold pressure that may be adjustable or not adjustable. The threshold pressure or airflow resistance may be adjustable or variable during the performance of the head and neck exercise. The head and neck exercise system could be designed to provide a range of threshold pressures for the performance of head and neck exercises. Selection of a threshold pressure for performance of a head and neck exercise may be based on one of more factors, including an ability of the user to perform the head and neck exercise or a different head and neck exercise, using the head and neck exercise apparatus 110 or another head and neck exercise apparatus, with the threshold pressure or another threshold pressure, for a number of repetitions or duration of time or with a muscle strength or a muscle tension required to perform the head and neck exercise using the head and neck exercise apparatus, the another head and neck exercise apparatus, or a different head and neck exercise apparatus. Assessment for selecting a threshold pressure may involve one or more types of head and neck exercise apparatuses, and an apparatus used specifically for testing muscle strength or tension may be considered one type of head and neck exercise apparatus.

A number of respiratory apparatuses used in medical environments are capable of monitoring positive pressure or negative pressure, but these may not be well-suited to the performance of these head and neck exercises for sleep disordered breathing. Respiratory apparatuses are often developed for disorders affecting the lungs rather than the nose, oral cavity, and/or pharynx. This leads to features, such as freely permitting airflow through the apparatus (in order to monitor airflow without disturbance), that may be unfavorable for some head and neck exercises for sleep disordered breathing. These apparatuses or their feedback mechanisms may be expensive or complex, require electrical parts, or be designed to offer precision in measurements of pressure or airflow. These features may not be necessary with exercises for sleep disordered breathing. These and other limitations may have prevented the use of respiratory apparatuses in head and neck exercises for sleep disordered breathing and treatment of many other conditions.

Exercise apparatuses used in other areas of the body (for example, including weights) have been adapted for use in the head and neck region, but their uses have generally been limited to strengthening muscles for chewing or teeth clenching that may not be suited to exercises for sleep disordered breathing. The unique nature of head and neck exercises for sleep disordered breathing requires different apparatus designs.

Three types of apparatuses have been proposed for head and neck exercises for sleep disordered breathing: (1) brushes or apparatuses with a roughly linear, solid design that can stroke or brush the tongue; (2) tubes designed for drawing liquid from a container connected to a pressure transducer; and (3) positive end-expiratory pressure relief valves utilizing a coil spring mechanism. There are a number of limitations of these apparatuses. The first type is not designed for resistance training exercises and does not provide meaningful feedback regarding many of these exercises. The second is unnecessarily complex and expensive, which may not be favorable for many reasons, including those outlined above.

Positive end expiratory pressure relief valves placed to the mouth have been proposed for use during exercise or for expiratory muscle training, primarily for disorders affecting the lungs such as chronic obstructive pulmonary disease. These apparatuses provide resistance training for the chest wall muscles relevant to these pulmonary disorders, and the threshold pressure level may be determined by a coil spring mechanism that enables adjustment over a wide (and continuous) range. These apparatuses have a number of limitations for the treatment of sleep disordered breathing. First, they have been used largely in expiration only. In addition, placement to the mouth may train muscles associated with oral breathing, but placement to the nose may train muscles associated with nasal breathing that may be generally more favorable in patients with sleep disordered breathing.

These devices may be well-suited to chronic obstructive pulmonary disease in which an exercise program generally consists of a single exercise, but exercises for sleep disordered breathing typically involve more than one exercise. With multiple exercises, complexity (e.g., a single apparatus with a threshold pressure that must be adjusted) may not be favorable. In contrast, a selected range of similar apparatuses with different threshold pressures may be more straightforward and may enable the construction of simpler apparatuses. There are other limitations and drawbacks of apparatuses that are available currently.

The valve mechanism 118 may have any design that performs the functions as specified. In other embodiments, the valve mechanism 118 may contain at least two components. In other embodiments, the valve mechanism 118 may have a means of adjustment (application of pressure to a portion, placement of a foreign material in the valve stem, etc.) to alter its properties (e.g., dimensions, resistance to opening, etc.). Adjustment of the threshold pressure of the valve mechanism 118 may enable a head and neck exercise apparatus 110 to encompass a wider range of threshold pressures and reduce the number of apparatuses that would be incorporated into an exercise program. However, there also may be greater complexity, including challenges in ensuring that the adjustment provides precise and/or accurate performance characteristics (threshold pressure, etc.).

In other embodiments, the valve mechanism 118 may be configured to respond both to positive and to negative pressure. In other embodiments, the head and neck exercise apparatus 110 may be configured to allow substitution of the valve mechanism 118. In other embodiments, the member 114 may have no distal portion 124, enabling the proximal portion 122 to communicate directly to the external environment when the valve mechanism 118 is open.

In this embodiment, the valve mechanisms 118 are distinct from the valve plate 116, but in other embodiments any component of at least one valve mechanism 118 may be manufactured in continuity with any part of the valve plate 116 or head and neck apparatus 110.

A single physical device or member can function as at least two head and neck exercise apparatuses 110, whether for a single head and neck exercise or for at least two head and neck exercises. In addition to the differences that exist for at least two head and neck exercises, there are multiple means by which this may occur: adjustment and/or substitution of at least one valve mechanism 118, different orientations of at least one valve mechanism 118, positioning the proximal interface 112 and/or distal interface 115 to the nose and/or mouth (without and/or with the use of a mask interface), etc. Adjustment or substitution of a valve mechanism 118 or other component may modify the threshold pressure and be considered a modification, just as the ability of a valve mechanism 118 to be adjusted or substituted may be considered modifiable. A single physical device functioning as at least two head and neck exercise apparatuses may provide lesser cost, lesser complexity, or other benefits.

A method for performing breathing exercises with a breathing exercise system including at least one breathing apparatus may comprise positioning a member defining an open proximal portion to a nose of a user such that breathing by the user through the open proximal portion results in anterior movement of a soft palate of the user to open space in an area of a throat of the user behind the soft palate to create a continuous air pathway from the nose to lungs of the user; performing by the user a first breathing exercise via the continuous air pathway in which the user generates, for a predetermined number of repetitions or duration of time, a pressure within the open portion of the apparatus that exceeds a threshold pressure of a valve mechanism biased towards a closed position to cause the valve mechanism to open such that the open proximal portion is fluidly connected to a distal portion and the lungs to train muscles of the user; positioning the open proximal portion of the member to a nose of a user such that breathing by the user through the open proximal portion results in anterior movement of a soft palate of the user to open space in an area of a throat of the user behind the soft palate to create a continuous air pathway from the nose to lungs of the user; and performing by the user the second breathing exercise via the continuous air pathway in which the user generates, for a predetermined number of repetitions or duration of time, a pressure within the open proximal portion that exceeds a threshold pressure of a valve mechanism biased towards a closed position to cause the valve mechanism to open such that the open proximal portion is fluidly connected to the distal portion and the lungs to train muscles of the user.

The proximal interface 112 may be placed between the lips such that it extends inside the oral cavity to assist with maintaining the apparatus position or achieving an airtight communication with the oral cavity. The proximal interface 112 may be of any configuration or placement (to the nose, to the lips, between the lips, inside the cheeks, between the teeth, above the tongue, etc.). The proximal interface 112 may be configured to favor an orientation of at least one structure of the face or oral cavity. A mask interface, mouthpiece, or other component may cooperate with the proximal interface 112 to facilitate the placement against a portion of the head and neck of the user. Such an orientation could be desirable in the performance of head and neck exercises for sleep disordered breathing.

In this embodiment, the proximal interface 112 is manufactured in continuity with the member 114. In other embodiments, the proximal interface 112 may be separable from the member 114. In these embodiments, the proximal interface 112 and member 114 may have a coupling element, with a possible mechanism to enhance their cooperation, such as a locking or snap mechanism. In other embodiments, any suitable proximal interface 112 or design of the member 114 may be used as an interface between the user and apparatus.

The head and neck exercise apparatus 110 may be configured with a mechanism to sense pressure in the proximal portion 122 and/or distal portion 124. The head and neck exercise apparatus 110 may be configured with a means for transmission of real-time or stored data (e.g., exercise performance, pressure, etc.).

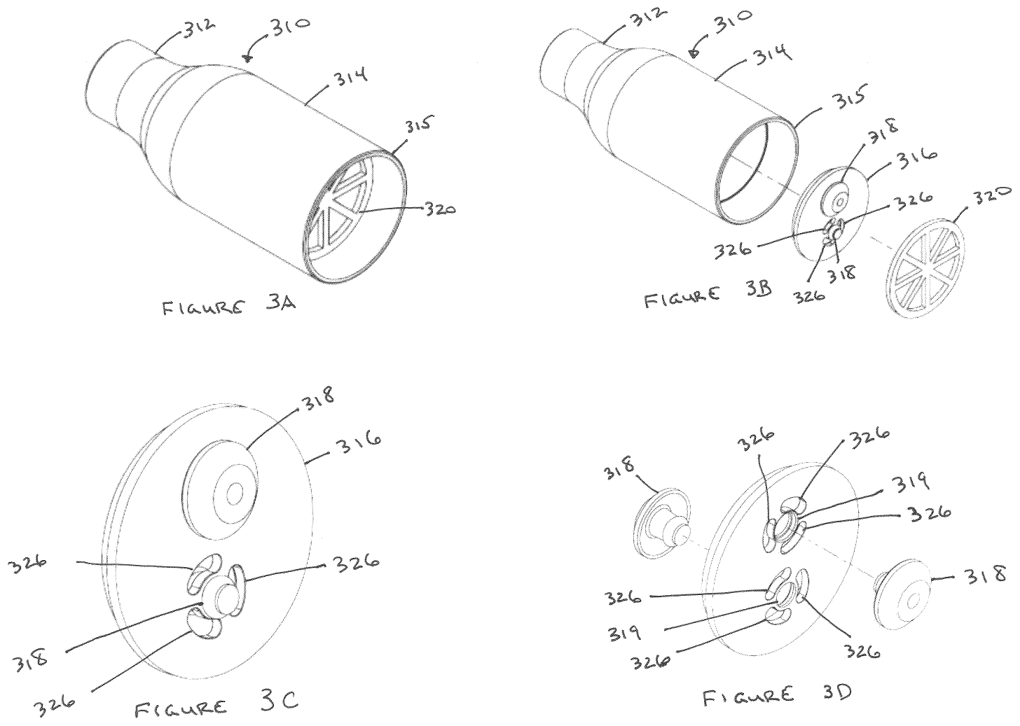

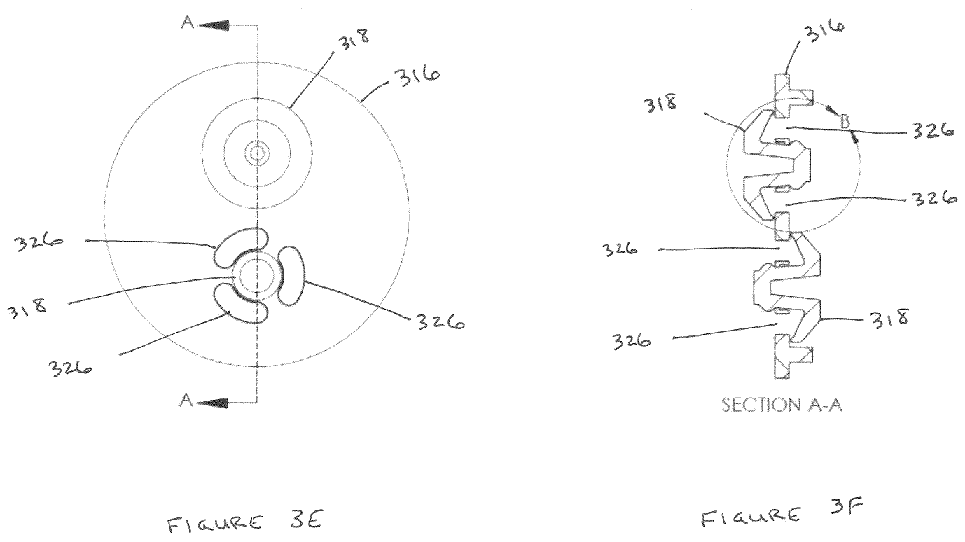

Referring to FIGS. 2A, 2B, 2C, 2D, 2E, and 2F, other embodiments of a head and neck exercise apparatus 210 are shown. Each head and neck exercise apparatus 210 may include a mask interface that cooperates with it at the proximal interface 212. The mask interface may be of various types, including an oronasal mask interface 202, nasal mask interface 204, or nasal pillows mask interface 206. Other mask interfaces are possible, including those that extend into the nose and/or the oral cavity. These mask interface types have been used in various applications, including with positive airway pressure therapy, where they may be positioned against a portion of a user's head and neck to enable a seal between the mask interface and the user. An oronasal mask interface 202 is designed to enable a seal around a user's nose and mouth; this facilitates placement of the head and neck exercise apparatus 210 to a user's nose and mouth. A nasal mask interface 204 is designed to enable a seal around a user's nose; this facilitates placement of the head and neck exercise apparatus 210 to a user's nose. A nasal pillows mask interface 206 is designed to enable a seal with a user's nostrils; this facilitates placement of the head and neck exercise apparatus 210 to a user's nose. The head and neck exercise apparatus 210 may be configured to include more than one type of mask interface.

One goal of treating sleep disordered breathing is maintaining a patent upper airway, enabling a continuous air pathway from the nose to the lungs. Breathing through the nose may result in anterior movement of the soft palate to open the space for breathing in the area of the throat behind the soft palate, whereas breathing through the mouth may result in posterior movement of the soft palate to open the space for breathing between the tongue and soft palate. This may be particularly true for performance of head and neck exercises involving active breathing through the nose vs. mouth. Breathing through the nose may also involve closure of the space between the tongue and soft palate. Breathing through the mouth may also narrow the space for breathing in the throat directly or indirectly, as compared to breathing through the nose; mechanisms may include posterior displacement of the tongue, decrease in stretch and/or tension of the soft tissues surrounding the throat, loss of nasal airflow that may provide sensory input to neuromuscular reflexes important in maintaining a patent upper airway, and/or other sources of increased upper airway collapsibility.

For purposes of this application, positioning a head and neck exercise apparatus “to the user's nose” encompasses positioning to the nose (for breathing through the nose alone) or positioning to the nose and mouth (for breathing through the nose and mouth).

The mask interface may reversibly or irreversibly cooperate with the member 214 at the proximal interface 212. A coupling mechanism, such as a locking or snap mechanism, may facilitate this cooperation. A reversible cooperation may allow the member 214 to cooperate with different mask interfaces. A reversible cooperation may also allow the mask interface to cooperate with the member 214 of different head and neck exercise apparatuses 210 that may have different properties. Cooperation with the member 214 of different head and neck exercise apparatuses 210 could enable an user to perform head and neck exercises for sleep disordered breathing using apparatuses with a range of threshold pressures that could be desirable. An exercise program may incorporate exercises that require different threshold pressures, and the use of head and neck exercise apparatuses 210 with a range of threshold pressures or multiple apparatuses would reduce the need for adjustment or calibration of any single head and neck exercise apparatus 210. Adjustment and calibration can introduce cost, complexity, and expenditure of time and effort, any of which may be undesirable.

In this embodiment, the mask interfaces are distinct from the head and neck exercise apparatus, but in other embodiments any component of the mask interface may be manufactured in continuity with any part of the head and neck exercise apparatus 210. In this embodiment, the mask interfaces cooperate with the member at the proximal interface 212. In other embodiments, a mask interface may connect directly to the member 214 without a proximal interface 212, or the mask interface may cooperate with the head and neck exercise apparatus 210 at the distal interface 215.

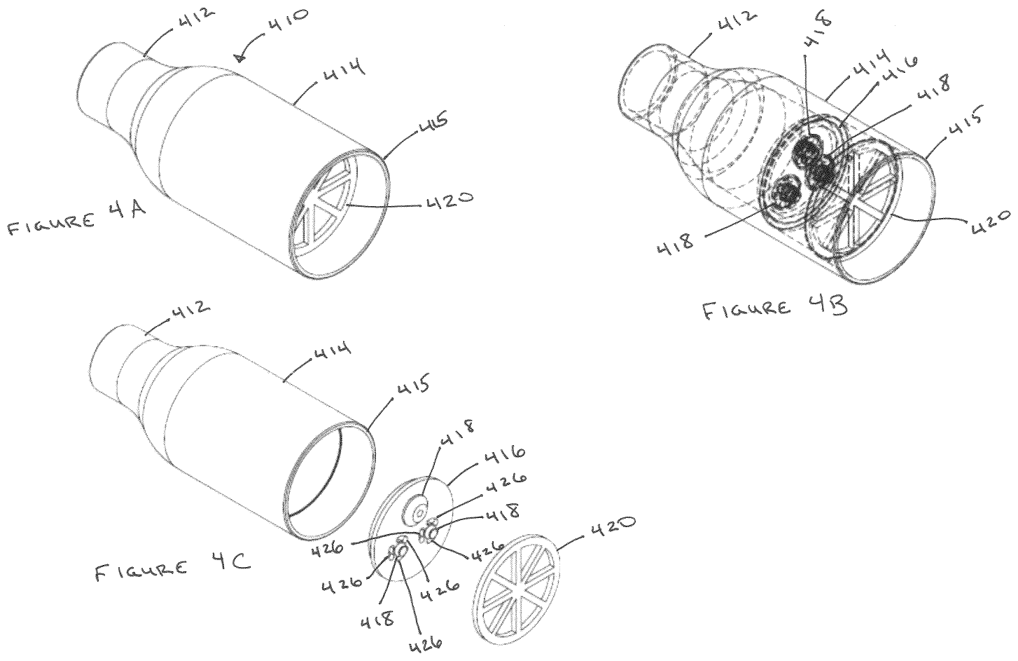

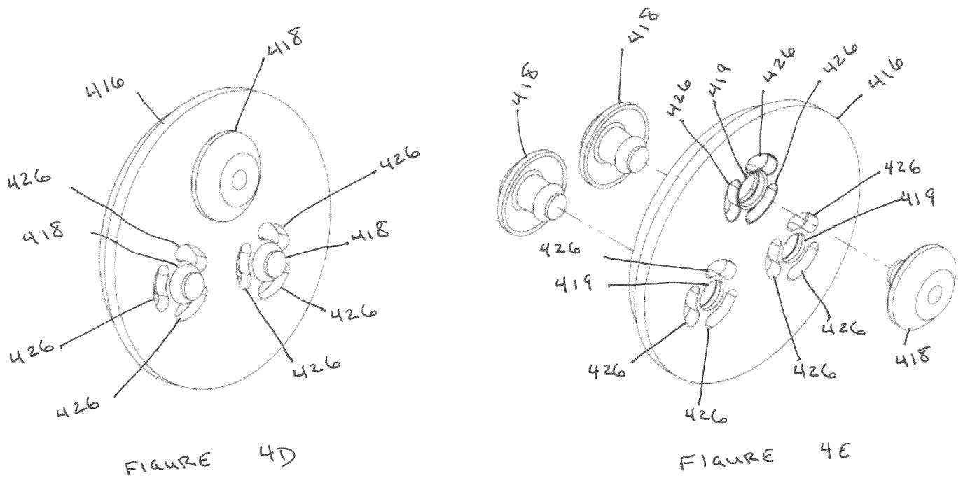

Referring to FIGS. 3A, 3B, 3C, 3D, 3E, and 3F, another embodiment of a head and neck exercise apparatus 310 is shown. In this embodiment, the valve plate 316 contains two valve stem holes 319, through which a valve mechanism 318 is positioned in each one; of the two valve mechanisms 318, one is oriented in each direction.

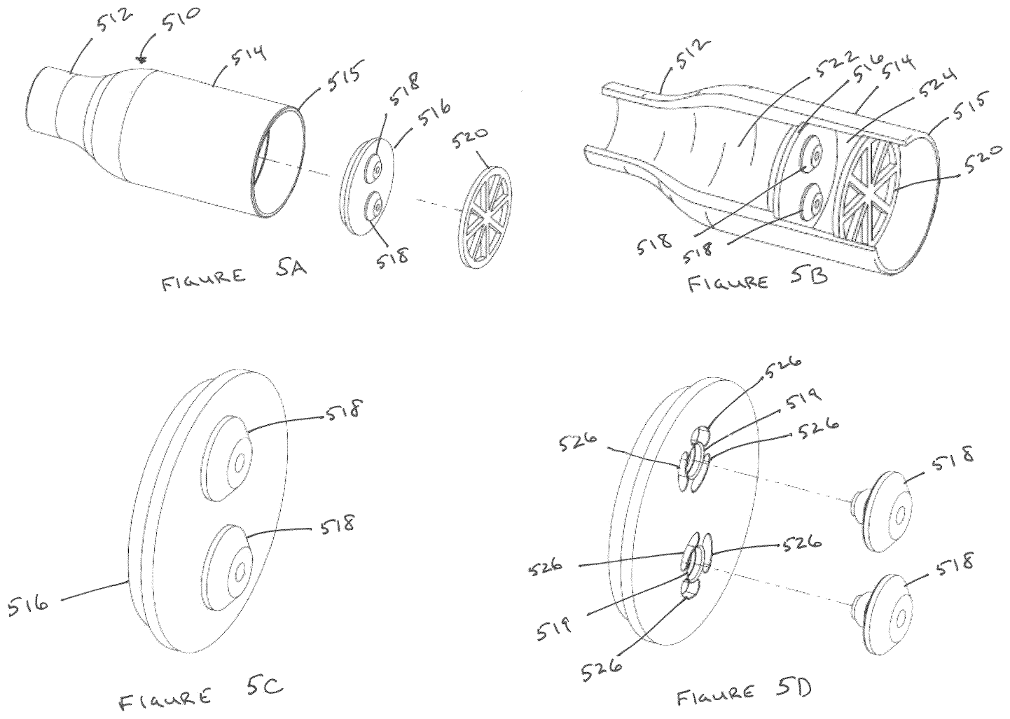

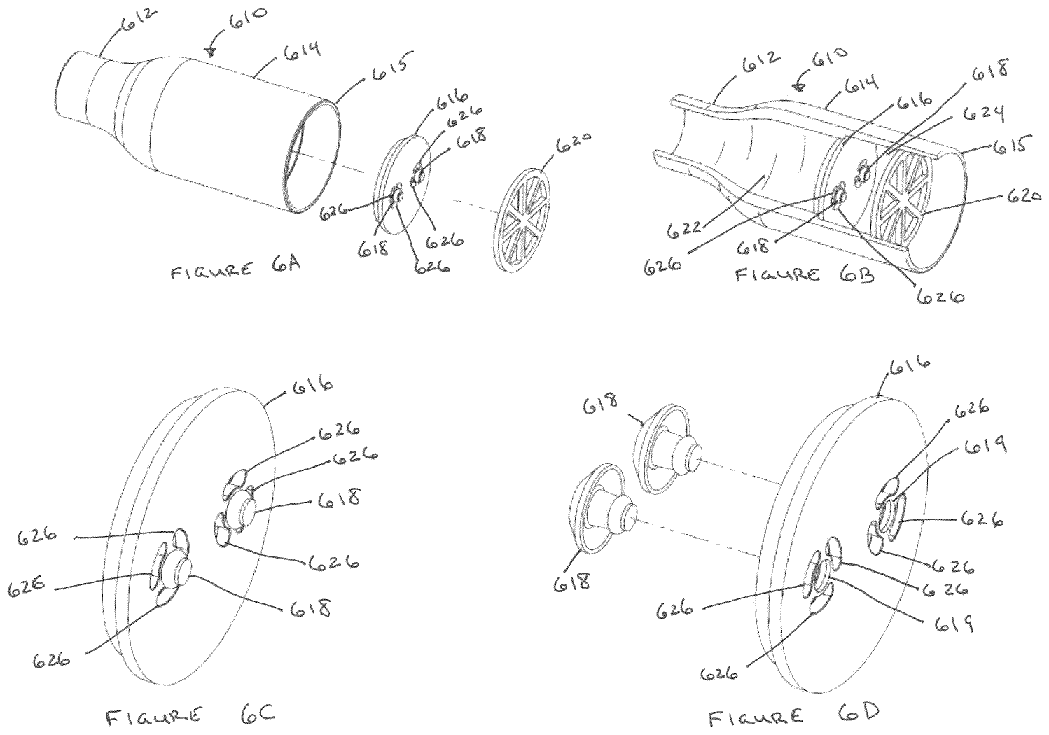

Referring to FIGS. 4A, 4B, 4C, 4D, and 4E, another embodiment of a head and neck exercise apparatus 410 is shown. In this embodiment, the valve plate assembly includes a valve plate 416 with three valve stem holes 419, through which a valve mechanism 418 is positioned in each one; of the three valve mechanisms 418, one is oriented in one direction, and two are oriented in the opposite direction. In other embodiments, the valve plate may include a single valve mechanism 418 or at least two valve mechanisms 418 oriented in a single direction.

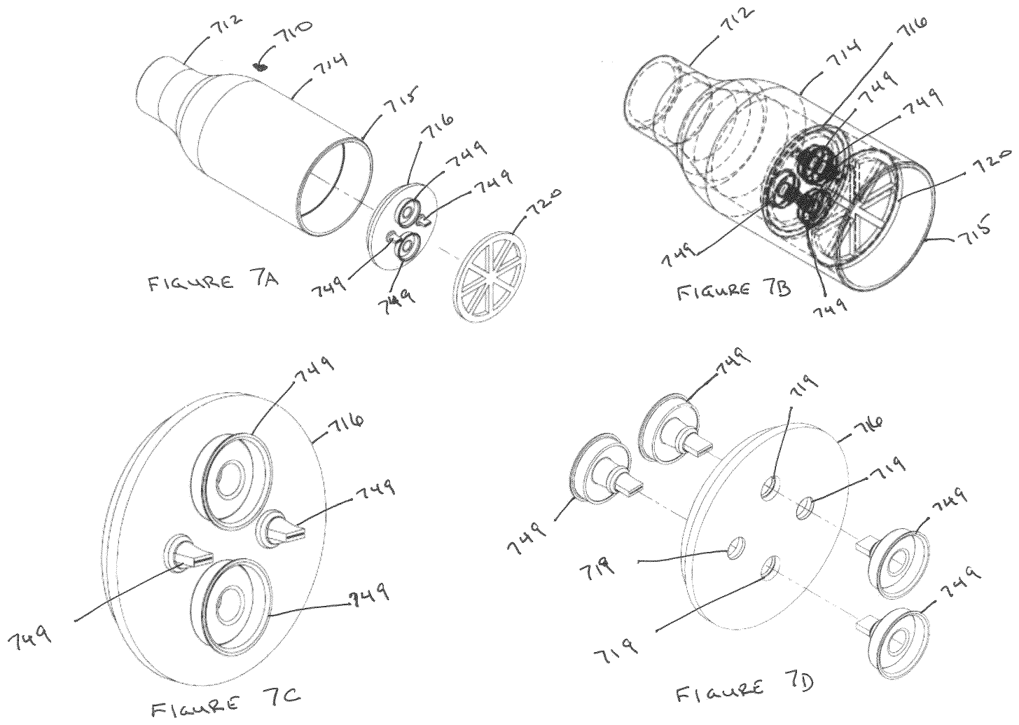

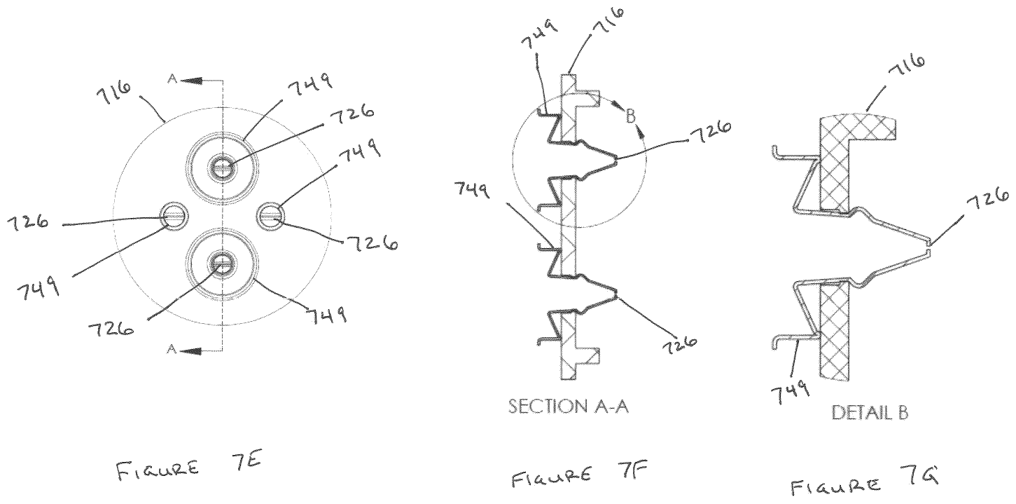

Referring to FIGS. 5A, 5B, 5C, and 5D, another embodiment of a head and neck exercise apparatus 510 is shown. In this embodiment, the valve plate assembly includes a valve plate 516 with two valve stem holes 519, through which a valve mechanism 518 is positioned in each one; in this embodiment, the two valve mechanisms 518 are oriented in the same direction. In this embodiment, the head and neck exercise apparatus 510 may be configured to open the at least one valve mechanism 518 only with positive pressure in the proximal portion 522 that exceeds the threshold pressure. In other embodiments, the valve plate may include a single valve mechanism 518.

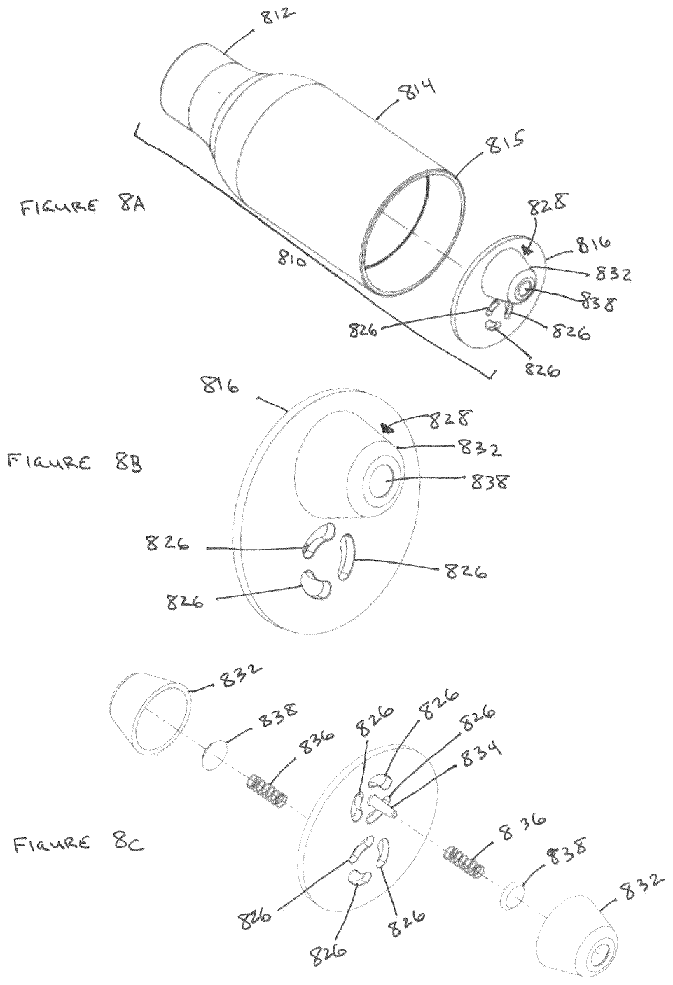

Referring to FIGS. 6A, 6B, 6C, and 6D, another embodiment of a head and neck exercise apparatus 610 is shown. In this embodiment, the valve plate assembly includes a valve plate 616 with two valve stem holes 619, through which a valve mechanism 618 is positioned in each one; in this embodiment, the two valve mechanisms 618 are oriented in the same direction. In this embodiment, the head and neck exercise apparatus may be configured to open the valves only with negative pressure in the proximal portion 622 that exceeds the threshold pressure. In other embodiments, the valve plate may include a single valve mechanism 618.

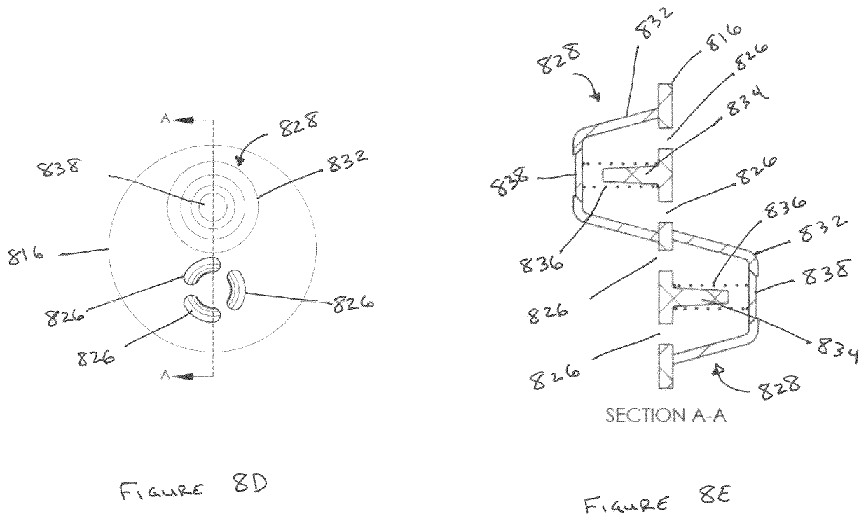

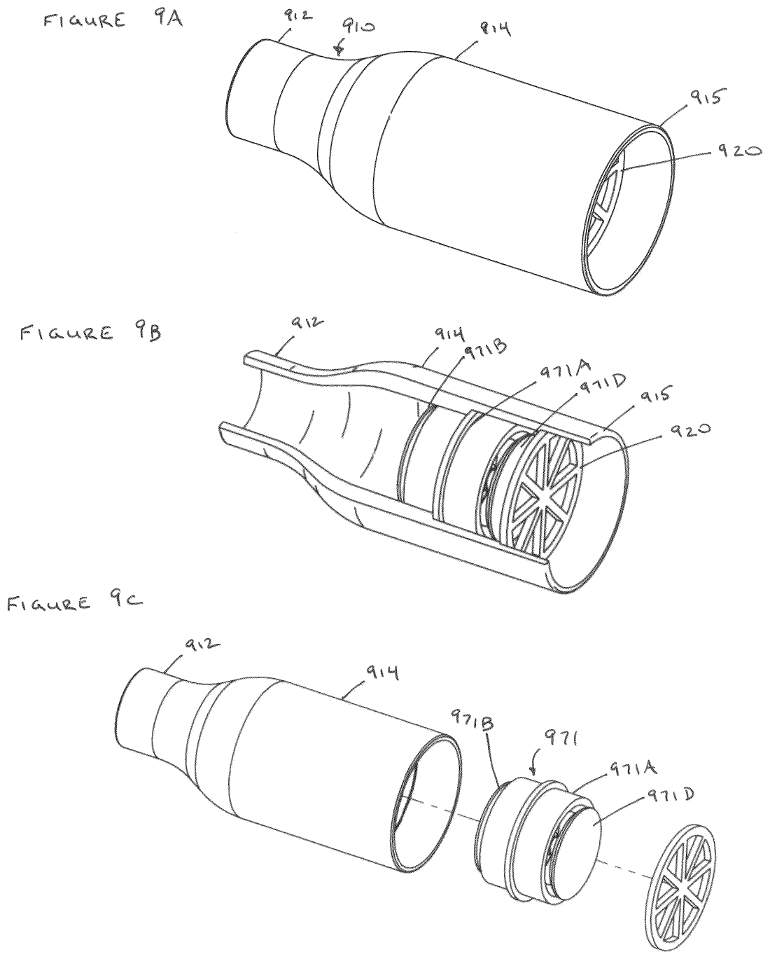

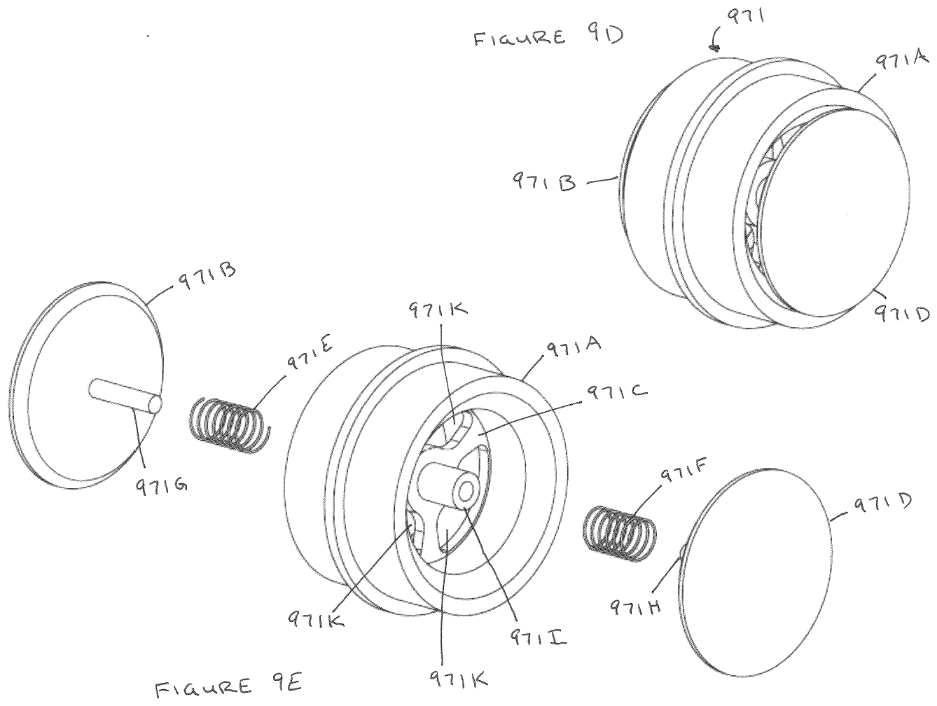

The valve mechanisms 118 (FIG. 1), 318 (FIG. 3), 418 (FIG. 4), 518 (FIG. 5), and 618 (FIG. 6) are presented as of the umbrella valve type, but other valve mechanism types such as duckbill valve, spring plunger valve, deflection valve, reed valve, leaf valve, ball valve, check valve, gate valve, plug valve, diaphragm valve, and solenoid valve are possible. These valve mechanism types would be associated with corresponding changes in the configuration of features associated with the valve mechanism, and multiple valve types may be used within the same apparatus, housing, or system. Referring to FIGS. 7A, 7B, 7C, 7D, 7E, 7F, and 7G, another embodiment of a head and neck exercise apparatus 710 is shown. The valve plate assembly includes a valve plate 716 and four duckbill valve mechanisms 749 that are each positioned within a valve stem hole 719; two duckbill valve mechanisms 749 are oriented in each direction. When any duckbill valve mechanism 749 is open, airflow may occur through the airflow channel 726 in the central portion of the duckbill valve mechanism 749. In this embodiment, the airflow channel 726 is contained within the duckbill valve mechanism 749, and there are no separate airflow channels.

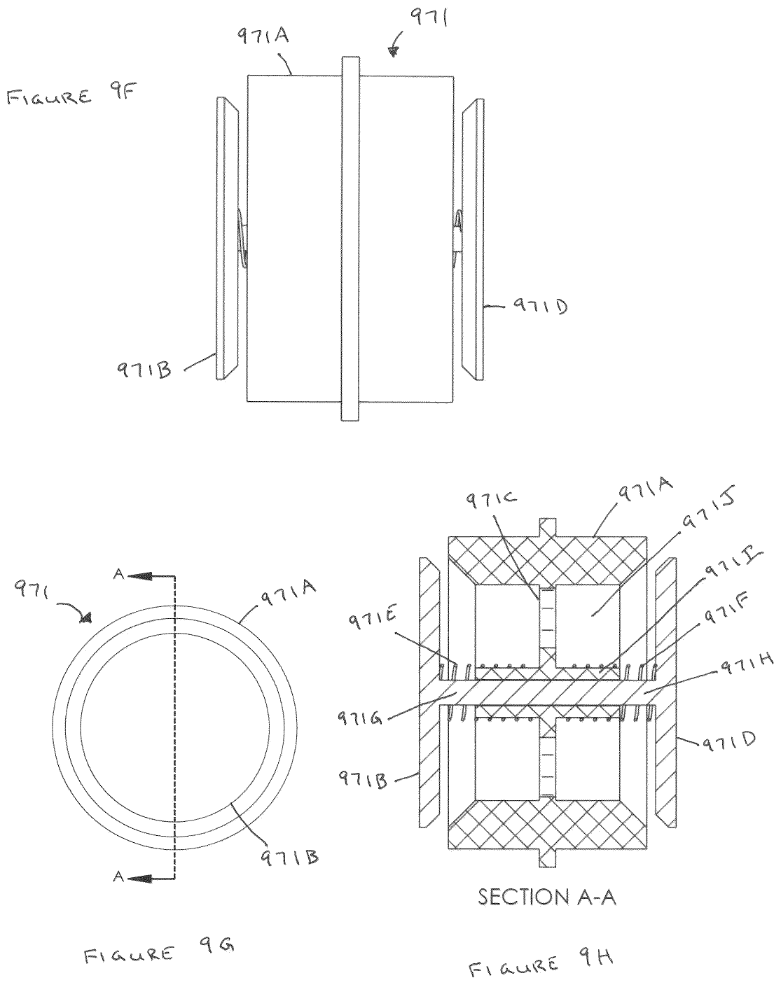

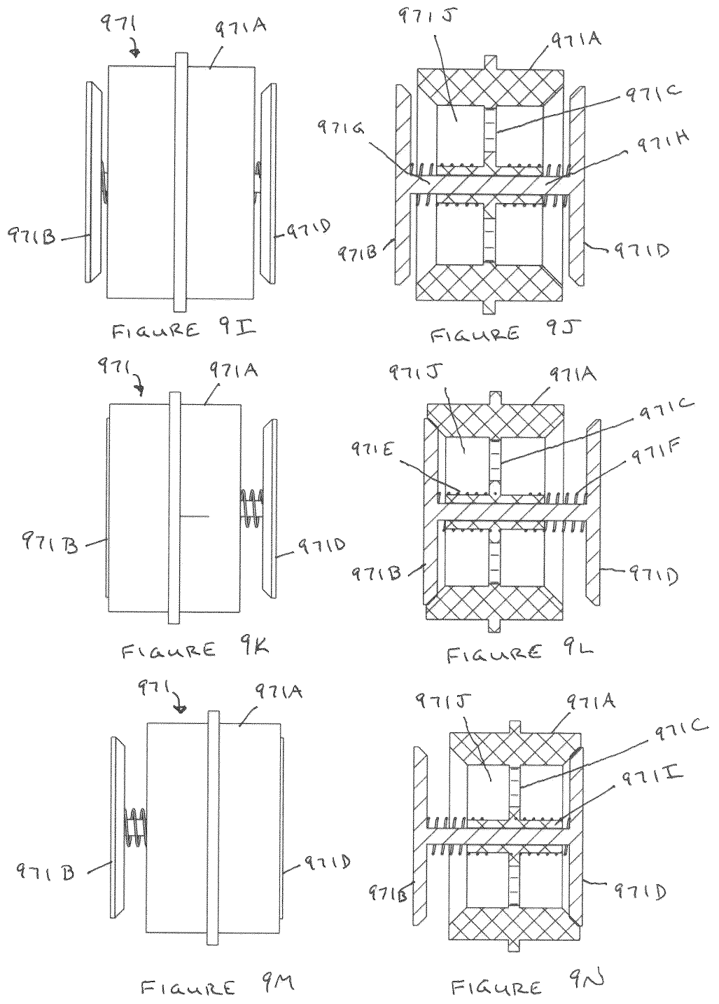

Referring to FIGS. 8A, 8B, 8C, 8D, and 8E, another embodiment of a head and neck exercise apparatus 810 is shown. In this embodiment, the valve mechanisms are of the spring plunger type. The valve plate assembly includes a valve plate 816 and two spring valve mechanisms 828, with one oriented in each direction. Within each spring valve mechanism 828, a spring 836 is positioned over a post 834 so that the spring 836 applies pressure against a cap 838 to form a seal against the valve case 832. The post 834 is secured to the valve plate 816. When the positive pressure in the proximal portion of the member 814 is greater than a threshold pressure, the positive pressure may displace the cap 838 of one spring valve mechanism 828 towards the distal end of the head and neck apparatus 810 and disrupt the seal against the valve case 832. This disruption may allow airflow through the separation between the cap 838 and valve case 832 and subsequently through the airflow channels 826. Similarly, when the negative pressure in the proximal portion of the member 814 is greater than a threshold pressure, the negative pressure may displace the cap 838 of the other spring valve mechanism 828 towards the proximal end of the head and neck exercise apparatus 810 and disrupt the seal against the valve case 832. This disruption may allow airflow through the separation between the cap 838 and valve case 832 and subsequently through the airflow channels 826 in the opposite direction. When at least one spring valve mechanism 828 is open, the proximal portion of the member 814 is fluidly connected to the distal portion of the member 814.

The present embodiment includes two spring valve mechanisms 818 that open specifically to positive or negative pressure, but in other embodiments at least one spring valve mechanism 818 with a different configuration could open in response to either positive or negative pressure in the proximal portion of the member 814.

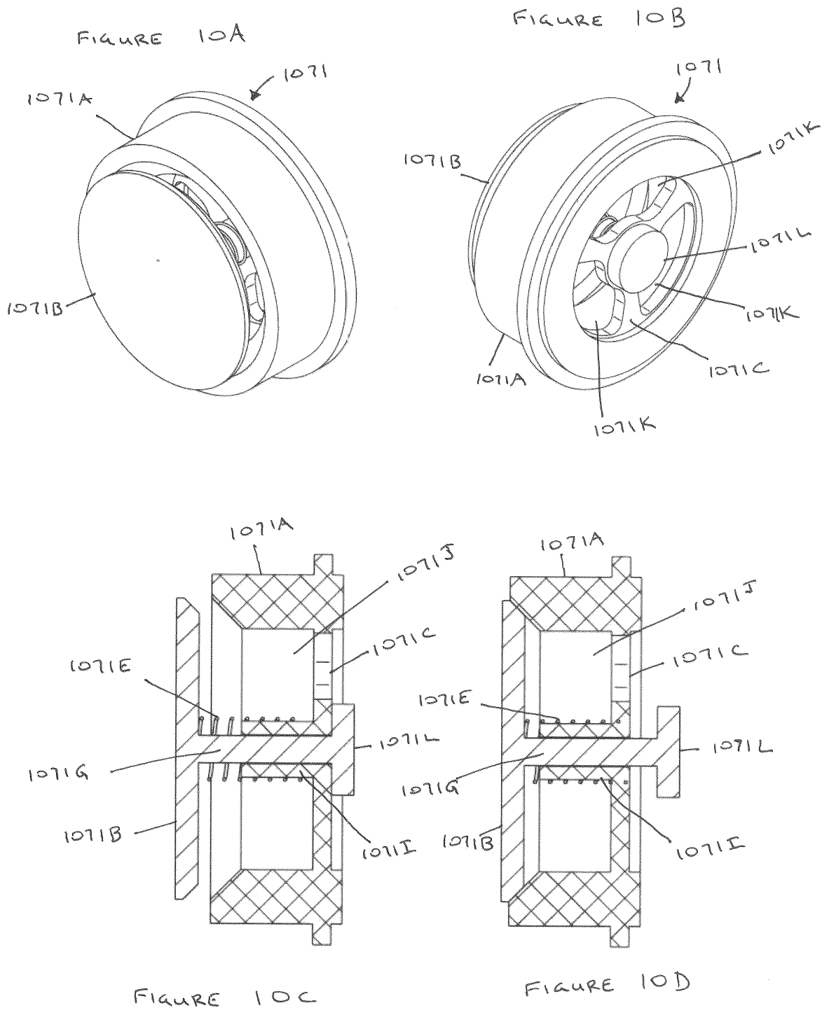

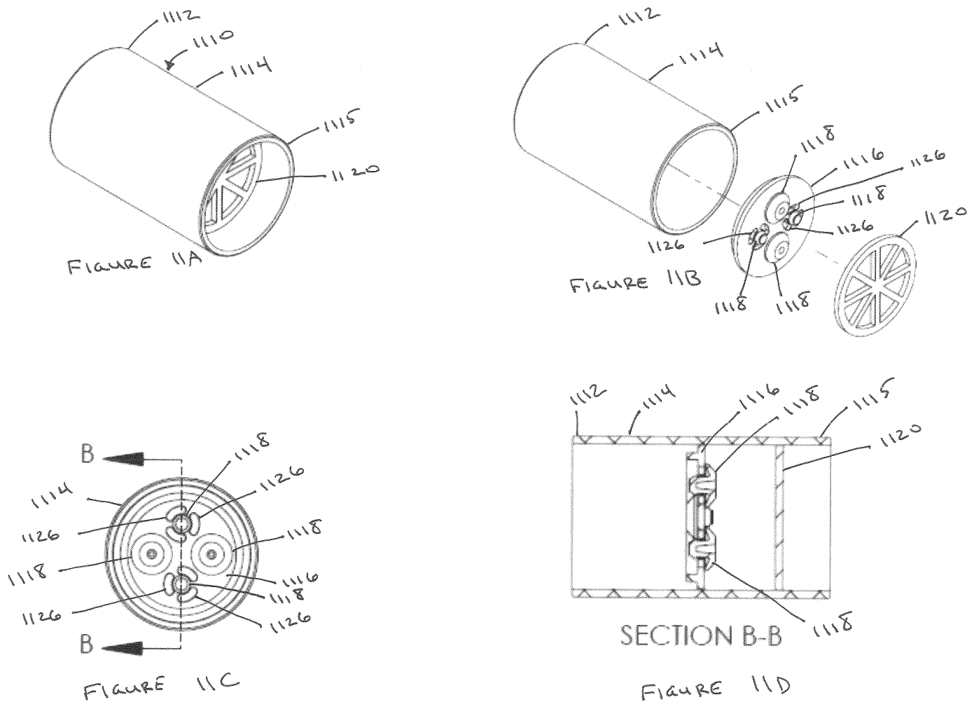

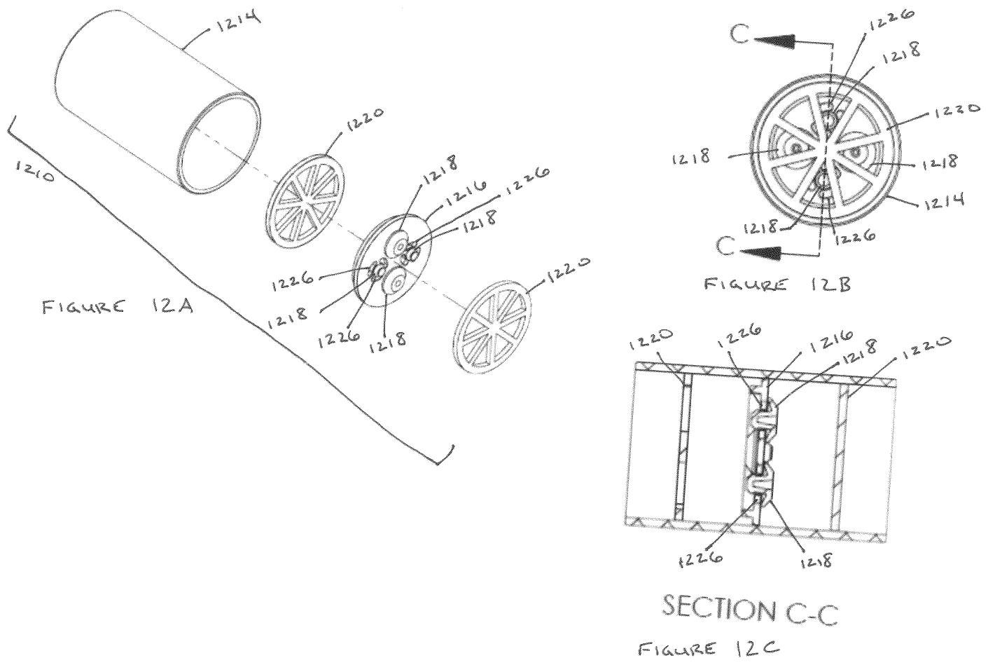

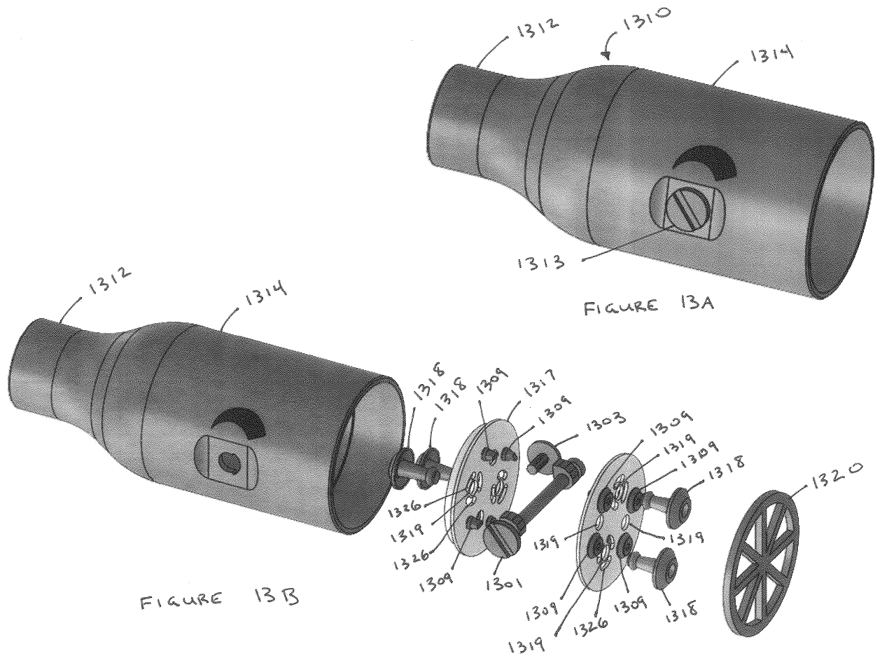

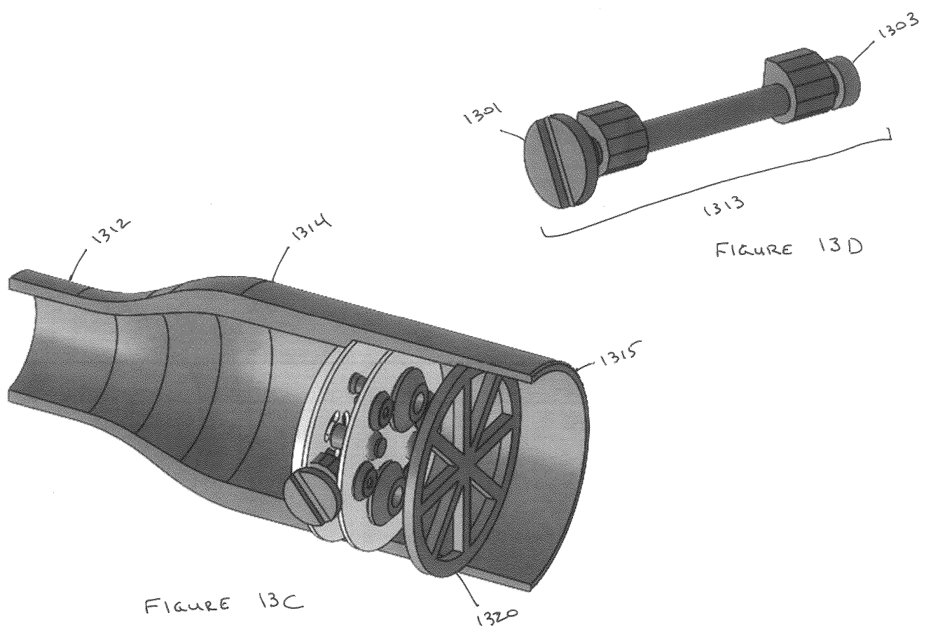

Referring to FIGS. 9A, 9B, 9C, 9D, 9E, 9F, 9G, 9H, 9I, 9J, 9K, 9L, 9M, and 9N, another embodiment of a head and neck exercise apparatus 910 is shown. The valve assembly 971 may be considered a valve mechanism. The valve assembly 971 may be operatively arranged with the member 914, such that airflow may only occur through the member 914 by passing through the valve assembly 971. The valve assembly 971 may include a valve assembly housing 971A, within which there may be a proximal valve assembly plate 971B, middle valve assembly plate 971C, distal valve assembly plate 971D, proximal valve assembly spring 971E, distal valve assembly spring 971F, proximal valve assembly plate connector 971G, distal valve assembly plate connector 971H, and middle valve assembly plate interface 9711. The valve assembly housing 971A may define a passageway or central portion 971J. The middle valve assembly plate 971B may define at least one middle valve plate airflow channel 971K. The proximal valve assembly plate 971B and distal valve assembly plate 971D may be linked via a connection between the proximal valve assembly plate connector 971G and distal valve assembly plate connector 971H; the connection may occur through the middle valve assembly plate 971C via the middle valve assembly plate interface 9711. The connection may be reversible or irreversible.

The proximal valve assembly spring 971E and distal valve assembly spring 971F may maintain the proximal valve assembly plate 971B and distal valve assembly plate 971D, respectively, in positions at rest that bias the valve assembly 971 towards an open position. At rest, the proximal valve assembly spring 971E and distal valve assembly spring 971F may contact the middle valve assembly plate 971C that is in a stable position within the valve assembly 971. In this embodiment, the proximal valve assembly spring 971E and distal valve assembly spring 971F are presented as of the compression spring type, but other spring types (e.g., extension, torsion, disk, etc.) are possible. Multiple spring types may be used in any combination within the same apparatus, housing, or system.

In this embodiment, the valve assembly 971 may be biased towards the open position unless it is closed when the positive pressure in a proximal portion 922 is greater than a threshold pressure, with all other potential configurations described elsewhere in this application. When the positive pressure in the proximal portion 922 is greater than a threshold pressure, the proximal valve assembly plate 971B may be displaced towards the middle valve assembly plate 971C, and the proximal valve assembly plate 971B may form a seal against the valve assembly housing 971A to close the valve assembly 971 (FIGS. 9K and 9L). The proximal portion 922 may therefore be separated from the central portion 971J.

Similarly, in this embodiment the valve assembly 971 may be biased towards the open position unless it is closed when the negative pressure in the proximal portion 922 is greater than another threshold pressure. When the negative pressure in the proximal portion 922 is greater than the another threshold pressure, the distal valve assembly plate 971D may be displaced towards the middle valve assembly plate 971C, and the distal valve assembly plate 971D may form a seal against the valve assembly housing 971A to close the valve assembly 971 (FIGS. 9M and 9N). The central portion 971J may therefore be separated from a distal portion 924.

The threshold pressures of the valve assembly 971 may be associated with the construction and features of the valve assembly 971, the components of the valve assembly 971, and/or properties of other components (member 914, tamper guard 920, etc.) of the head and neck exercise apparatus 910.

In this embodiment, the valve assembly is presented as of the plug valve type, but other valve types (shut-off valve, valve with spring elements, flap valve, etc.) are possible.

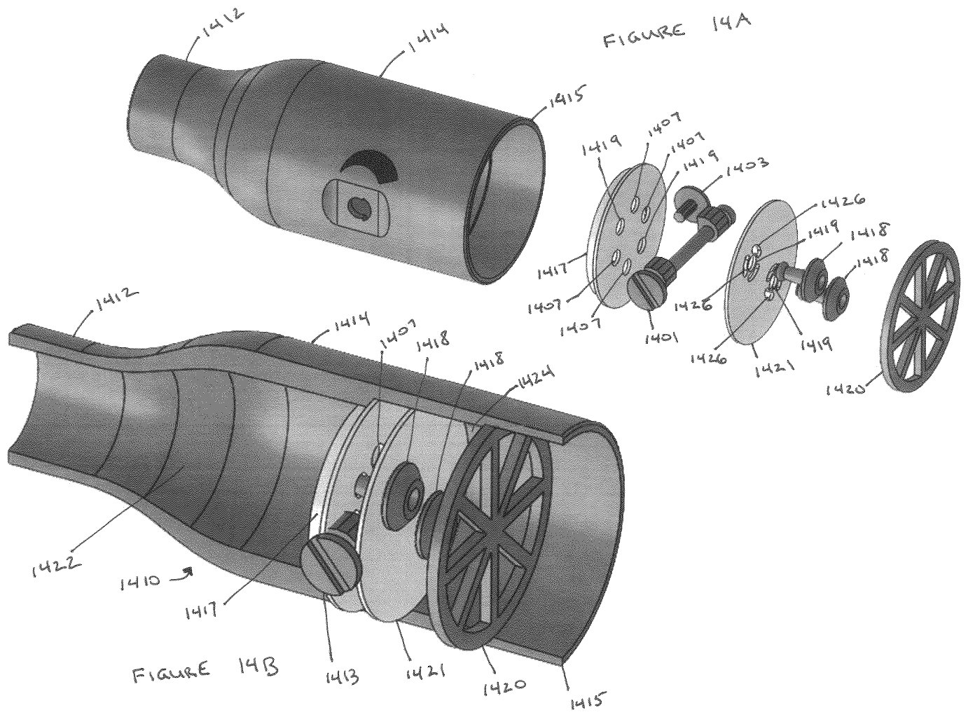

Referring to FIGS. 10A, 10B, 10C, and 10D, another embodiment of a valve assembly of a head and neck exercise apparatus is shown. The valve assembly 1071 may be considered a valve mechanism. The valve assembly 1071 may include a valve assembly housing 1071A, within which there may be a proximal valve assembly plate 1071B, middle valve assembly plate 1071C, proximal valve assembly spring 1071E, proximal valve assembly plate connector 1071G, middle valve assembly plate interface 1071I, and valve assembly end cap 1071L. The valve assembly housing 1071A may define a passageway or central portion 1071J. The proximal valve assembly plate 1071B and valve assembly end cap 1071L may be linked via a connection between the proximal valve assembly plate connector 1071G and the valve assembly end cap 1071L; the connection may occur through the middle valve assembly plate 1071C via the middle valve assembly plate interface 1071I. The connection may be reversible or irreversible.

The proximal valve assembly spring 1071E may maintain the proximal valve assembly plate 1071B in a position at rest that biases the valve assembly 1071 towards an open position. At rest, the proximal valve assembly spring 1071E may contact the middle valve assembly plate 1071C that may be in a stable position within the valve assembly 1071.

In this embodiment, the valve assembly 1071 may be biased towards the open position unless it is closed when the positive pressure in a proximal portion 1022 is greater than a threshold pressure, with all other potential configurations described elsewhere in this application. When the positive pressure in the proximal portion 1022 is greater than a threshold pressure, the proximal valve assembly plate 1071B may be displaced towards the middle valve assembly plate 1071C, and the proximal valve assembly plate 1071B may form a seal against the valve assembly housing 1071A to close the valve assembly 1071 (FIG. 10D). The proximal portion 1022 may therefore be separated from the central portion 1071J.

The threshold pressures of the valve assembly 1071 may be associated with the construction and features of the valve assembly 1071, the components of the valve assembly 1071, and/or properties of other components of the head and neck exercise apparatus.

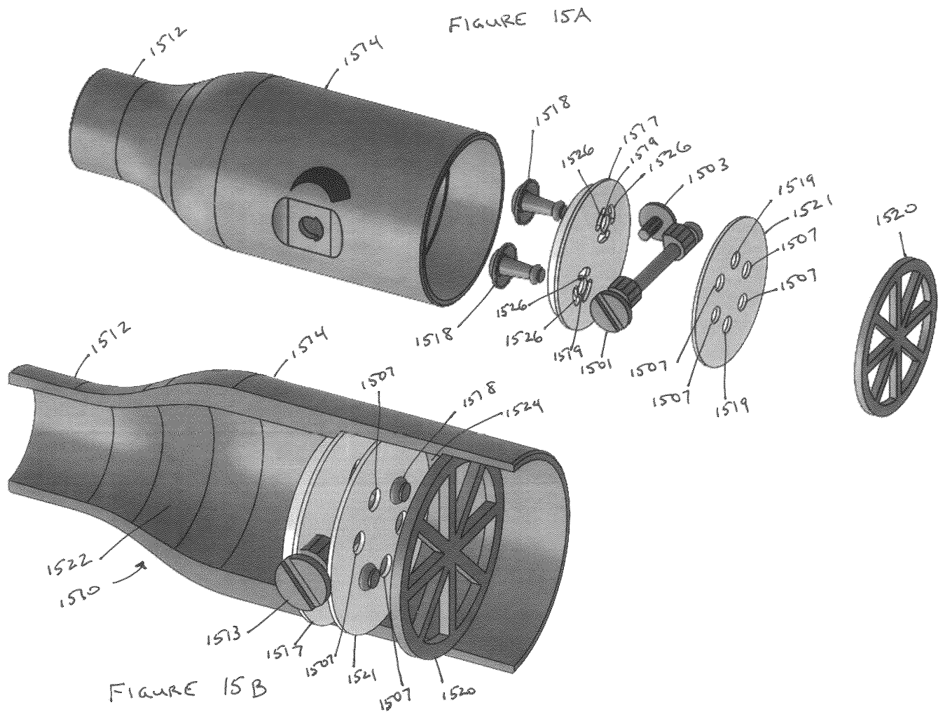

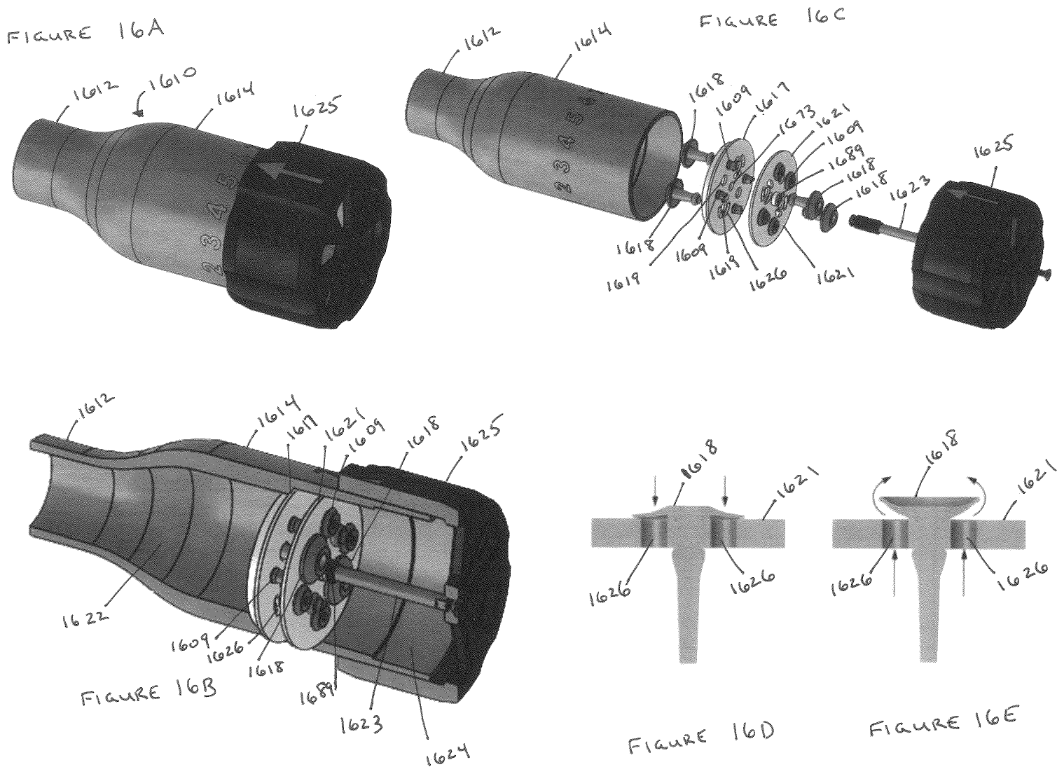

Referring to FIGS. 11A, 11B, 11C, and 11D, another embodiment of a head and neck exercise apparatus 1110 is shown. The head and neck exercise apparatus 1110 may include a proximal interface 1112 and a member 1114. In this embodiment, the member 1114 is generally cylindrically-shaped, as is the proximal interface 1112 that has a similar diameter. The member 1114 may define a passageway or proximal portion 1122 there through. The proximal interface 1112 may be positioned to a user's mouth or connected to another interface component that could permit a user to position the head and neck exercise apparatus 1110 to a part of a user's head and neck.

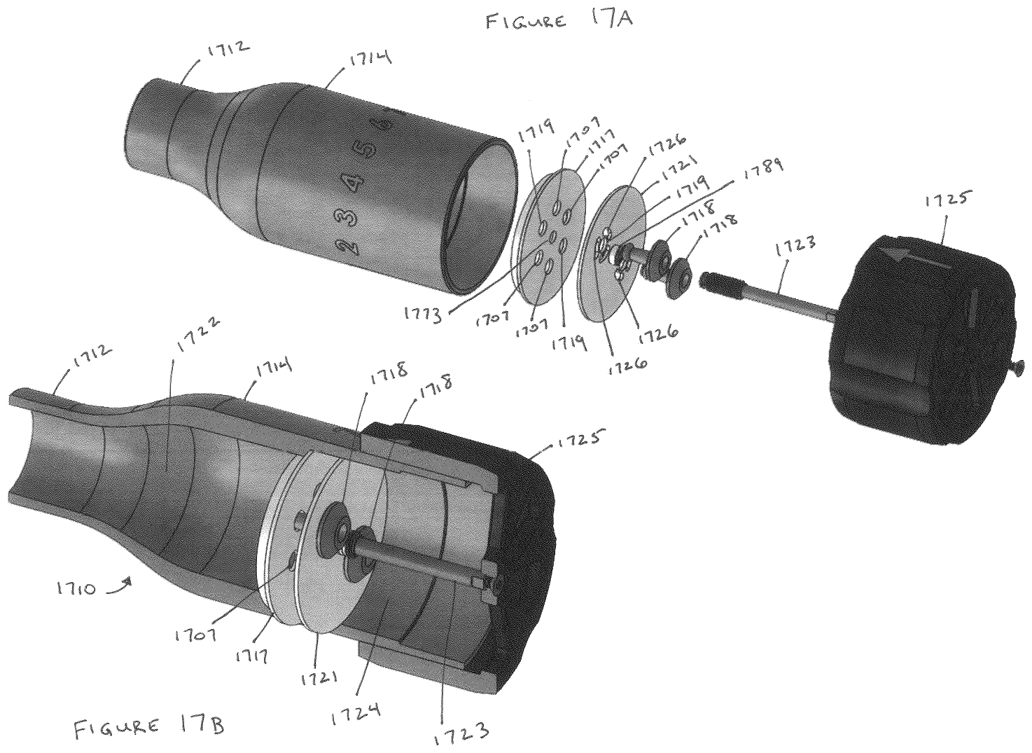

Referring to FIGS. 12A, 12B, and 12C, another embodiment of a head and neck exercise apparatus 1210 is shown. More than one tamper guard 1220 may be positioned to help in shielding the valve plate 1216 and valve mechanisms 1218 from contact.

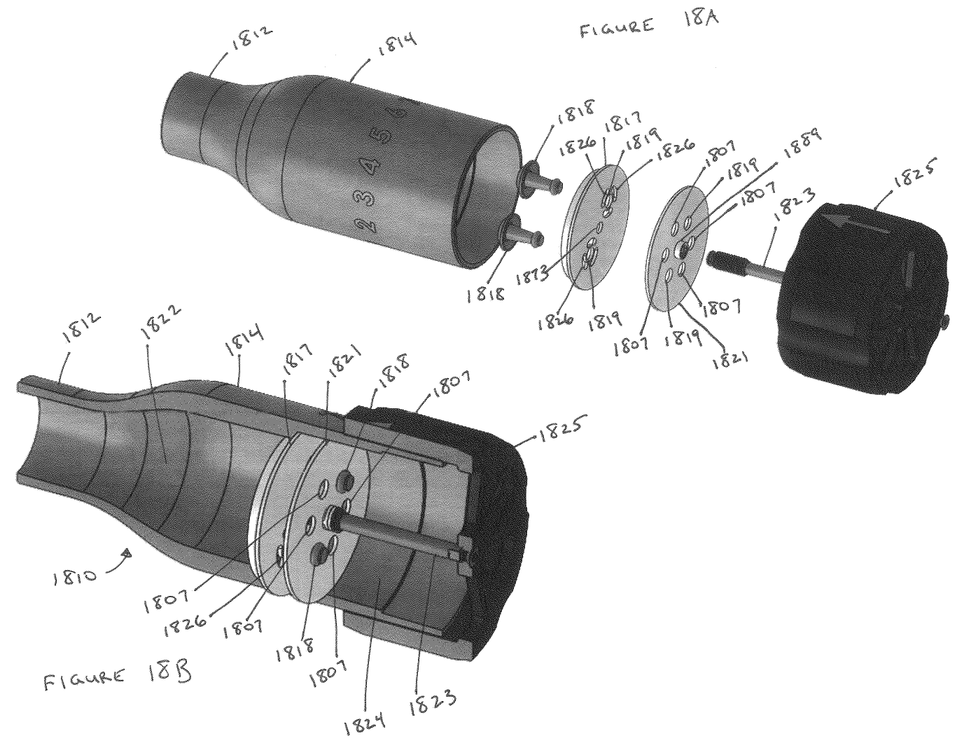

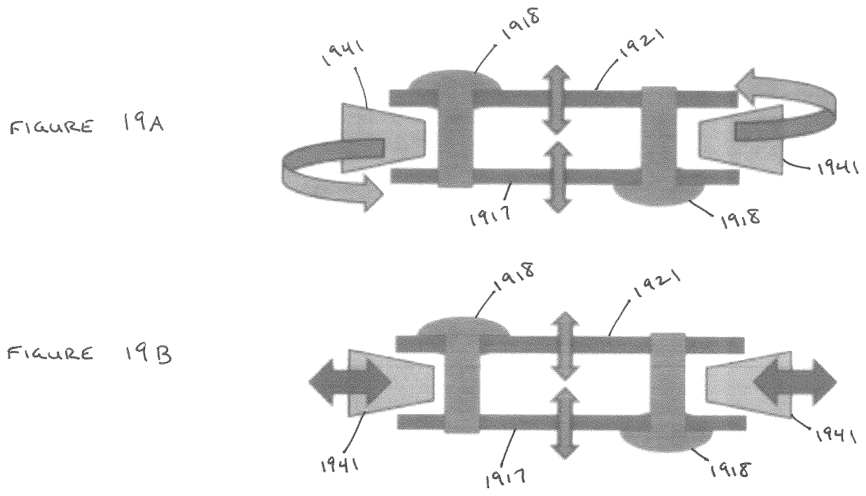

Referring to FIGS. 13A, 13B, 13C, and 13D, another embodiment of a head and neck exercise apparatus 1310 is shown. The head and neck exercise apparatus 1310 may include a proximal interface 1312 and a member 1314. The member 1314 may define a passageway or proximal portion 1322 there through. The proximal interface 1312 may be positioned to a user's mouth or connected to another interface component that could permit a user to position the head and neck exercise apparatus 1310 to a part of a user's head and neck. In this embodiment, the member 1314 also defines a passageway or distal portion 1324 there through. A proximal valve plate 1317, valve plate adjustment mechanism 1313, and distal valve plate 1321 may be operatively arranged with (e.g., disposed within, connected to, etc.) the member 1314. The proximal valve plate 1317 and distal valve plate 1321 may cooperate with at least one valve mechanism 1318 that is positioned within valve stem holes 1319 of both valve plates. In this embodiment, the proximal valve plate 1317 and distal valve plate 1321 each contain four valve stem holes 1319, and each valve mechanism 1318 is positioned within a valve stem hole 1319 in both plates; of the four valve mechanisms 1318, two are oriented in each direction.

Separately, the proximal valve plate 1317 and distal valve plate 1321 may each cooperate with at least one accessory valve mechanism 1309. The at least one accessory valve mechanism 1309 may be oriented to allow airflow through the respective plate when at least one valve mechanism 1318 in the opposite valve plate is open; for example, if the pressure in the proximal portion 1322 is greater than a threshold pressure, airflow may occur from the proximal portion 1322 to the distal portion 1324 by traveling through at least one accessory valve mechanism 1309 in the proximal valve plate 1317 and then through airflow channels 1326 in the distal valve plate 1321 associated with the at least one valve mechanism 1318 that is open. In this embodiment, the threshold pressure of the head and neck exercise apparatus may be primarily defined by the at least one valve mechanism 1318 because the threshold pressure of the accessory valve mechanism 1309 is low, but in other embodiments the accessory valve mechanism 1309 may have any threshold pressure.

The valve plate adjustment mechanism 1313 may be configured to control a separation distance between the proximal valve plate 1317 and distal valve plate 1321. The threshold pressure of the at least one valve mechanism 1318 may be associated with the separation distance between the proximal valve plate 1317 and distal valve plate 1321. The threshold pressure of the at least one valve mechanism 1318 may be associated with the construction and features of the at least one valve mechanism 1318 and/or properties of other components (proximal valve plate 1317, distal valve plate 1321, valve plate adjustment mechanism 1313, member 1314, tamper guard 1320, etc.) of the head and neck exercise apparatus 1310. The valve plate adjustment mechanism 1313 may cooperate with an end stop to limit relative movement of the proximal valve plate 1317 and/or the distal valve plate 1321. In the present embodiment, the valve plate adjustment mechanism 1313 may be rotated in its position to adjust the separation distance. In this embodiment, the proximal valve plate 1317 is fixed in position, while the position of the distal valve plate 1321 is controlled by the valve plate adjustment mechanism 1313; in other embodiments, the proximal valve plate 1317 and distal valve plate 1321 may each be in a fixed or variable position. In this embodiment, the valve plate adjustment mechanism 1313 has two components: the adjustment portion 1301 and adjustment mechanism cap 1303, but the valve plate adjustment mechanism 1313 may be configured as a single component or any number of components. The valve plate adjustment mechanism 1313 may contact the proximal valve plate 1317 and/or the distal valve plate 1321 with flat portions that correspond to specific separation distances (as in this embodiment) or with a rounded contour; the nature of the contact surface may be associated with the means of adjustment of threshold pressure (e.g., rounded contour for gradual adjustment vs. flat portions for discrete levels of threshold pressure, etc.). There may be a biasing means, such as a coil spring, urging at least one of the proximal valve plate 1317 or distal valve plate 1321 towards each other.

Referring to FIGS. 14A and 14B, another embodiment of a head and neck exercise apparatus 1410 is shown. A proximal valve plate 1417, valve plate adjustment mechanism 1413, and distal valve plate 1421 may be operatively arranged with (e.g., disposed within, connected to, etc.) the member 1414. The proximal valve plate 1417 and distal valve plate 1421 may cooperate with at least one valve mechanism 1418 that is positioned within valve stem holes 1419 of both valve plates. In this embodiment, the two valve mechanisms 1418 are oriented in the same direction. In this embodiment airflow may occur through open spaces 1407 in the proximal valve plate 1417. In this embodiment, the head and neck exercise apparatus 1410 may be configured to open the at least one valve mechanism 1418 only with positive pressure in the proximal portion 1422 that exceeds the threshold pressure. In other embodiments, the valve plate may include a single valve mechanism 1418.

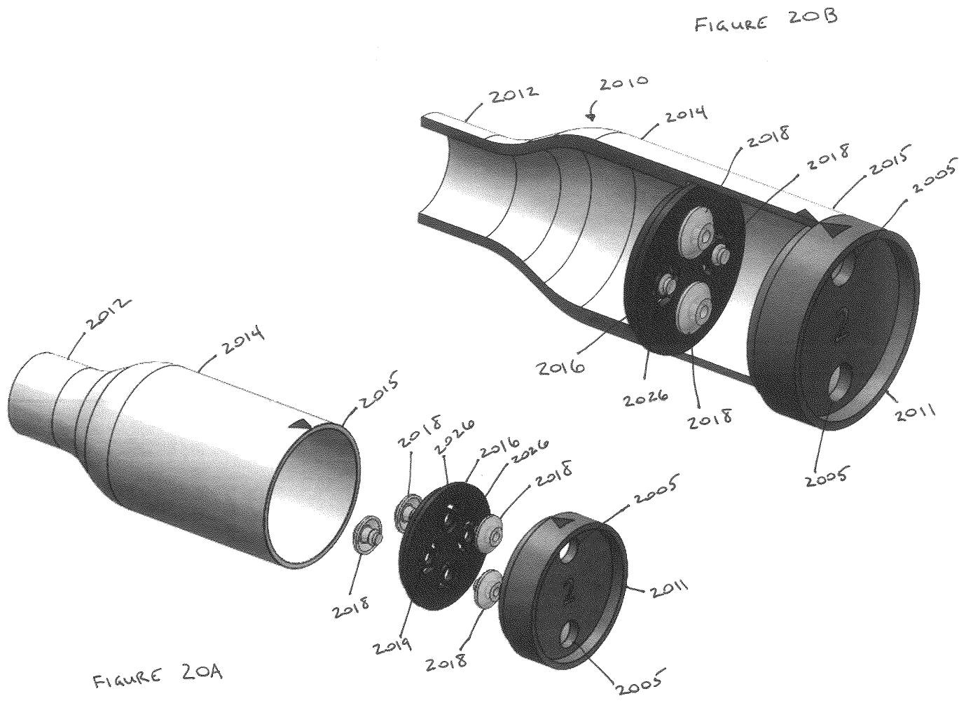

Referring to FIGS. 15A and 15B, another embodiment of a head and neck exercise apparatus 1510 is shown. A proximal valve plate 1517, valve plate adjustment mechanism 1513, and distal valve plate 1521 may be operatively arranged with (e.g., disposed within, connected to, etc.) the member 1514. The proximal valve plate 1517 and distal valve plate 1521 may cooperate with at least one valve mechanism 1518 that is positioned within valve stem holes 1519 of both valve plates. In this embodiment, the two valve mechanisms 1518 are oriented in the same direction. In this embodiment airflow may occur through open spaces 1507 in the distal valve plate 1521. In this embodiment, the head and neck exercise apparatus 1510 may be configured to open the at least one valve mechanism 1518 only with negative pressure in the proximal portion 1522 that exceeds the threshold pressure. In other embodiments, the valve plate may include a single valve mechanism 1518.

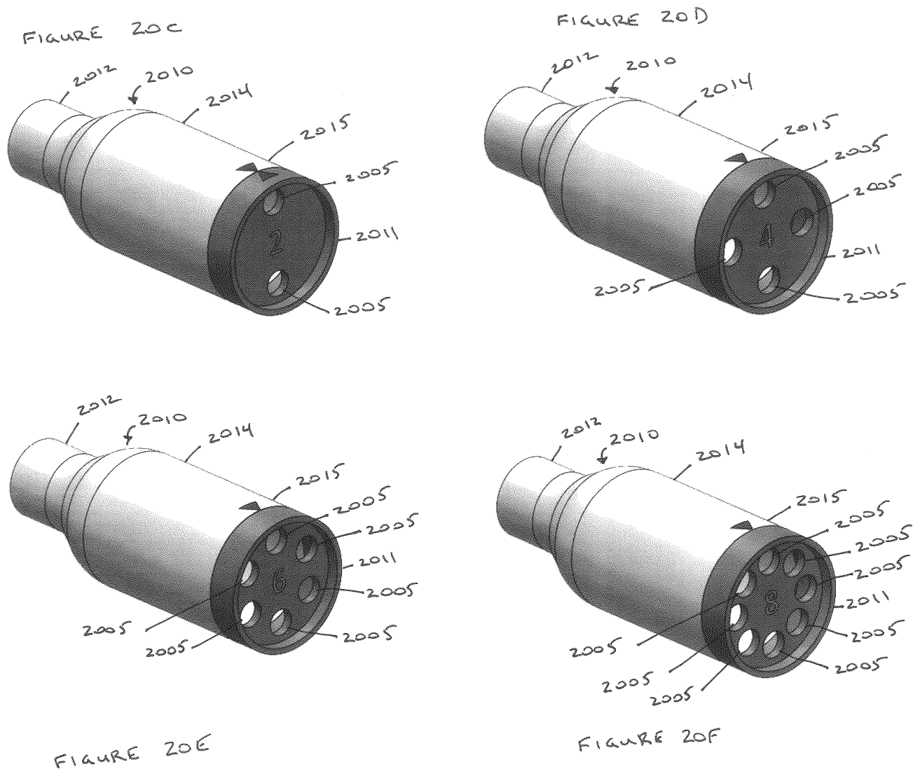

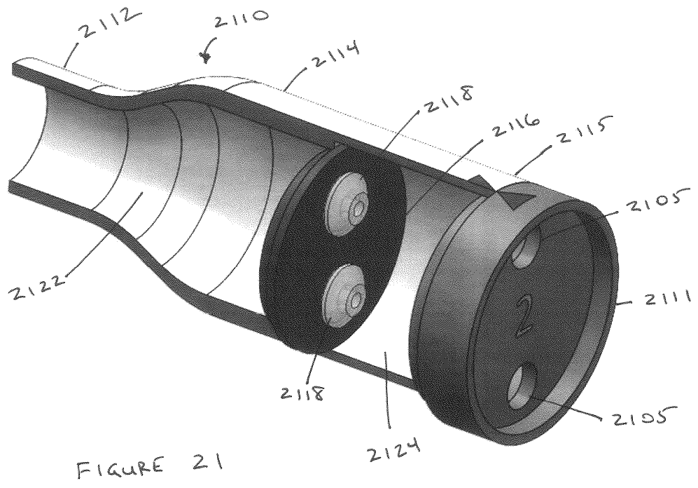

Referring to FIGS. 16A, 16B, 16C, 16D, and 16E, another embodiment of a head and neck exercise apparatus 1610 is shown. The head and neck exercise apparatus 1610 may include a proximal interface 1612 and a member 1614. The member 1614 may define a passageway or proximal portion 1622 there through. The member 1614 also may define a passageway or distal portion 1624 there through.

A proximal valve plate 1617, distal valve plate 1621, and valve plate adjustment element 1623 may be operatively arranged with (e.g., disposed within, connected to, etc.) the member 1614. The proximal valve plate 1617 and distal valve plate 1621 may separate the proximal portion 1622 and distal portion 1624 when all valve mechanisms 1618 are in the closed position. A valve plate adjustment element 1623 may be configured to control a separation distance between the proximal valve plate 1617 and distal valve plate 1621. In this embodiment, the proximal valve plate is in a fixed position while the distal valve plate 1621 is in a variable position, but other permutations are possible. In this embodiment, the plate adjustment element 1623 may cooperate with a proximal valve plate adjustment interface 1673 and distal plate adjustment interface 1689; the cooperation with the proximal valve plate adjustment interface 1673 may stabilize the plate adjustment element 1623, while the cooperation with the distal plate adjustment interface 1689 may enable control of the plate adjustment element 1623 to control the position of the distal valve plate 1621. The valve plate adjustment element 1623 may be configured to have discrete settings associated with specific separation distances or enable continuous adjustment. The valve plate adjustment element 1623 may cooperate with an end stop to limit relative movement of the proximal valve plate 1617 and/or the distal valve plate 1621. In the present embodiment, the valve plate adjustment element 1623 adjusts the position of the distal valve plate 1621, but in other embodiments it could adjust the position of the proximal valve plate 1617 and/or distal valve plate 1621. In the present embodiment, the valve plate adjustment element 1623 is connected to an end cap 1625 that enables a user to select settings that correspond to separation distances. The end cap 1625 also may be configured to include features of a tamper guard, as in the present embodiment, or there may be a separate tamper guard.

The threshold pressure of the at least one valve mechanism 1618 may be associated with the separation distance between the proximal valve plate 1617 and distal valve plate 1621. The threshold pressure of the at least one valve mechanism 1618 may be associated with the construction and features of the at least one valve mechanism 1618 and/or properties of other components (proximal valve plate 1617, distal valve plate 1621, valve plate adjustment element 1623, member 1614, tamper guard 1620, etc.) of the head and neck exercise apparatus 1610.

Separately, the proximal valve plate 1617 and distal valve plate 1621 may each cooperate with at least one accessory valve mechanism 1609. The at least one accessory valve mechanism 1609 may be oriented to allow airflow through the respective plate when at least one valve mechanism 1618 in the opposite valve plate is open; for example, if the pressure in the proximal portion 1622 is greater than a threshold pressure, airflow may occur from the proximal portion 1622 to the distal portion 1624 by traveling through at least one accessory valve mechanism 1609 in the proximal valve plate 1617 and then through airflow channels 1626 in the distal valve plate 1621 associated with the at least one valve mechanism 1618 that is open. In this embodiment, the threshold pressure of the head and neck exercise apparatus is primarily defined by the at least one valve mechanism 1618 because the threshold pressure of the accessory valve mechanism 1609 is low, but in other embodiments the accessory valve mechanism 1609 may have any threshold pressure.

In this embodiment, each valve mechanism 1618 is oriented in a specific direction, such that it will only permit airflow in a specific direction. For a valve mechanism 1618 oriented to permit airflow from the proximal portion 1622 to the distal portion 1624, no airflow will occur from the opposite direction (FIG. 16D). However, when the positive pressure in the proximal portion 1622 is greater than the threshold pressure, this may displace a portion of the valve mechanism 1018 to open the valve mechanism 1618 (FIG. 16E); the proximal portion 1622 will be fluidly connected to the distal portion 1624, allowing airflow from the proximal portion 1622 to the distal portion 1624 though an airflow channel 1626.

Referring to FIGS. 17A and 17B, another embodiment of a head and neck exercise apparatus 1710 is shown. A proximal valve plate 1717, valve plate adjustment mechanism 1713, and distal valve plate 1721 may be operatively arranged with (e.g., disposed within, connected to, etc.) the member 1714. The proximal valve plate 1717 and distal valve plate 1721 may cooperate with at least one valve mechanism 1718 that is positioned within valve stem holes 1719 of both valve plates. In this embodiment, the two valve mechanisms 1718 are oriented in the same direction. In this embodiment airflow may occur through open spaces 1707 in the proximal valve plate 1717. In this embodiment, the head and neck exercise apparatus 1710 may be configured to open the at least one valve mechanism 1718 only with positive pressure in the proximal portion 1722 that exceeds the threshold pressure. In other embodiments, the valve plate may include a single valve mechanism 1718.

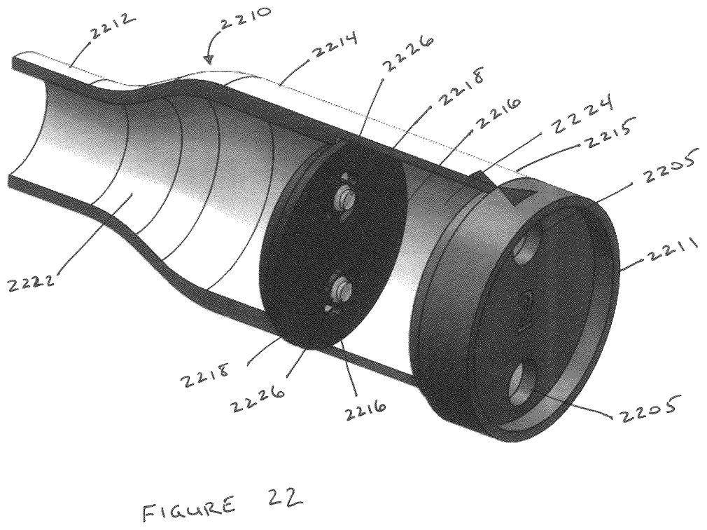

Referring to FIGS. 18A and 18B, another embodiment of a head and neck exercise apparatus 1810 is shown. A proximal valve plate 1817, valve plate adjustment mechanism 1813, and distal valve plate 1821 may be operatively arranged with (e.g., disposed within, connected to, etc.) the member 1814. The proximal valve plate 1817 and distal valve plate 1821 may cooperate with at least one valve mechanism 1818 that is positioned within valve stem holes 1819 of both valve plates. In this embodiment, the two valve mechanisms 1818 are oriented in the same direction. In this embodiment airflow may occur through open spaces 1807 in the distal valve plate 1821. In this embodiment, the head and neck exercise apparatus 1810 may be configured to open the at least one valve mechanism 1818 only with negative pressure in the proximal portion 1822 that exceeds the threshold pressure. In other embodiments, the valve plate may include a single valve mechanism 1818.

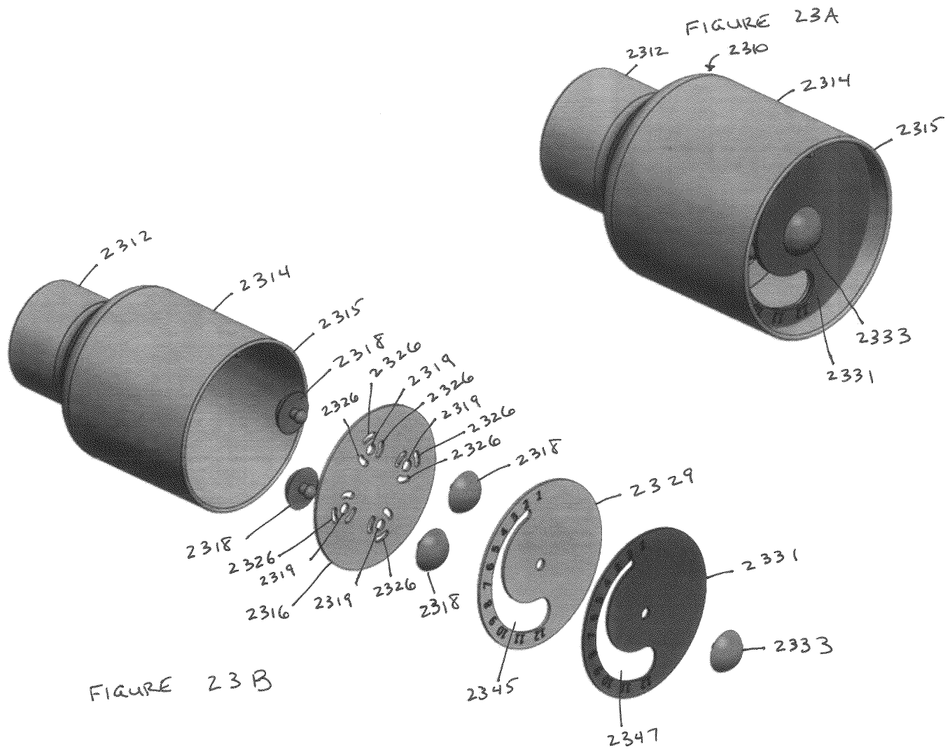

Referring to FIGS. 19A and 19B, another embodiment of a portion of a head and neck exercise apparatus is shown. A mechanism for adjusting the relative position of valve plates is shown. At least one plate adjustment block 1941 may be positioned between a proximal valve plate 1917 and distal valve plate 1921. The present embodiment includes a tapered shape to the two plate adjustment blocks 1941, such that an adjustment of the position of the plate adjustment blocks 1941 relative to the proximal valve plate 1917 and distal valve plate 1921 will vary the separation distance between the latter two. In this embodiment, the two plate adjustment blocks 1941 change position in the same general pattern, but other permutations are possible. Other shapes or configurations of the at least one plate adjustment block 1941 are possible. This adjustment of the position of the plate adjustment blocks 1941 may occur with rotation (FIG. 19A), movement in a linear direction (FIG. 19B), and/or another type of movement.

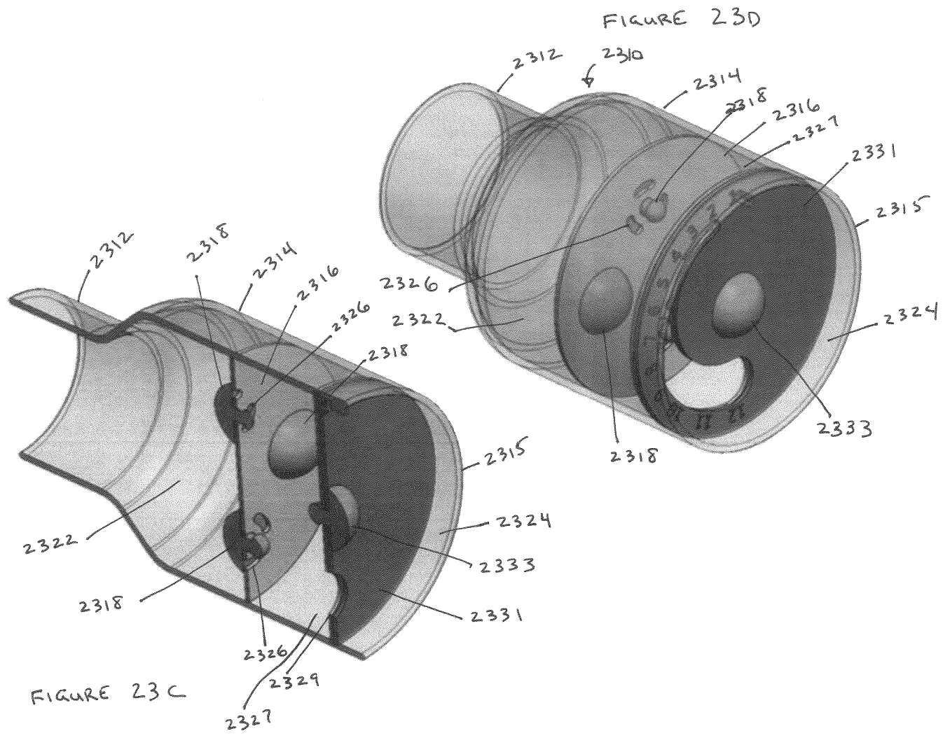

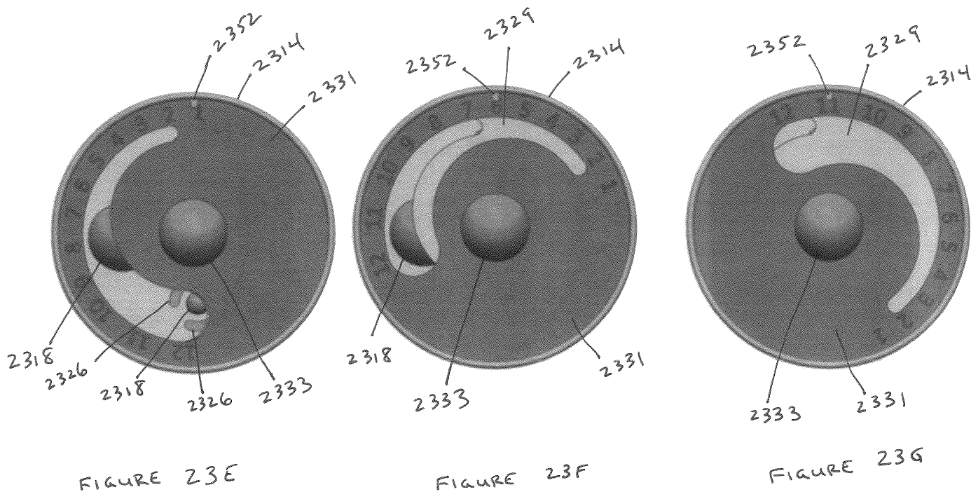

Referring to FIGS. 20A, 20B, 20C, 20D, 20E, and 20F, another embodiment of a head and neck exercise apparatus 2010 is shown. The head and neck exercise apparatus 2010 may include an airflow resistance element 2011 that may reversibly or irreversibly cooperate with a member 2014 at a distal interface 2015. A threshold pressure of the head and neck exercise apparatus 2010 may therefore additionally depend on the properties of the airflow resistance element 2011. In this embodiment, the airflow resistance element 2011 cooperates with the distal interface 2015 and includes multiple airflow resistance spaces 2005 of similar shape and dimensions. In other embodiments, the airflow resistance element 2011 may be incorporated in any configuration and include any number of airflow resistance spaces 2005 or other variations (e.g., shape, dimension, position, etc.). In this embodiment, the airflow resistance mechanism is controlled by a physical means, but in other embodiments the control may be magnetic, electrical, or electronic.

A coupling mechanism, such as a locking or snap mechanism, may facilitate the cooperation between the member 2014 and airflow resistance element 2011. A reversible cooperation may allow the mask interface to cooperate with the member 2014 of different head and neck exercise apparatuses 2010 that may have different properties. A reversible cooperation may also allow the member 2014 to cooperate with different airflow resistance elements 2011 (as in FIGS. 20C-20F), and these could be considered different head and neck exercise apparatuses. The airflow resistance elements 2011 may also be adjustable. Adjustment or substitution of an airflow resistance element 2011 may be considered a modification.

Cooperation of the member 2014 with different airflow resistance elements 2011 could enable a user to perform head and neck exercises for sleep disordered breathing using a range of threshold pressures that could be desirable. An exercise program may incorporate exercises that require different threshold pressures, and the use of head and neck exercise apparatuses 2010 with a range of threshold pressures or multiple apparatuses would reduce the need for adjustment or calibration of any single head and neck exercise apparatus 2010. Adjustment and calibration can introduce cost, complexity, and expenditure of time and effort, any of which may be undesirable.

Referring to FIG. 21, another embodiment of a head and neck exercise apparatus 2110 is shown. The valve plate 2116 may cooperate with at least one valve mechanism 2118. In this embodiment, the two valve mechanisms 2118 are oriented in the same direction. In this embodiment, the head and neck exercise apparatus 2110 may be configured to open the at least one valve mechanism 2118 only with positive pressure in the proximal portion 2122 that exceeds the threshold pressure. In other embodiments, the valve plate may include a single valve mechanism 2118.

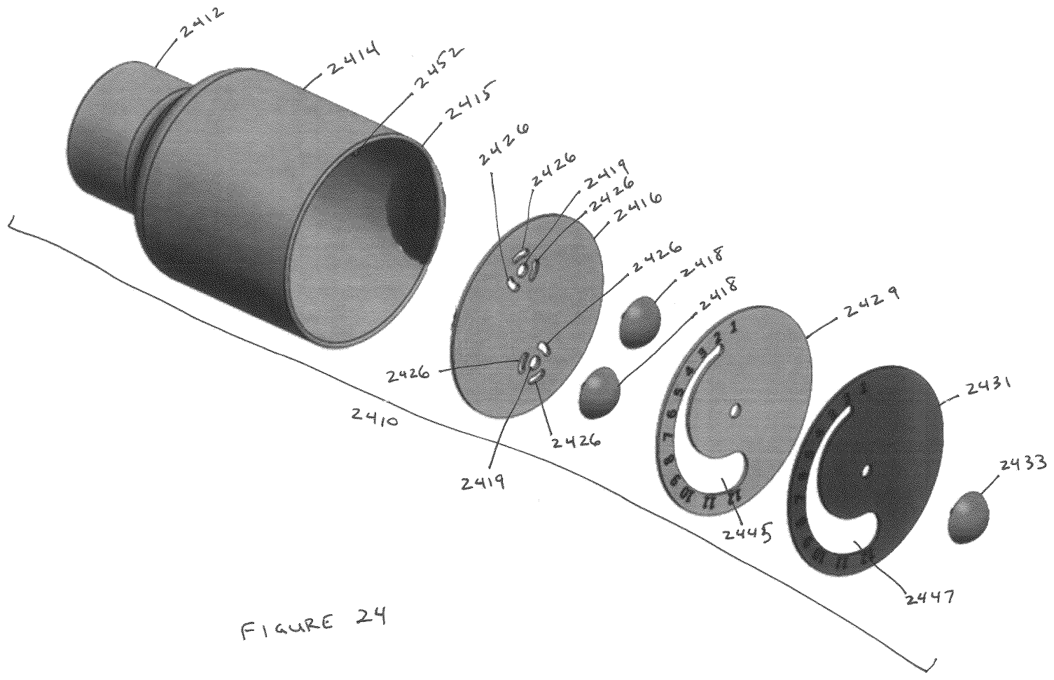

Referring to FIG. 22, another embodiment of a head and neck exercise apparatus 2210 is shown. The valve plate 2216 may cooperate with at least one valve mechanism 2218. In this embodiment, the two valve mechanisms 2218 are oriented in the same direction. In this embodiment, the head and neck exercise apparatus 2210 may be configured to open the at least one valve mechanism 2218 only with negative pressure in the proximal portion 2222 that exceeds the threshold pressure. In other embodiments, the valve plate may include a single valve mechanism 2218.

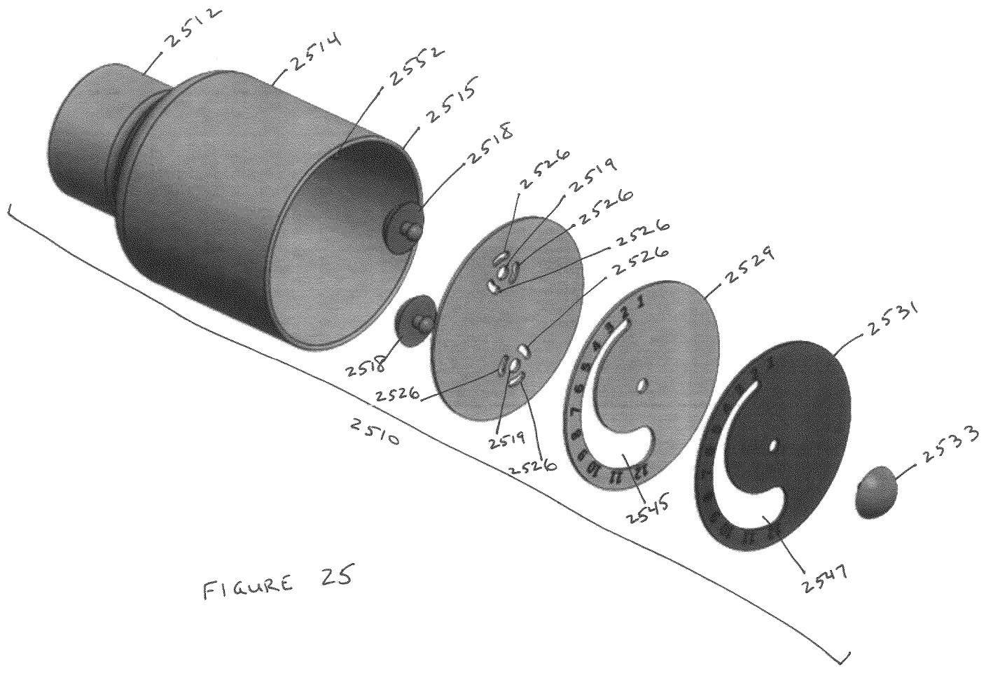

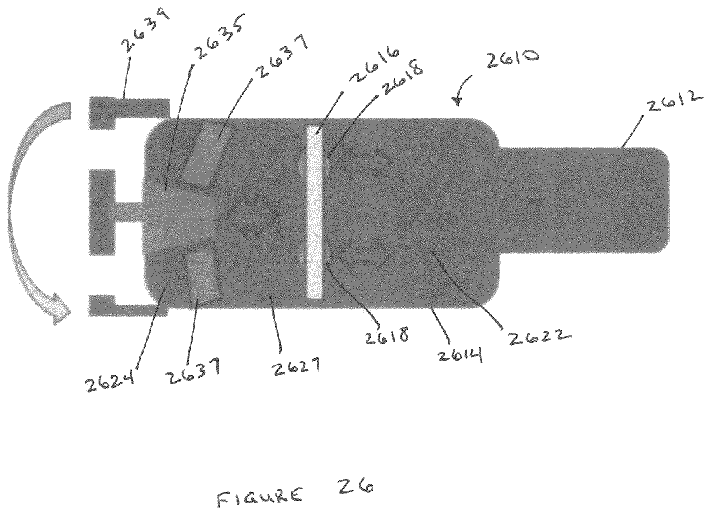

Referring to FIGS. 23A, 23B, 23C, 23D, 23E, 23F, and 23G, another embodiment of a head and neck exercise apparatus 2310 is shown. The head and neck exercise apparatus 2310 may include a proximal interface 2312 and a member 2314. The member 2314 may define a passageway or proximal portion 2322 there through. The proximal interface 2312 may be positioned to a user's mouth or connected to another interface component that could permit a user to position the head and neck exercise apparatus 2310 to a part of a user's head and neck. In this embodiment, the member 2314 also defines a passageway or central portion 2327 there through. In this embodiment, the member 2314 also defines a passageway or distal portion 2324 there through.

A valve plate 2316 may be operatively arranged with (e.g., disposed within, connected to, etc.) the member 2314. The valve plate may cooperate with at least one valve mechanism 2318 that is positioned within a valve stem hole 2319. In this embodiment, the valve plate 2316 contains four valve stem holes 2319, through which a valve mechanism 2318 is positioned in each one; of the four valve mechanisms 2318, two are oriented in each direction. The valve plate 2316 may separate the proximal portion 2322 and central portion 2327 when all valve mechanisms 2318 are in the closed position.

A proximal resistance plate 2329 and distal resistance plate 2331 may be operatively arranged with (e.g., disposed within, connected to, etc.) the member 2314. The proximal resistance plate 2329 and distal resistance plate 2331 may collectively function as an airflow resistance mechanism. The proximal resistance plate 2329 may define at least one proximal resistance plate aperture 2345. The distal resistance plate 2329 may define at least one distal resistance plate aperture 2347. The size and shape of the at least one proximal resistance plate aperture 2345 and the at least one distal resistance plate aperture 2347 may be substantially the same, as in the present embodiment, but the sizes and/or shapes may differ. At least one of the proximal resistance plate 2329 and distal resistance plate 2331 may be moveable. In this embodiment, the proximal resistance plate 2329 and distal resistance plate 2331 cooperate with a resistance plate knob 2333, with the distal resistance plate 2331 configured to rotate as a means of adjusting the relative position of the proximal resistance plate 2329 and distal resistance plate 2331. The resistance plate knob 2333 may be configured to adjust the position of the distal resistance plate 2331, but other configurations are possible. A resistance indicator marker 2352 may assist the user in selection of a desired position of at least one of the proximal resistance plate 2329 and the distal resistance plate 2331. The proximal resistance plate 2329 and/or distal resistance plate 2331 may cooperate with an end stop to limit relative movement of the proximal resistance plate 2329 and/or distal resistance plate 2331. There may be a biasing means, such as a coil spring, urging at least one of the proximal resistance plate 2329 and/or distal resistance plate 2331 towards each other. In another embodiment, a biasing means could include placement of an additional resistance plate, similar to the proximal resistance plate 2329, adjacent to the distal resistance plate 2331 to sandwich the distal resistance plate 2331 between the proximal resistance plate 2329 and the additional resistance plate.

The relative position of the proximal resistance plate 2329 and distal resistance plate 2331 (collectively, an airflow resistance mechanism) may provide another mechanism to modify the threshold pressure for airflow through the head and neck exercise apparatus 2310. When the proximal resistance plate 2329 and distal resistance plate 2331 are oriented such that at least one proximal resistance plate aperture 2345 and at least one distal resistance plate aperture 2347 are not in registration, the proximal resistance plate 2329 and distal resistance plate 2331 may separate the central portion 2327 and distal portion 2324. When at least one proximal resistance plate aperture 2345 and at least one distal resistance plate aperture 2347 are in registration or partial registration, airflow may occur through the at least one common aperture, and the change in threshold pressure may be associated with the area of the total area of the at least one common aperture. The proximal resistance plate 2329 and/or distal resistance plate 2331 may be configured to have discrete settings associated with specific areas of at least one common aperture or enable continuous adjustment. The head and neck exercise apparatus 2310 may be configured to permit substitution of any or all components of the airflow resistance mechanism. Adjustment or substitution of any or all components of the airflow resistance mechanism may modify the airflow resistance.

The threshold pressure of the at least one valve mechanism 2318 may also be associated with the construction and features of the at least one valve mechanism 2318, airflow resistance mechanism, and/or properties of other components (valve plate 2316, member 2314, proximal resistance plate 2329, proximal resistance plate aperture 2345, distal resistance plate 2331, distal resistance plate aperture 2347, etc.) of the head and neck exercise apparatus 2310.

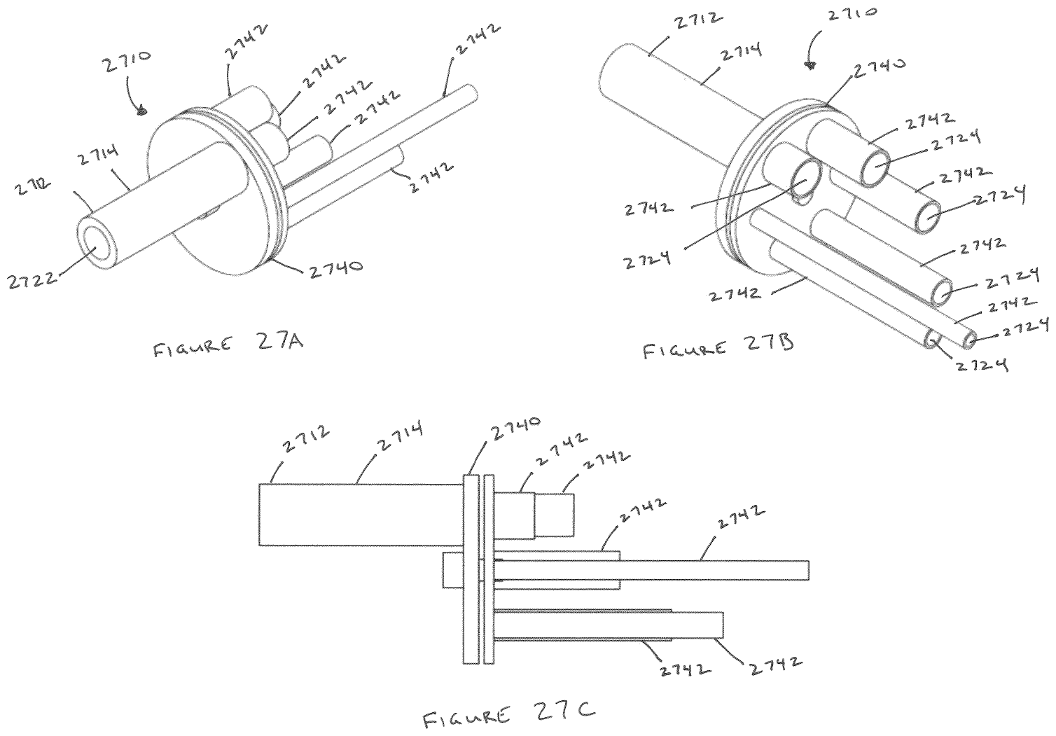

Referring to FIG. 24, another embodiment of a head and neck exercise apparatus 2410 is shown. The valve plate 2416 may cooperate with at least one valve mechanism 2418. In this embodiment, the two valve mechanisms 2418 are oriented in the same direction. In this embodiment, the head and neck exercise apparatus 2410 may be configured to open the at least one valve mechanism 2418 only with positive pressure in the proximal portion 2422 that exceeds the threshold pressure. In other embodiments, the valve plate may include a single valve mechanism 2418.

Referring to FIG. 25, another embodiment of a head and neck exercise apparatus 2510 is shown. The valve plate 2516 may cooperate with at least one valve mechanism 2518. In this embodiment, the two valve mechanisms 2518 are oriented in the same direction. In this embodiment, the head and neck exercise apparatus 2510 may be configured to open the at least one valve mechanism 2518 only with negative pressure in the proximal portion 2522 that exceeds the threshold pressure. In other embodiments, the valve plate may include a single valve mechanism 2518.

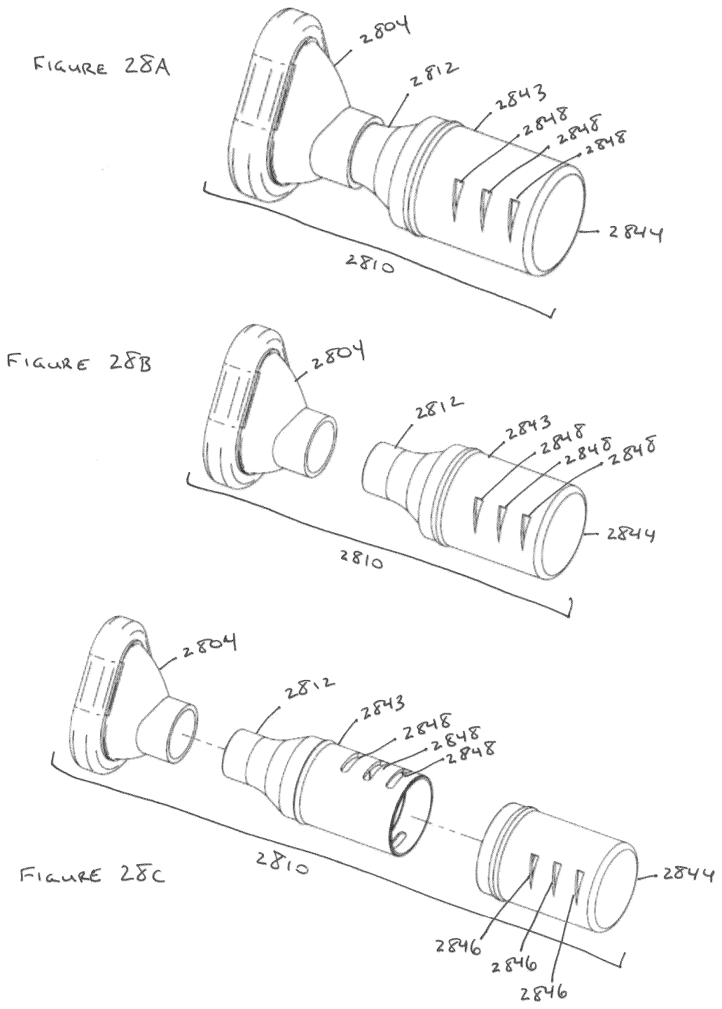

Referring to FIG. 26, another embodiment of a head and neck exercise apparatus 2610 is shown. The head and neck exercise apparatus 2610 may include a proximal interface 2612 and a member 2614. The member 2614 may define a passageway or proximal portion 2622 there through. In this embodiment, the member 2614 also defines a passageway or central portion 2627 there through. In this embodiment, the member 2614 also defines a passageway or distal portion 2624 there through. A valve plate 2616 may be operatively arranged with (e.g., disposed within, connected to, etc.) the member 2614. The valve plate may cooperate with at least one valve mechanism 2618. The valve plate 2616 may separate the proximal portion 2622 and central portion 2627 when all valve mechanisms 2618 are in the closed position.

The head and neck exercise apparatus 2610 may include an aperture block 2635 and aperture plate 2637. The aperture block 2635 may be configured with an aperture block control 2639, such that the aperture block control 2639 may move the aperture block 2635 relative to the aperture plate 2637. The relative position of the aperture block 2635 and aperture plate 2637 may determine the characteristics (e.g., area, shape, etc.) of the space between them; this may function as an airflow resistance mechanism and provide another mechanism to modify the threshold pressures for airflow through the head and neck exercise apparatus 2610 (e.g., from the distal portion 2624 to the proximal portion 2622 with negative pressure in the proximal portion 2622). The head and neck exercise apparatus 2610 may be configured with a mechanism to determine the characteristics of the space between the aperture block 2635 and aperture plate 2637, such as an optical or magnetic encoder.

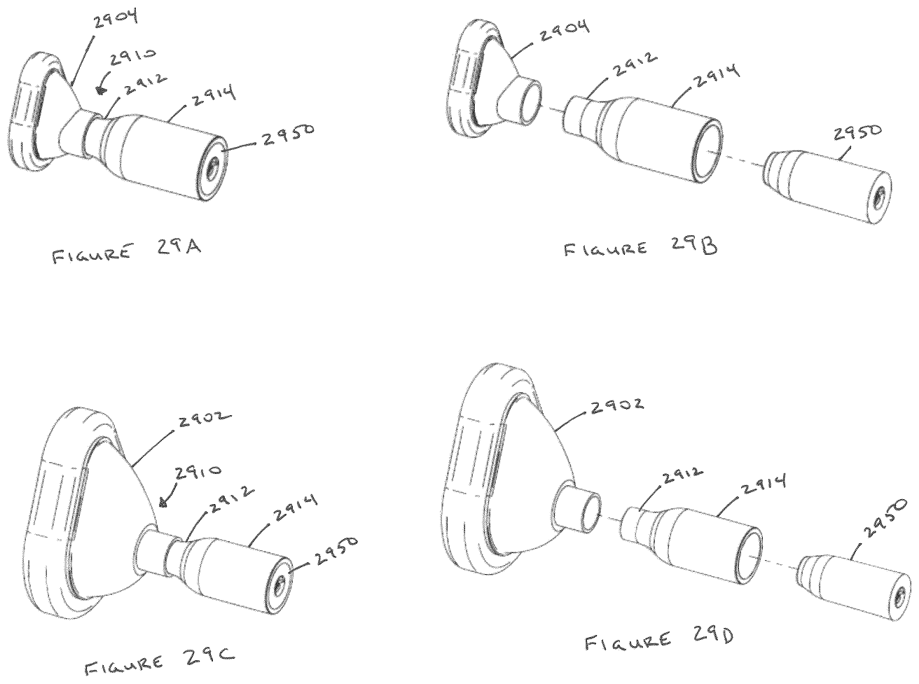

Referring to FIGS. 27A, 27B, and 27C, another embodiment of a head and neck exercise apparatus 2710 is shown. The head and neck exercise apparatus 2710 may include a proximal interface 2712 and member 2714. The member 2714 may define a proximal portion 2722. A resistance mechanism holder 2740 may house at least two resistance members 2742, each of which may define a distal portion 2724. The resistance mechanism holder 2740 may be configured to allow positioning of different resistance members 2742, such that the proximal portion 2722 is in fluid communication with a distal portion 2724. The resistance members 2742 may have different airflow resistance properties related to their dimensions (e.g., diameter and length) or other characteristics that distinguish them as airflow resistance mechanisms. The proximal interface 2712, proximal portion 2722, resistance mechanism holder 2740, resistance members 2742, and/or distal portions 2724 may be configured to incorporate a valve mechanism that may alter resistance to airflow.

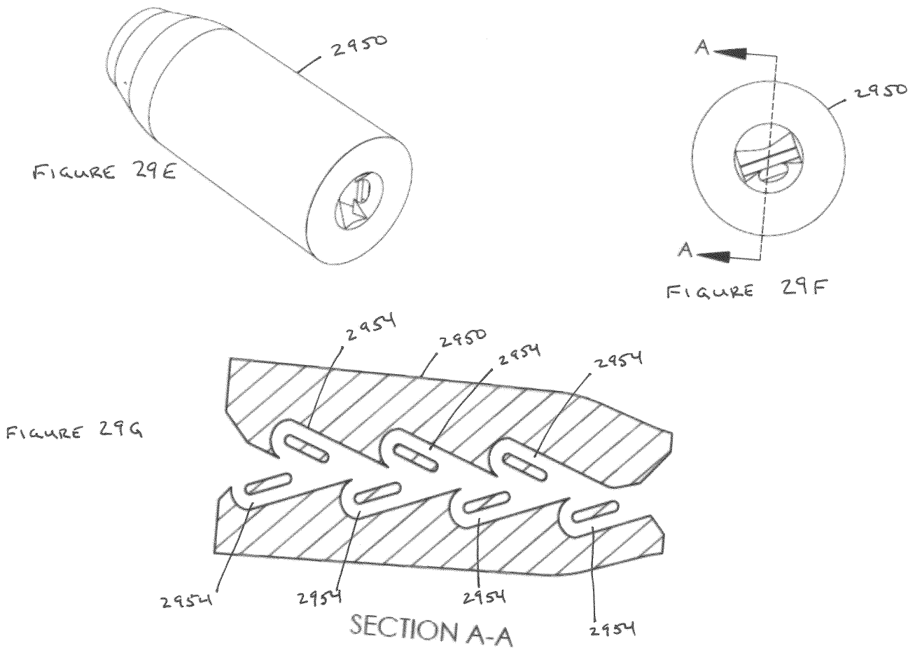

Referring to FIGS. 28A, 28B, and 28C, other embodiments of head and neck exercise apparatuses 2810 are shown. The head and neck exercise apparatus 2810 may include an outer vent member 2843 and inner vent member 2844. Each head and neck exercise apparatus 2810 may include a mask interface that connects to the outer vent member 2843 at a proximal interface 2812. The mask interface may be a nasal mask interface 2804. The proximal interface 2812 may define a passageway or proximal portion there through. The inner vent member 2844 may define a passageway or distal portion there through. The outer vent member 2843 may contain at least one outer airflow vent 2848, and the inner vent member 2844 may contain at least one inner airflow vent 2846.

The outer vent member 2843 and inner vent member 2844 may collectively function as an airflow resistance mechanism. The relative position of the outer vent member 2843 and inner vent member 2844 may determine the resistance to airflow of the head and neck exercise apparatus 2810. When at least one outer vent 2848 and at least one inner airflow vent 2846 are in registration or partial registration, airflow may occur through the at least one common aperture, and the resistance to airflow may be associated with area of the at least one common aperture. The resistance to airflow through the head and neck exercise apparatus 2810 may also be associated with the properties of the outer vent member 2843 (e.g., dimensions, etc.), inner vent member 2844 (e.g., dimensions, etc.), at least one outer airflow vent 2848 (e.g., dimensions, thickness, etc.), and/or at least one inner airflow vent 2846 (e.g., dimensions, thickness, etc.). The resistance to airflow through the head and neck exercise apparatus may also be associated with properties of other components of the head and neck exercise apparatus 2810 (proximal interface 2812, etc.). The proximal interface 2812, proximal portion, inner vent member 2844, distal portion, outer vent member 2843, at least one inner airflow vent 2846, and/or at least one outer airflow vent 2848 may be configured to incorporate a valve mechanism that may alter resistance to airflow. Adjustment of the relative position of the outer vent member 2843 and inner vent member 2844 or adjustment or substitution of any or all components of the head and neck exercise apparatus 2810 may be considered a modification.

A head and neck exercise system may include at least one head and neck exercise apparatus with an airflow resistance mechanism that is adjustable or at least two head and neck exercise apparatuses with airflow resistance mechanisms that may be adjustable or not adjustable. The head and neck exercise system could be designed to provide a range of airflow resistances for the performance of head and neck exercises.

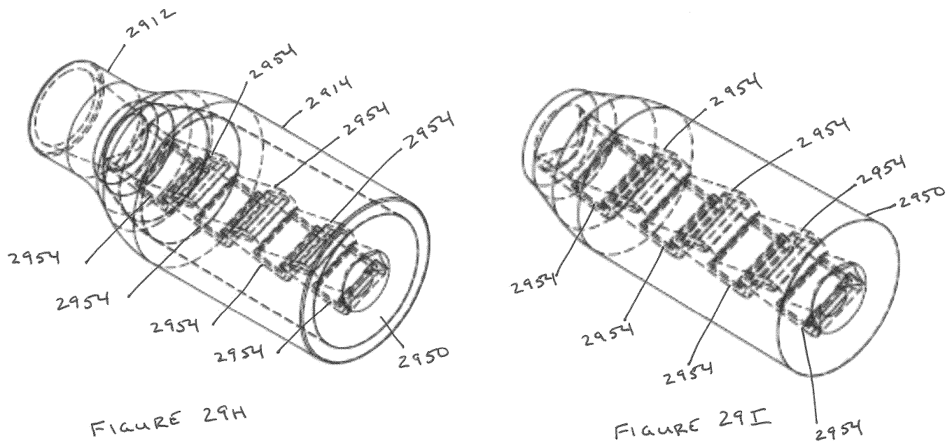

Referring to FIGS. 29A, 29B, 29C, 29D, 29E, 29F, 29G, 29H, and 29I, other embodiments of head and neck exercise apparatuses 2910 are shown. The head and neck exercise apparatuses 2910 may include a mask interface that connects to a member 2914 at the proximal interface 2912. The mask interface may be a nasal mask interface 2904 or an oronasal mask interface 2902. The head and neck exercise apparatus 2910 may include a member 2914 and inner valve member 2950. The inner valve member 2950 may include an airflow resistance mechanism. The inner valve member 2950 may include a valve mechanism. The resistance properties of the head and neck exercise apparatus 2910 may be modified by exchange of the inner valve member 2950 for another inner valve member 2950 with different properties. In this embodiment, the inner valve member 2950 includes a Tesla valve, or valvular conduit. In other embodiments, the inner valve member 2950 may include at least one active valve and/or at least one passive valve. In this embodiment, the resistance to airflow through the inner valve member 2950 may be related to the orientation, position, size, and number of the at least one valve segment 2954. The mechanism of Tesla valves is known in the prior art; airflow through the inner valve member 2950 in the so-called low flow direction (from right to left in FIG. 29G) will produce division of the airflow into two streams, with one stream passing through the valve segment 2954 to create a backflow that increases the resistance to airflow in the low flow direction. Exchange of the inner valve member 2950 or adjustment or exchange of any or all components of the head and neck exercise apparatus 2910 may modify the resistance properties of the head and neck exercise apparatus 2910.

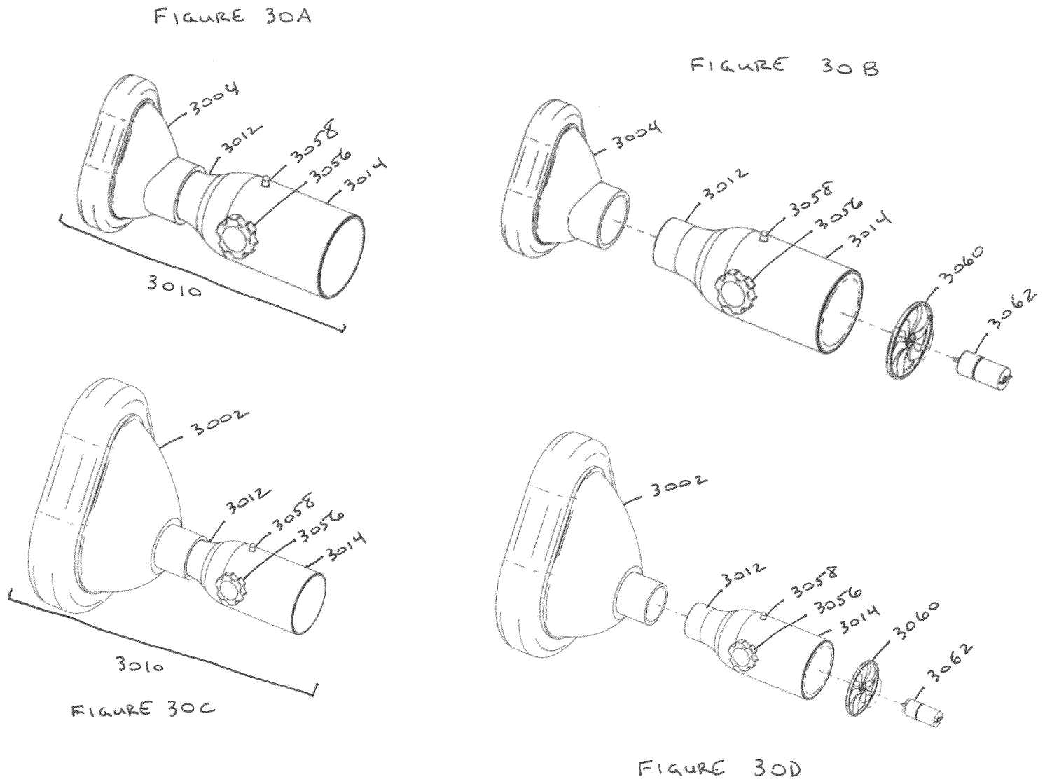

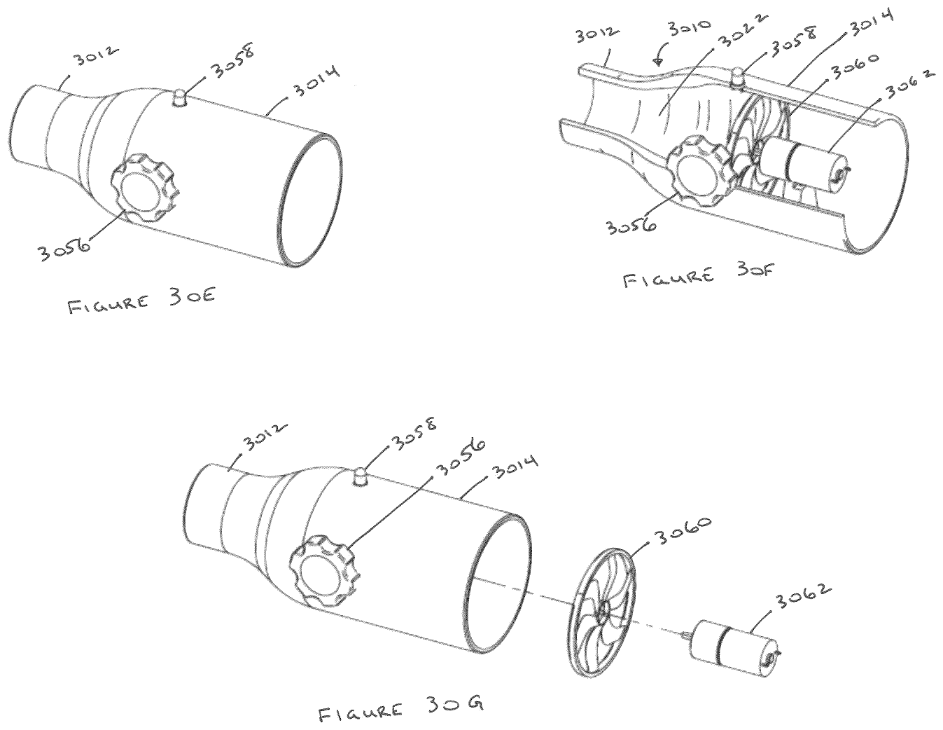

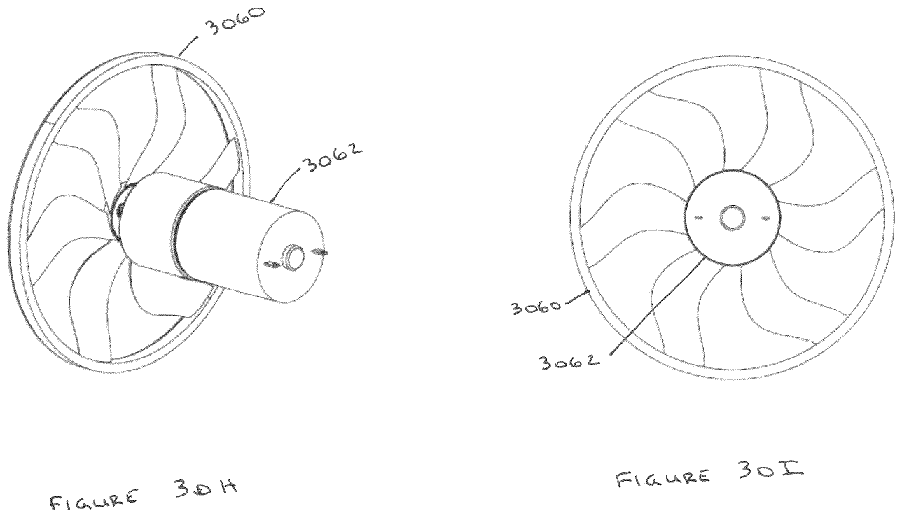

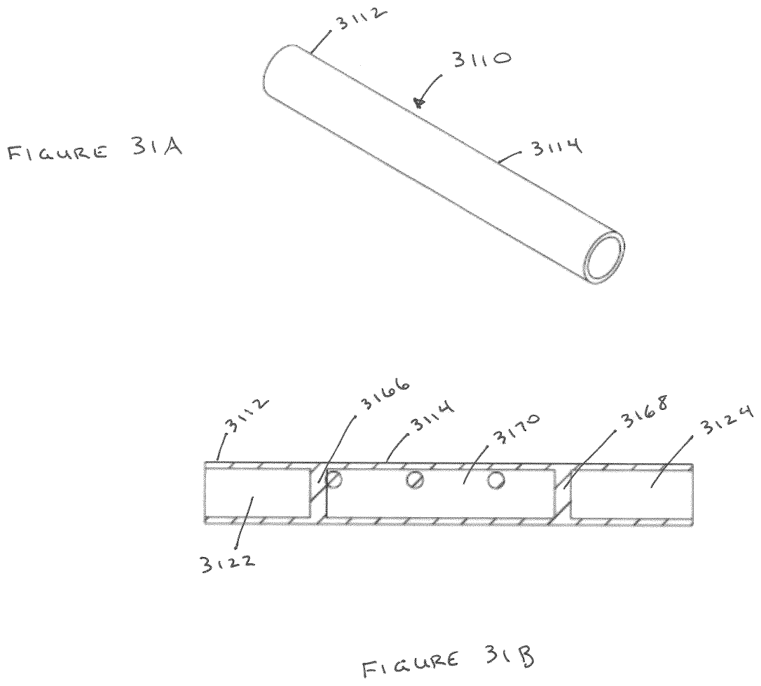

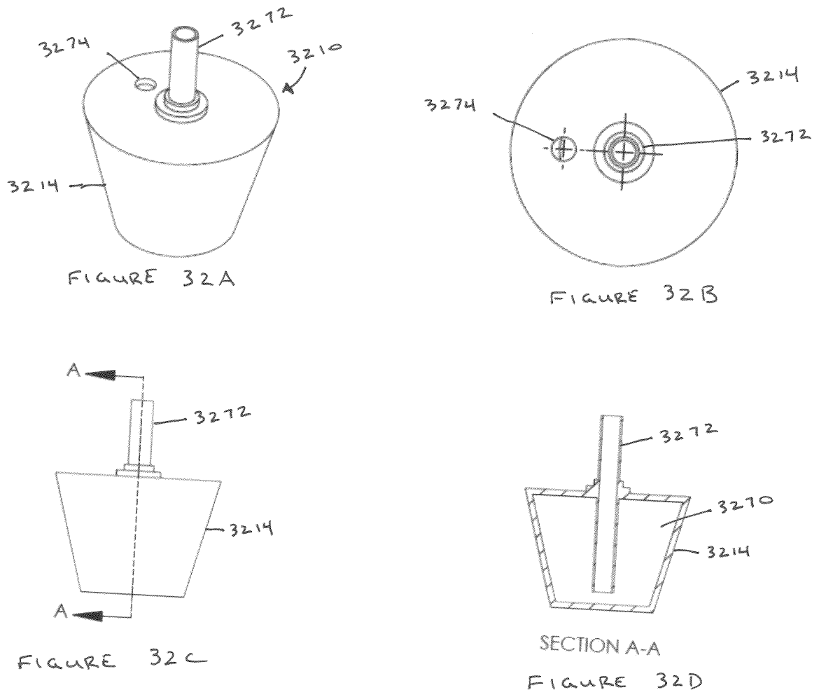

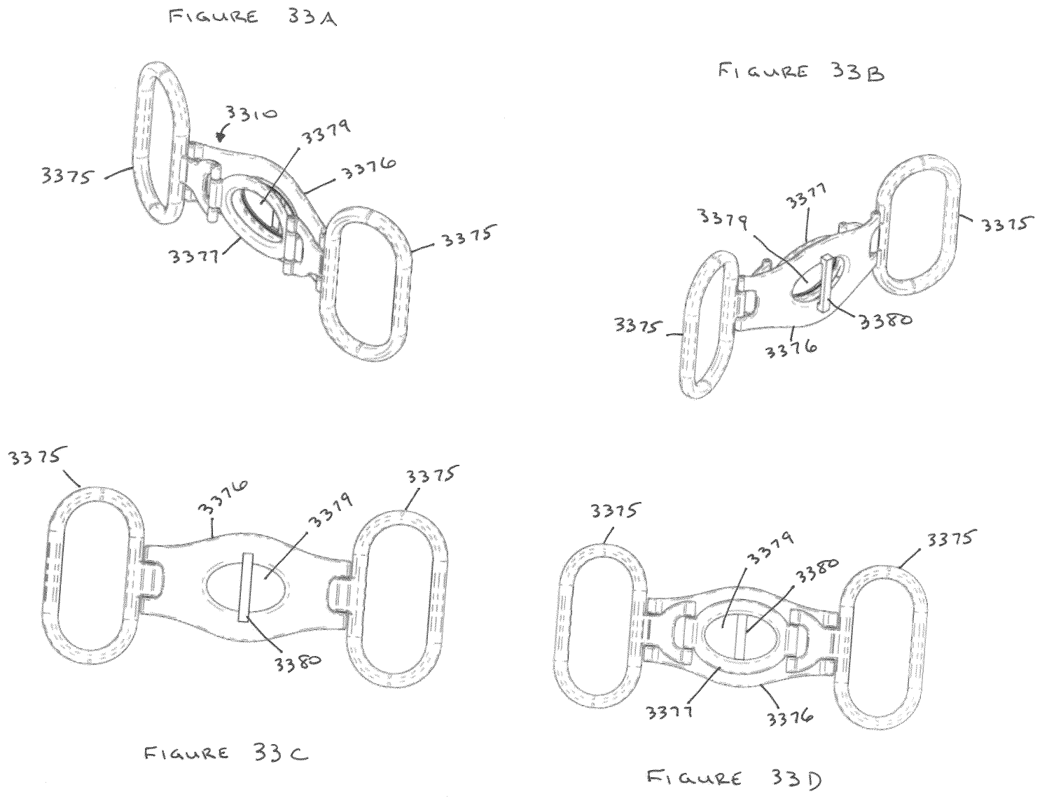

Referring to FIGS. 30A, 30B, 30C, 30D, 30E, 30F, 30G, 30H, and 30I, other embodiments of head and neck exercise apparatuses 3010 are shown. The head and neck exercise apparatuses 3010 may include a mask interface that connects to a member 3014 at the proximal interface 3012. The mask interface may be a nasal mask interface 3004 or an oronasal mask interface 3002. A member 3014 may define a passageway or proximal portion 3022 there through. The member 3014 may also define a passageway or distal portion 3024 there through. A rotating element 3060, motor 3062, resistance selector 3056, and/or airflow indicator 3058 may be operatively arranged with (e.g., disposed within, connected to, etc.) the member 3014. The resistance selector 3056 may be a variable resistor such as a potentiometer that cooperates with the motor 3062 in order to provide resistance to airflow through vents in the rotating element 3060; these collectively may be considered an airflow resistance mechanism.

The airflow indicator 3058 may be configured to respond with visual, auditory, or other feedback to airflow above a threshold rate, duration, or volume. One possible configuration would involve the motor 3062 serving as a generator to deliver electrical power to the airflow indicator 3058, but other configurations are possible. The rotating element 3060 may be configured to be adjustable and/or to respond to airflow in either direction. In this embodiment, the rotating element 3060 moves in a freely rotating fashion, but other movements (translation, limitation on rotation, etc.) are possible. The head and neck exercise apparatus 3014 may be configured to permit substitution of the rotating element 3060, motor 3062, resistance selector 3056, and/or airflow indicator 3058. The member 3014, rotating element 3060, motor 3062, resistance selector 3056, and/or airflow indicator 3058 may be configured to incorporate a valve mechanism that may alter resistance to airflow. A configuration to permit adjustment or substitution of at least one component may therefore result in a modifiable airflow resistance.

Referring to FIGS. 31A and 31B, another embodiment of a head and neck exercise apparatus 3110 is shown. The head and neck exercise apparatus 3110 may include a proximal portion 3112 and a member 3114. The member 3114 may define a passageway or proximal portion 3122 there through. A proximal semi-permeable membrane 3166 and distal semi-permeable membrane 3168 may be operatively arranged with (e.g., disposed within, connected to, etc.) the member 3114. The member 3114 may define a central chamber 3170 between the proximal semi-permeable membrane 3166 and distal semi-permeable membrane 3168. The member 3114 may also define a passageway or distal portion 3124 there through. The resistance to airflow through the head and neck exercise apparatus 3110 may be associated with the properties of the proximal semi-permeable membrane 3166, distal semi-permeable membrane 3168, and any substances within the central chamber 3170. The central chamber 3170 may contain gas, liquid and/or solid material, with properties (e.g., liquid viscosity, etc.) that may be associated with airflow resistance through the head and neck exercise apparatus 3110. Collectively, the proximal semi-permeable membrane 3166, distal semi-permeable membrane 3168, and central chamber 3170 may be considered an airflow resistance mechanism.

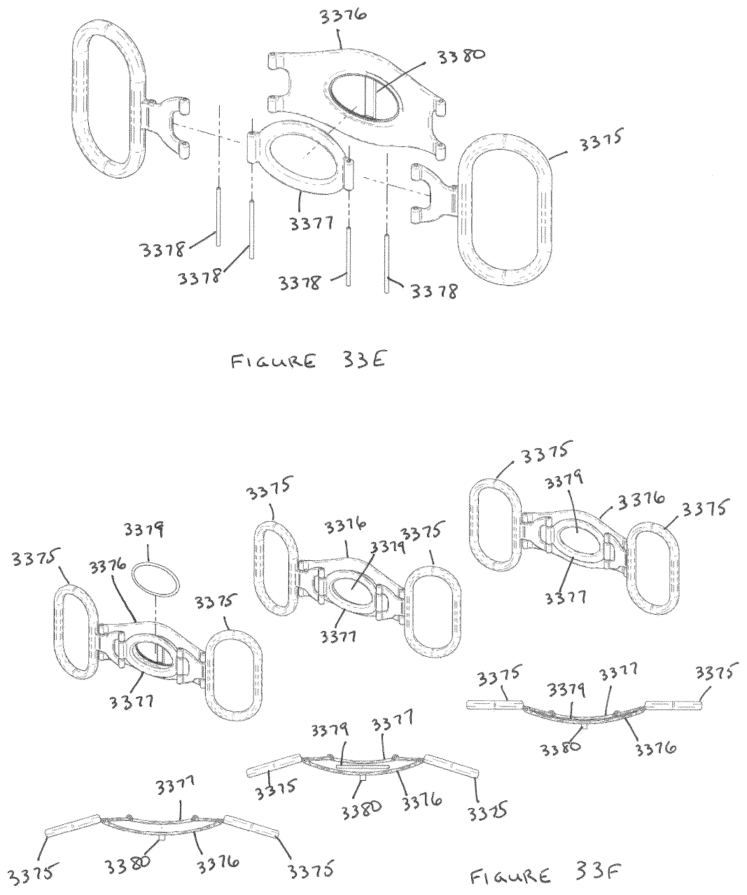

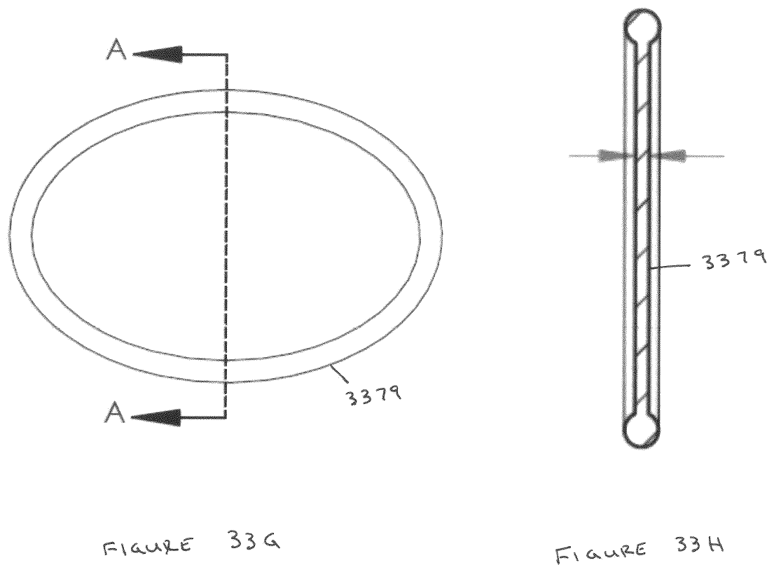

Referring to FIGS. 32A, 32B, 32C, and 32D, another embodiment of a head and neck exercise apparatus 3210 is shown. The head and neck exercise apparatus 3210 may include a proximal interface element 3272 and a member 3214. The member 3214 may define a central chamber 3270 within it. The proximal interface element 3272 may be inserted to variable depths within the central chamber 3270. The proximal interface element 3272 may include at least one valve and/or semi-permeable membrane. The vent 3274 may include at least one valve mechanism and/or semi-permeable membrane. The central chamber 3270 may contain gas, liquid, and/or solid material, with differential properties (e.g., density, liquid viscosity). The resistance to airflow through the head and neck exercise apparatus 3210 may be associated with the depth of insertion of the proximal interface element 3272, properties of material within the central chamber 3270, or the properties of the proximal interface element 3272 or vent 3274. Collectively, the proximal interface element 3272, central chamber 3270, and vent 3274 may be considered an airflow resistance mechanism.

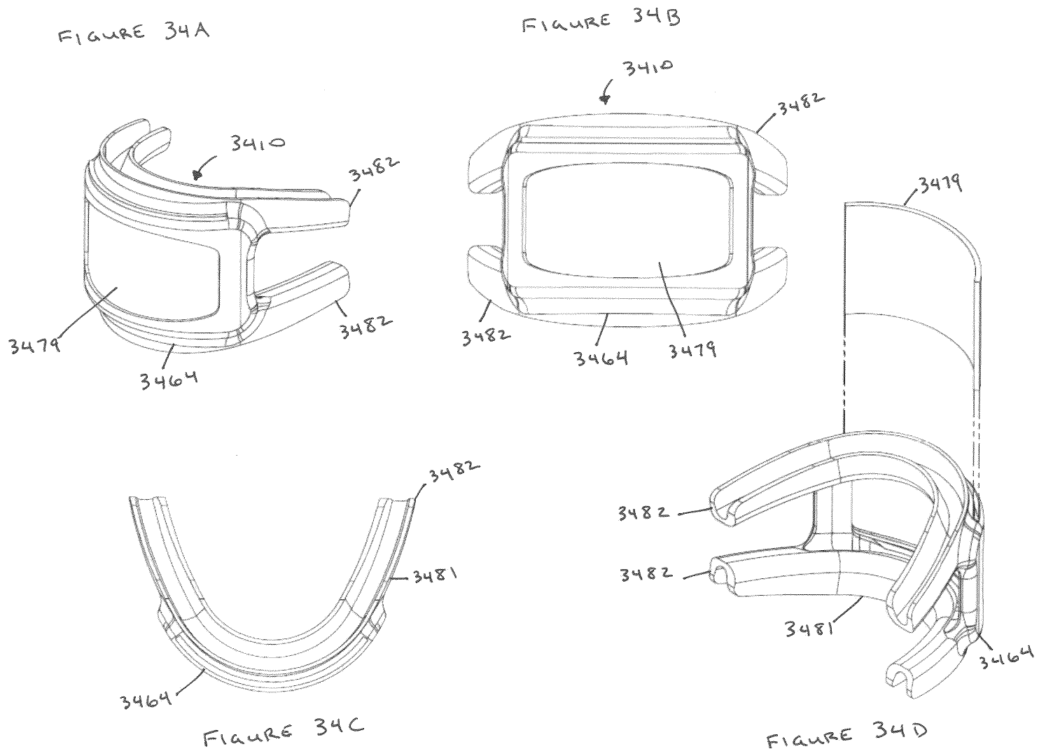

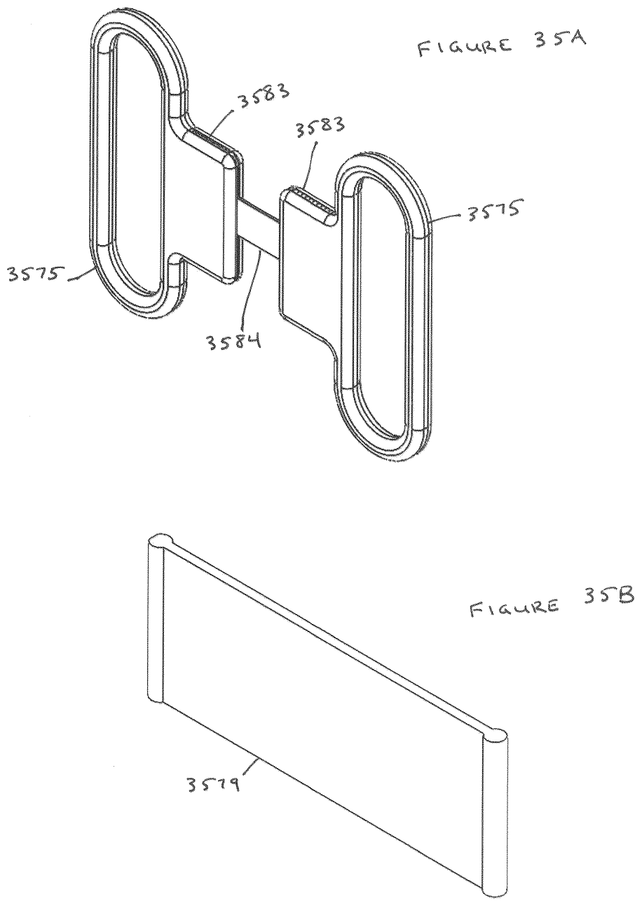

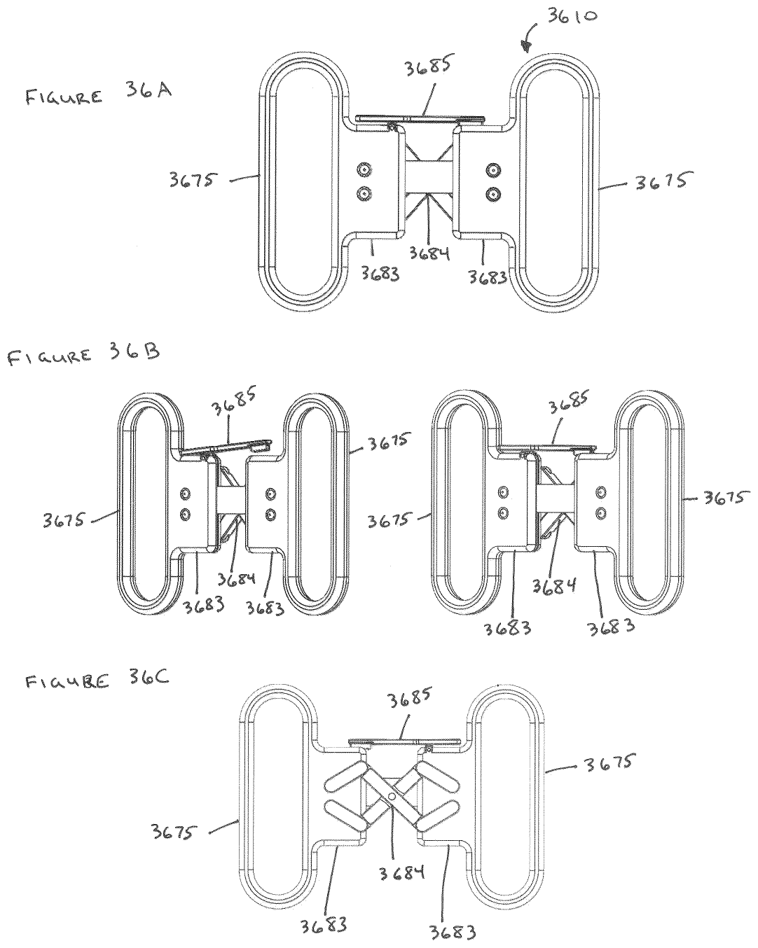

Referring to FIGS. 33A, 33B, 33C, 33D, 33E, 33F, 33G, and 33H, another embodiment of a head and neck exercise apparatus 3310 is shown. The head and neck exercise apparatus 3310 may include outer pieces 3375 that enable a user to position the head and neck exercise apparatus 3310 against a portion of their head and neck. A front housing piece 3376 and back housing piece 3377 hold a moveable element 3379 in place, such that the user may protrude a portion of their head and neck against the moveable element 3379. In this embodiment, the user may protrude a portion of their head and neck to displace the moveable element 3379 to a distance determined by the position of the contact element 3380. The user may be able to sense (by sight, touch, etc.) the contact element 3380 as they displace the moveable element 3379 to contact the contact element 3380. The contact element 3380 or any other component of the head and neck exercise apparatus 3310 may be configured to provide auditory, vibratory, or other feedback during performance of the head and neck exercise. In this embodiment, the user may protrude their tongue against the moveable element 3379, but in other embodiments the user may displace the moveable element using other portions of the head and neck (mandible, etc.).

In this embodiment, the head and neck exercise apparatus 3310 includes hinge pins 3378 that enable relative movement of the front housing piece 3376 and back housing piece 3377 as the outer pieces 3375 move back and forth. In this embodiment, the space between the front housing piece 3376 and back housing piece 3377 opens to permit substitution of the moveable element 3379, and movement of the outer pieces 3375 may close the space to secure the moveable element 3379 in place. The moveable element 3379 may be secured in position by the cooperation between the moveable element 3379, front housing piece 3376, and back housing piece 3377. In this embodiment, the periphery of the moveable element 3379 is thicker than the central portion to facilitate this cooperation, but other configurations (periphery of similar or lesser thickness, differences in shape, etc.) or mechanisms for cooperation (e.g., elements extending through the moveable element, etc.) are possible. The head and neck exercise apparatus 3310 may be configured with a mechanism to maintain the outer pieces 3375 in at least one position, for example, in a position with a closed space. The substitution of a moveable element 3379 with another moveable element 3379 may be considered a different head and neck exercise apparatus.

In this embodiment, the moveable element 3379 is composed of an oval-shaped flexible material (e.g., a membrane), but the moveable element 3379 may take other forms, including a flexible material of different shapes or sizes or any other configuration that permits movement (e.g., flexible material with some relatively rigid segments, lever arm, moveable component, etc.).

The resistance to displacement of the moveable element 3379 may be determined by properties of the moveable element 3379 (e.g., stiffness, thickness, size, etc.) or other components of the head and neck exercise apparatus 3310. The head and neck exercise apparatus 3310 may also include a mechanism (e.g., spring, lever, etc.) to adjust the resistance of a single moveable element 3379. A substitution or adjustment of any component of the head and neck exercise apparatus 3310 may be considered a modification, and any such modification may modify the resistance to displacement of the moveable element 3379.

The contact element 3380 may be manufactured in continuity with the front moveable element holder piece 3376, or they may be separable. The moveable element 3379 may be manufactured in continuity with the front housing piece 3376 and/or back housing piece 3377. In other embodiments, the outer pieces 3375 may have a different configuration that enables the user to position the head and neck exercise apparatus 3310 against a portion of the user's head and neck such that it remains in proper position without active assistance from the user.

The outer pieces 3375, front housing piece 3376, back housing piece 3377, hinge pins 2078, and contact element 3380 may be considered a housing, within which there may be additional components of the head and neck exercise apparatus 3310 or at least one component of at least one other head and neck exercise apparatus.

In this embodiment, the user may protrude their tongue in the performance of the head and neck exercise. In other embodiments, the head and neck exercise may involve other tongue movements (retrusion, lateral movements, etc.); such an exercise may require the modification or addition of components (e.g., clamps, moveable elements, etc.) to the head and neck exercise apparatus 2010.

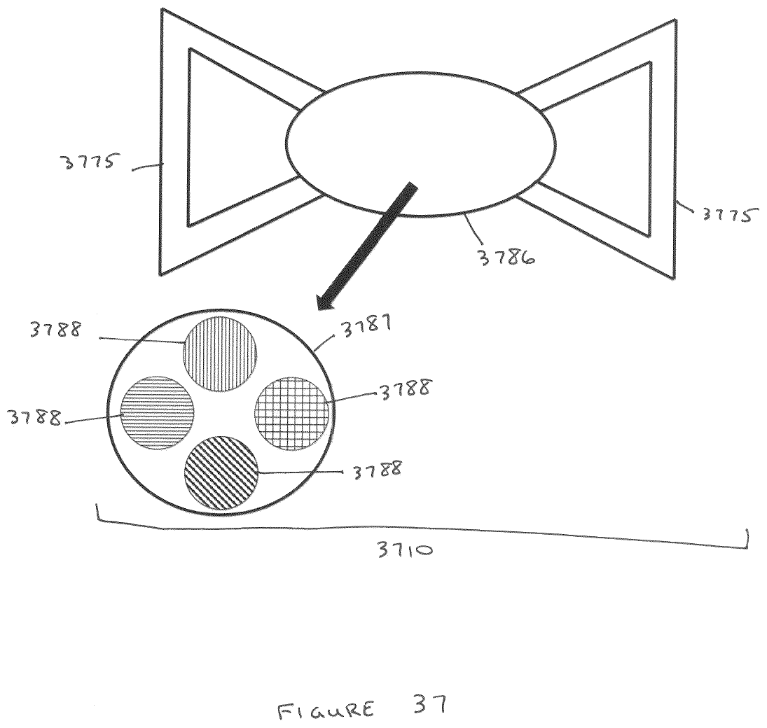

Referring to FIGS. 34A, 34B, 34C, and 34D, another embodiment of a head and neck exercise apparatus 3410 is shown. The head and neck exercise apparatus 3410 may include a mouthpiece 3481. The mouthpiece 3481 may include a moveable element holder frame 3464 that may be configured to secure a moveable element 3479. In this embodiment, the user may hold the mouthpiece 3481 in a stable position by placing their teeth into at least one dental tray 3482. The user may protrude their tongue against the moveable element 3479 to displace it anteriorly. The resistance to displacement of the moveable element 3479 may be determined by properties of the moveable element 3479 (e.g., stiffness, thickness, size, etc.) or other components of the head and neck exercise apparatus 3410. The head and neck exercise apparatus 3410 may include a mechanism to secure the moveable element 3479 in the moveable element holder frame 3464. The head and neck exercise apparatus 3410 may be configured without dental trays 3482, as the head and neck exercise apparatus 3410 may be used inside or outside the user's mouth. In another embodiment, the moveable element 3479 may be placed against a portion of the head and neck without a mouthpiece 3481. In other embodiments, the moveable element 3479 may be placed inside the mouth without a mouthpiece 3481.

In another embodiment, it may be possible to perform head and neck exercises with the moveable element 3479 but with fewer components of the head and neck exercise apparatus, including no other components.

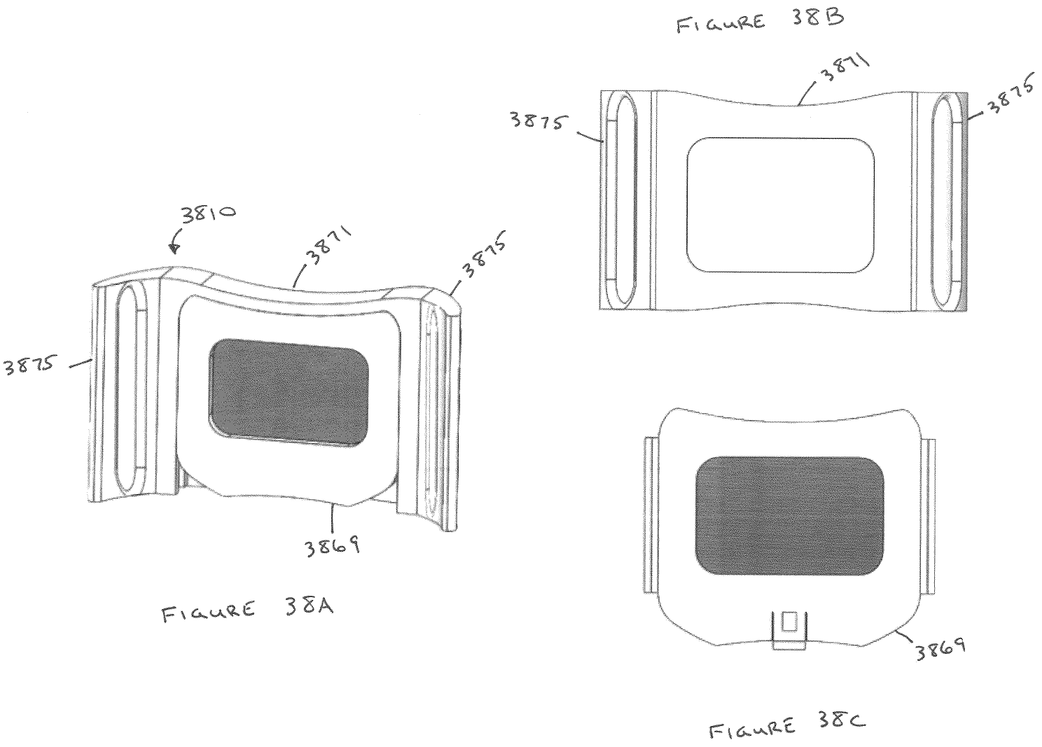

Referring to FIGS. 35A and 35B, another embodiment of a head and neck exercise apparatus is shown. The head and neck exercise apparatus may include outer pieces 3575 connected to moveable element holders 3583. There may be a stabilizing mechanism 3584 to limit the relative motion of the outer pieces 3575 and/or moveable element holders 3583. The moveable element holders 3583 may hold a moveable element 3579 on either side. The user may position the head and neck exercise apparatus against a portion of the head and neck, allowing them to protrude a portion of their head and neck to displace the moveable element 3579. In this embodiment, the user may protrude their tongue against the moveable element 3579, but in other embodiments the user may displace the moveable element 3579 using other portions of the head and neck (mandible, etc.).

The resistance to displacement of the moveable element 3579 may be determined by properties of the moveable element 3579 (e.g., stiffness, thickness, size, etc.) or other components of the head and neck exercise apparatus. The resistance to displacement of the moveable element 3579 may also be determined by any stretching of the moveable element 3579, for example by increasing the distance between the moveable element holders 3583. An adjustment or substitution of any component of the head and neck exercise apparatus may be considered a modification. In this embodiment, the stabilizing mechanism 3584 is straight, but in other embodiments it could incorporate at least one hinged and/or moveable component. In this embodiment, the outer pieces 3575 are in continuity with the moveable element holders 3583, but they may be separable in other embodiments.

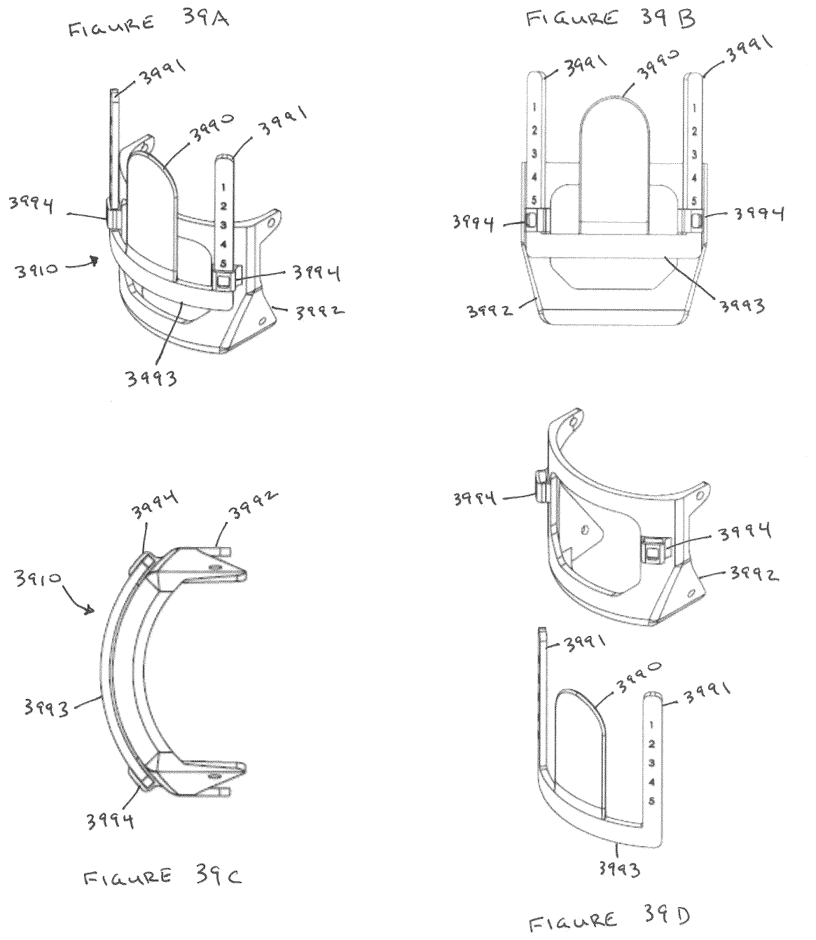

Referring to FIGS. 36A, 36B, and 36C, another embodiment of a head and neck exercise apparatus 3610 in shown. The head and neck exercise apparatus 3610 may include outer pieces 3675 connected to moveable element holders 3683. The stabilizing mechanism 3684 may connect the handle 3675 or moveable element holders 3683. The stabilizing mechanism 3684 may include fixed and moveable elements, as in this embodiment. There may be a lateral securing mechanism 3685 to provide additional stability of the relative position of the moveable element holders 3683 and/or outer pieces 3675.

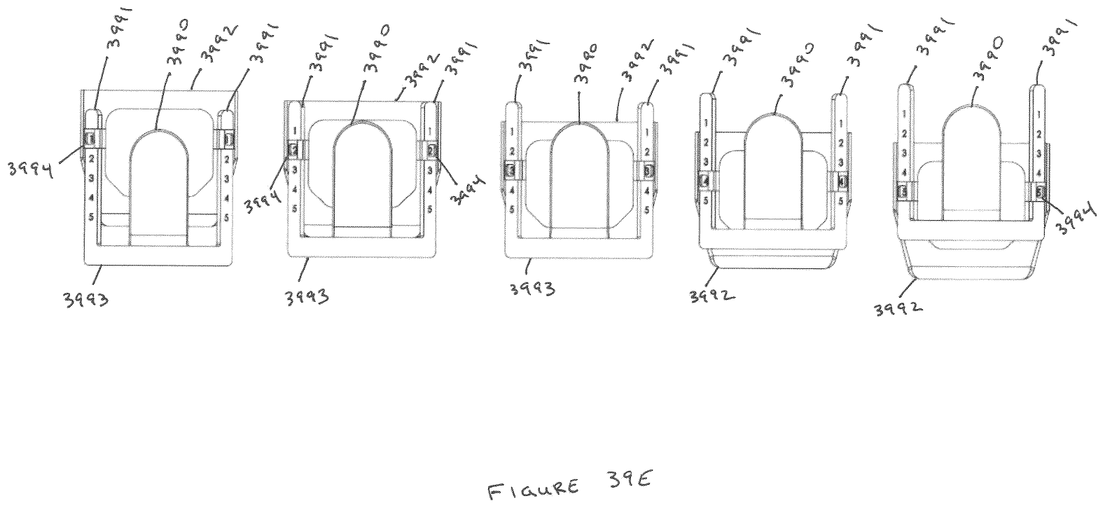

Referring to FIG. 37, another embodiment of a head and neck exercise apparatus 3710 is shown. The head and neck exercise apparatus may include outer pieces 3775 and a moveable element casing 3786. The moveable element casing 3786 may be configured to secure a moveable element selector 3787 that contains at least two moveable element sections 3788. The resistance to displacement of a moveable element section 3788 may be determined by properties of the moveable element section 3788 (e.g., stiffness, thickness, size, etc.) or other components of the head and neck exercise apparatus 3710. The at least two moveable element sections 3788 may be distinguished by different resistances to displacement. For the performance of a head and neck exercise, a user could then position the moveable element section 3788 corresponding to the desired level of resistance to displacement. The moveable element selector 3787 and/or moveable element sections 3788 could be substituted to enable a greater number of potential resistances to displacement. In this embodiment, the outer pieces 3775 are in continuity with the moveable element casing 3786, but they may be separable in other embodiments.

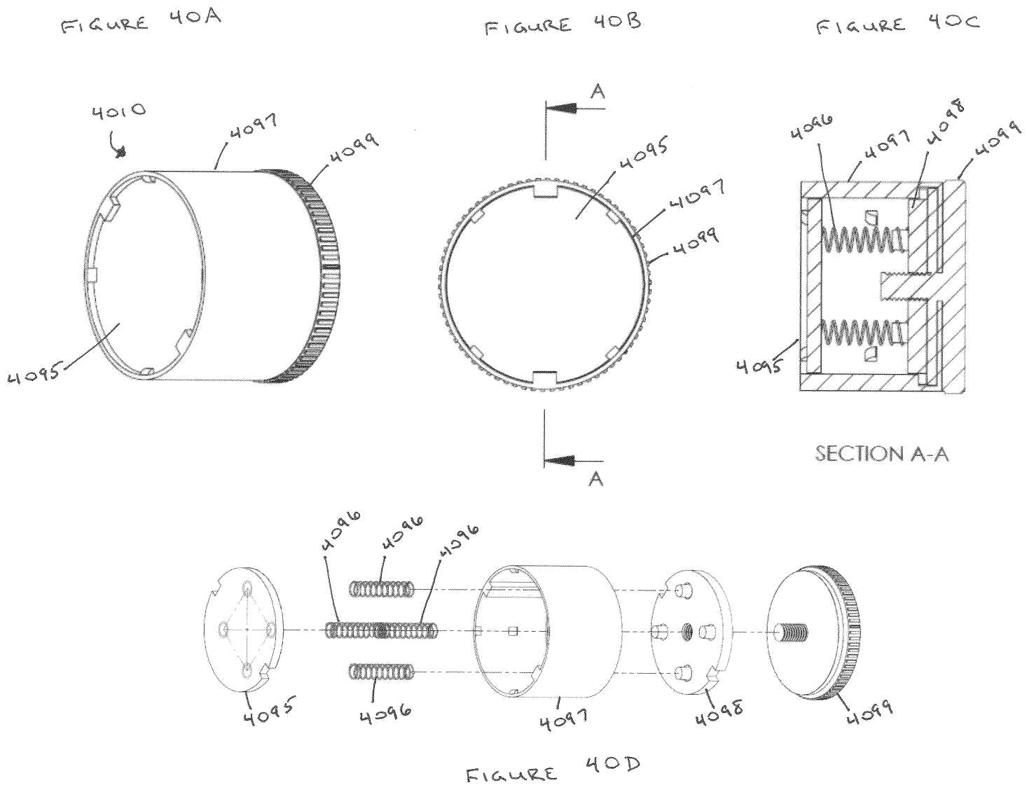

Referring to FIGS. 38A, 38B, and 38C, another embodiment of a head and neck exercise apparatus 3810 is shown. The head and neck exercise apparatus 3810 may include a framed moveable element 3869, a frame dock 3871, and at least one outer piece 3875. The framed moveable element 3869 may be configured to cooperate reversibly or irreversibly with the frame dock, with the same possible mechanisms of cooperation and advantages of reversible cooperation described elsewhere in this application. The framed moveable element 3869 may have similarities with moveable elements described elsewhere in this application. For example, the resistance to displacement of the framed moveable element 3869 may be determined by properties of the moveable element 3869 (e.g., stiffness, thickness, size, etc.) or other components of the head and neck exercise apparatus 3810.

The framed moveable element 3869 may have certain advantages, including simpler positioning of a moveable component and improving performance of the head and neck exercise apparatus. There may also be disadvantages, including increased cost.

In this embodiment the outer pieces 3875 and frame dock 3871 are manufactured in continuity, but in other embodiments they may be separable.

Referring to FIGS. 39A, 39B, 39C, 39D, and 39E, another embodiment of a head and neck exercise apparatus 3910 is shown. The head and neck exercise apparatus 3910 may include a frame piece 3992 and a resistance piece 3993. The frame piece 3992 may include at least one resistance indicator 3994. The resistance piece 3993 may include a sliding lever 3990 and resistance markers 3991. The frame piece 3992 may cooperate with the resistance piece 3993 at the at least one resistance indicator 3994. A user may position the head and neck exercise apparatus 3910 to a portion of the user's head and neck. In this embodiment, the user protrudes their tongue against the sliding lever 3990 at a position on the sliding lever 3990 corresponding to the two resistance indicators 3994. Changes in the relative position of the frame piece 3992 and resistance piece 3993 would change the relative position of the resistance indicators 3994 and may be associated with changes in the resistance to displacement of the sliding lever 3990 because the user would apply the displacement force against different positions on the sliding lever 3990. A displacement element may be added to indicate the desired distance of displacement during the performance of the head and neck exercise.

Referring to FIGS. 40A, 40B, 40C, and 40D, another embodiment of a head and neck exercise apparatus 4010 is shown. The head and neck exercise apparatus 4010 may include a moveable element 4095, at least one spring element 4096, case 4097, spring base 4098, and base 4099. The at least one spring element 4096 cooperates with the moveable element 4095 and spring base 4098. Resistance to displacement of the moveable element 4095 may be associated with the relative position of the moveable element 4095 and spring base 4098 as well as properties of the at least one spring element 4096 (e.g., number, spring constant, etc.), moveable element 4095 (e.g., size, weight, etc.), or any other component of the head and neck exercise apparatus 4010. In this embodiment, adjustment of the base 4099 may change the relative position of the moveable element 4095 and spring base 4098, but other configurations are possible. In this embodiment, there are four spring elements 4096, but other configurations are possible. The head and neck exercise apparatus 4010 may be configured to include features or components that assist in positioning against a portion of the head and neck.

Head and neck exercises for sleep disordered breathing may include isotonic and/or isometric exercises that can involve muscles within and surrounding the oral cavity and/or pharynx. Methods have been proposed previously for head and neck exercises. These have generally not included the use of an apparatus or system. Some methods for head and neck exercises for sleep disordered breathing have included the use of apparatuses to brush or stroke the tongue or a single positive end-expiratory pressure valve with an adjustable threshold pressure, as described above.

Instead, printed, video, and/or audio materials may instruct the user in performance of a head and neck exercise for sleep disordered breathing using the apparatuses or system described elsewhere in this application. These materials may be pre-recorded or live and can be uniform or tailored to an individual or subgroup. After placement of the apparatus or system to the user's mouth, exercises may include the generation of positive or negative pressure within the head and neck apparatus or system. A user may be instructed to generate positive or negative pressure within the apparatus or system by activation of muscles of the oral cavity and/or pharynx. A user may also be instructed to generate positive or negative pressure within the apparatus or system by exhalation or inspiration. A user may also be instructed to protrude a portion of their head and neck against an apparatus or system by activation of muscles of the oral cavity or pharynx. The instructions may present the technique for performance of the exercise, including possible modifications. The method of instructing the user may include any aspect of exercise performance such as repetitions, duration of time, use of feedback information to indicate proper performance, or other parameters.

The method of instructing the user may match the features of any apparatus or system described elsewhere in this application. For example, a method may instruct a user to perform a head and neck exercise for sleep disordered breathing while positioning a head and neck exercise apparatus to the user's nose and creating a positive pressure within the proximal portion greater than a threshold pressure that is adjustable or not adjustable. The positive pressure may cause a valve mechanism operatively arranged with the member and biased towards a closed position to open when the positive pressure exceeds the threshold pressure such that the proximal portion is fluidly connected to a distal portion. The method of instructing the user may indicate the response that should occur with proper performance of the exercise. The threshold pressure may be based on at least one of a stiffness of the valve mechanism, a dimension of the valve mechanism, a dimension of the valve plate, or a dimension of the member.

In another embodiment, a method may instruct a user to perform a head and neck exercise for sleep disordered breathing while positioning a head and neck exercise apparatus to the user's nose and creating a negative pressure within the proximal portion greater than a threshold pressure that is adjustable or not adjustable. The negative pressure may cause a valve mechanism operatively arranged with the member and biased towards a closed position to open when the negative pressure is greater than the threshold pressure such that the proximal portion is fluidly connected to a distal portion. The method of instructing the user may indicate the response that should occur with proper performance of the exercise. The threshold pressure may be based on at least one of a stiffness of the valve mechanism, a dimension of the valve mechanism, a dimension of the valve plate, or a dimension of the member.

In another embodiment, a method may instruct a user to perform a head and neck exercise for sleep disordered breathing while positioning a head and neck exercise apparatus to the user's nose and creating a positive pressure within the proximal portion greater than a threshold pressure. The positive pressure may cause a valve mechanism operatively arranged with the member and biased towards an open position to close when the positive pressure exceeds the threshold pressure such that this blocks the fluid connection between the proximal portion and a distal portion that exists when the valve mechanism is in the open position. The threshold pressure may be adjustable or not adjustable. The apparatus may include at least one of an acoustic device, membrane, chromogenic material, or moveable element, each operatively arranged with the member and configured to respond to a pressure in the proximal portion being greater in magnitude than the threshold pressure.

In another embodiment, a method may instruct a user to perform a head and neck exercise for sleep disordered breathing while positioning a head and neck exercise apparatus to the user's nose and creating a negative pressure within the proximal portion greater than a threshold pressure. The negative pressure may cause a valve mechanism operatively arranged with the member and biased towards an open position to close when the negative pressure is greater than the threshold negative pressure such that this blocks the fluid connection between the proximal portion and a distal portion that exists when the valve mechanism is in the open position. The threshold pressure may be adjustable or not adjustable.

In another embodiment, a method may instruct a user to perform a head and neck exercise for sleep disordered breathing while positioning a head and neck exercise apparatus to the user's nose and creating a positive pressure within the proximal portion to produce an airflow or airflow rate against an airflow resistance determined by an airflow resistance mechanism that may be operatively arranged with a member. The apparatus may include at least one airflow resistance mechanism. The apparatus may or may not include an adjustment mechanism configured to alter the airflow resistance mechanism. The method of instructing the user may indicate the response that should occur with proper performance of the exercise, including production of a sufficient airflow or airflow rate. The apparatus may include at least one of an acoustic device, membrane, chromogenic material, visual signal, or moveable element, each operatively arranged with the member and configured to respond to an airflow or airflow rate greater in magnitude than a threshold airflow or threshold airflow rate, respectively. The airflow rate through the member may be based on at least one of a dimension of the member or a characteristic of the airflow resistance mechanism. A breathing exercise system may comprise a combination of head and neck exercise apparatuses.

In another embodiment, a method may instruct a user to perform head and neck exercises for sleep disordered breathing while positioning a head and neck exercise apparatus to the user and moving the tongue to displace a portion of a moveable element of the apparatus such that the portion of the moveable element is in contact with a component of the apparatus.

One characteristic of the apparatuses and systems described in this application is the threshold pressure or airflow resistance. A user may be instructed to perform Exercise 41 using Apparatus 41A having a first threshold pressure or airflow resistance at block 4151. Once the user is able to perform the exercise and meet certain criteria at block 4153 (e.g., number of repetitions, duration, or other means of determining a level of function), the user may be instructed to perform Exercise 41 with Apparatus 41B at block 4155, distinguished from Apparatus 41A by having a different threshold pressure or airflow resistance. Apparatus 41B may be a different physical device or have a different configuration from Apparatus 41A (e.g., with and without valve mechanism, different valve mechanism with different properties, different airflow resistance mechanism with different properties, etc.). Once the user meets certain criteria in the performance of Exercise 41 using Apparatus 41B at block 4157, the user may be instructed to perform Exercise 41 with Apparatus 41C at block 4159, with or without progression to other apparatuses. All permutations described elsewhere in this application are possible. Apparatus 41A (and any apparatus mentioned below) may correspond to any of the apparatus or head and neck exercise system configurations presented elsewhere in this application.

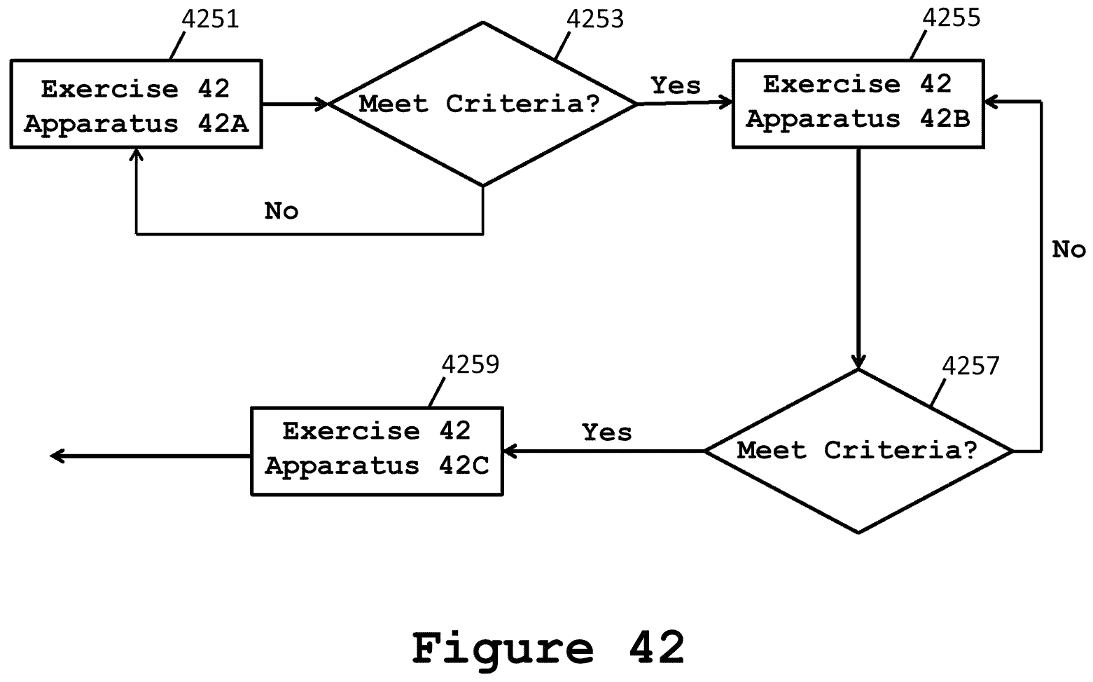

Referring to FIG. 42, a user may be instructed to perform Exercise 42 using Apparatus 42A having a first threshold pressure or airflow resistance at block 4251. Once the user is able to perform the exercise and meet certain criteria at block 4253 (e.g., number of repetitions, duration, or other means of determining a level of function), the user may be instructed to perform Exercise 42 with Apparatus 42B at block 4255, distinguished from Apparatus 42A by including a modification of Apparatus 42A. Once the user meets certain criteria in the performance of Exercise 42 using Apparatus 42B at block 4257, the user may be instructed to perform Exercise 42 with Apparatus 42C at block 4259, with or without progression to other apparatuses. All permutations described elsewhere in this application are possible.

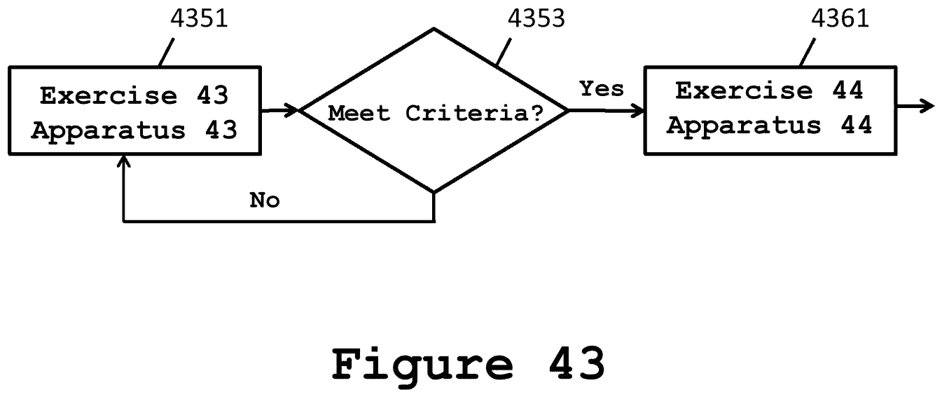

Referring to FIG. 43, a user may be instructed to perform Exercise 43 with Apparatus 43 at block 4351. Once the user meets criteria at block 4353, the user may be instructed to perform Exercise 44 using Apparatus 44 at block 4361. Apparatus 44 may be distinguished from Apparatus 43 by having a different threshold pressure, different airflow resistance mechanism, or different configuration from Apparatus 43. The user may be instructed to modify the performance of Exercise 43, Exercise 44, or another exercise using an apparatus according to whether they meet certain criteria (not shown), as described elsewhere in this application. In another embodiment, once the user meets criteria, the user may be instructed to perform the same or a different exercise without using any apparatus or system.

The user may be instructed to perform multiple head and neck exercises for sleep disordered breathing with the use of the same apparatus or different apparatuses. The user may also be instructed to perform head and neck exercises for sleep disordered breathing without the use of any apparatus or system.

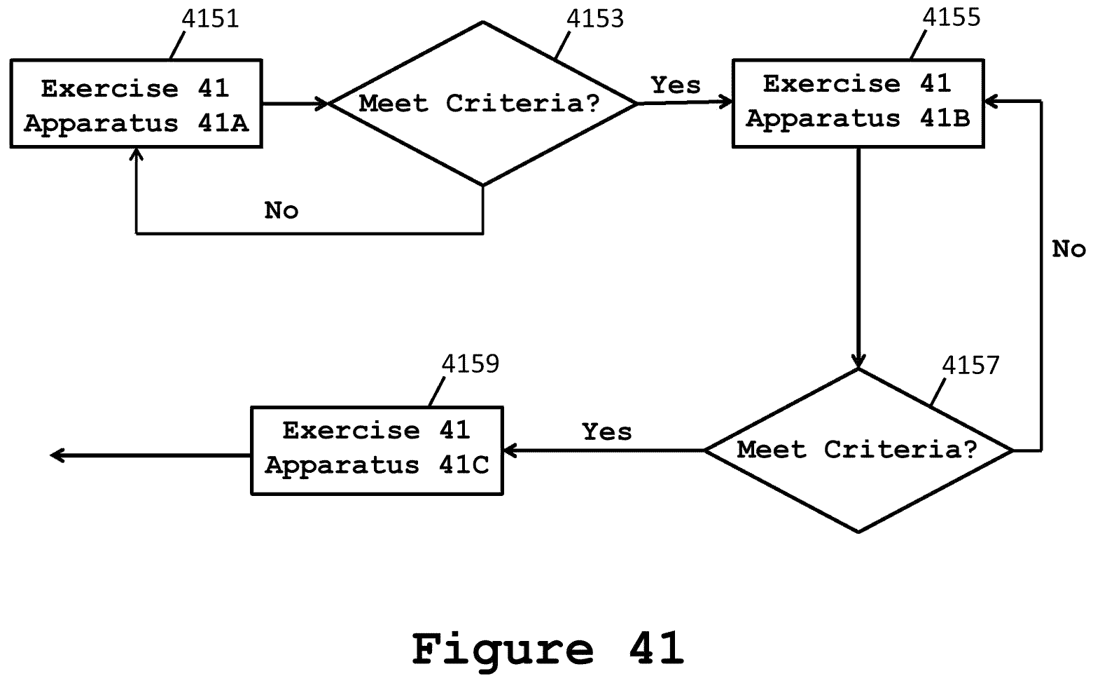

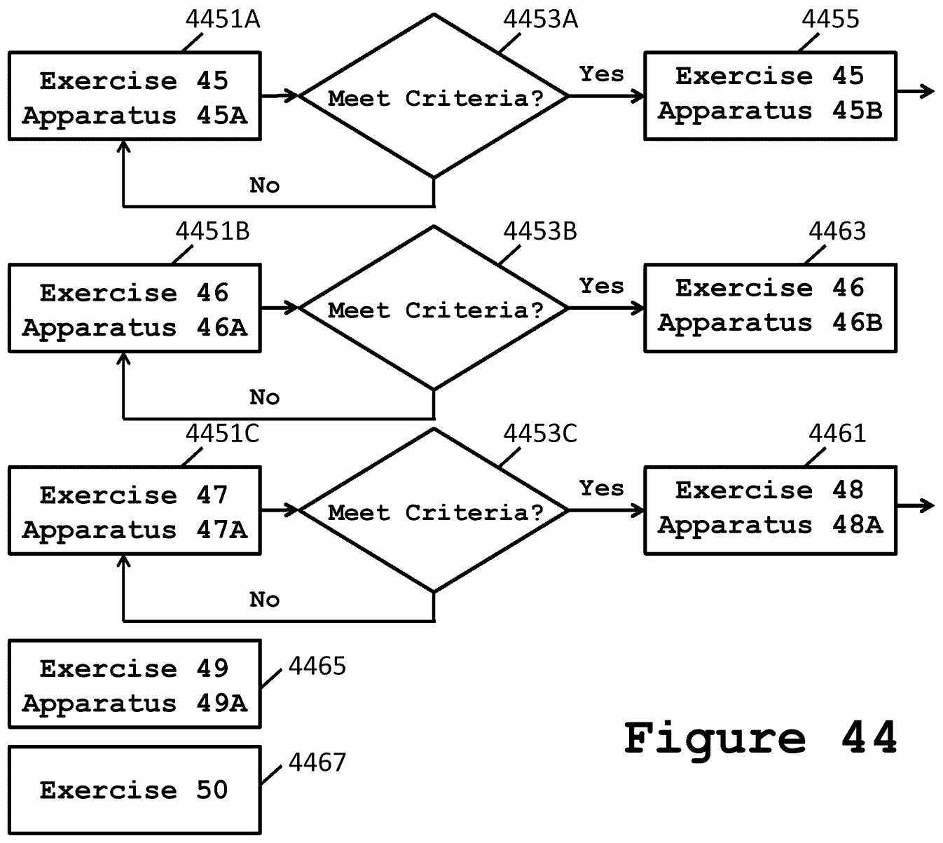

Referring to FIG. 44, a user may be instructed to perform at least one head and neck exercise for sleep disordered breathing using at least one head and neck exercise apparatus or system. The apparatuses or systems may incorporate different configurations and/or different threshold pressures. If the user performs at least two exercises, the exercises may be designed to achieve a goal when performed together. For example, the user may be instructed to perform Exercise 45 with Apparatus 45A at block 4451A, progressing if they meet criteria at block 4453A to using Apparatus 45B at block 4455 and, possibly, Apparatus 45C (not shown), similar to FIGS. 41 and 42. In a similar initial pattern, the user may be instructed to perform Exercise 46 with Apparatus 46A at block 4451B, with possible progression if they meet criteria at block 4453B to using Apparatus 46B at block 4463 but without additional progression to a different apparatus. In a similar initial pattern, the user may be instructed to perform Exercise 47 with Apparatus 47A at block 4451C, with progression if they meet criteria at block 4453C to Exercise 48 and Apparatus 48A at block 4461 once the user meets certain criteria. The user may be instructed to perform Exercise 49 with Apparatus 49A at block 4465, without progression to another apparatus. The user may be instructed to perform Exercise 50 without the use of an apparatus at block 4467. The user may be instructed to perform any permutation of head and neck exercises using these or other pathways. All permutations described elsewhere in this application are possible.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention. Moreover, certain embodiments may be used in head and neck exercises for other conditions or user concerns involving muscles and/or other tissues of the head and neck. Other conditions or user concerns can include disorders of speech or swallowing, skin wrinkles, fat in the head and neck, teeth grinding, facial pain, and mouth breathing.

What is claimed is:

1. A method for performing a breathing exercise with a breathing apparatus, the method comprising:
positioning an open portion of a member, of the apparatus, to a nose of a user such that breathing by the user through the open portion results in anterior movement of a soft palate of the user to open space in an area of a throat of the user behind the soft palate to create a continuous air pathway from the nose to lungs of the user; and
performing by the user the breathing exercise via the continuous air pathway, with the apparatus, in which the user generates, for a predetermined number of repetitions or duration of time, a pressure within the open portion that exceeds a threshold pressure of a valve mechanism, of the apparatus, biased towards a closed position to cause the valve mechanism to open such that the open portion of the apparatus is fluidly connected to a distal portion of the apparatus and the lungs to train muscles of the user.

2. A method for performing a breathing exercise with a breathing apparatus, the method comprising:
positioning an open portion of a member, of the apparatus, to a nose of a user such that breathing by the user through the open portion results in anterior movement of a soft palate of the user to open space in an area of a throat of the user behind the soft palate to create a continuous air pathway from the nose to lungs of the user; and
performing by the user the breathing exercise via the continuous air pathway, with the apparatus, in which the user generates, for a predetermined number of repetitions or duration of time, a pressure within the open portion that exceeds a threshold pressure of a valve mechanism, of the apparatus, biased towards an open position to cause the valve mechanism to close such that the open portion of the apparatus is fluidly connected to the lungs but separated from a distal portion of the apparatus to train muscles of the user.

3. The method of claim 1, further comprising repeating the performing by the user the breathing exercise with the breathing apparatus until predefined criteria are met;
responsive to the predefined criteria being met, positioning an open portion of a member, of another breathing apparatus, to a nose of a user such that breathing by the user through the open portion results in anterior movement of a soft palate of the user to open space in an area of a throat of the user behind the soft palate to create a continuous air pathway from the nose to lungs of the user;
performing by the user the breathing exercise via the continuous air pathway, with the another breathing apparatus, in which the user generates, for a predetermined or another predetermined number of repetitions or duration of time, a pressure within the open portion that exceeds a threshold pressure of a valve mechanism, of the another breathing apparatus, biased towards a closed position to cause the valve mechanism to open such that the open portion of the another breathing apparatus is fluidly connected to a distal portion of the another breathing apparatus and the lungs to train muscles of the user; and
repeating the performing by the user the breathing exercise with the another breathing apparatus.

4. The method of claim 1, further comprising repeating the performing by the user the breathing exercise with the breathing apparatus until predefined criteria are met;
responsive to the predefined criteria being met, positioning an open portion of a member, of another breathing apparatus, to a nose of a user such that breathing by the user through the open portion results in anterior movement of a soft palate of the user to open space in an area of a throat of the user behind the soft palate to create a continuous air pathway from the nose to lungs of the user;
performing by the user the breathing exercise via the continuous air pathway, with the another breathing apparatus, in which the user generates, for a predetermined or another predetermined number of repetitions or duration of time, a pressure within the open portion that exceeds a threshold pressure of a valve mechanism, of the another breathing apparatus, biased towards an open position to cause the valve mechanism to close such that the open portion of the another breathing apparatus is fluidly connected to the lungs but separated from a distal portion of the another breathing apparatus to train muscles of the user; and
repeating the performing by the user the breathing exercise with the another breathing apparatus.

5. The method of claim 2, further comprising repeating the performing by the user the breathing exercise with the breathing apparatus until predefined criteria are met;
responsive to the predefined criteria being met, positioning an open portion of a member, of another breathing apparatus, to a nose of a user such that breathing by the user through the open portion results in anterior movement of a soft palate of the user to open space in an area of a throat of the user behind the soft palate to create a continuous air pathway from the nose to lungs of the user;
performing by the user the breathing exercise via the continuous air pathway, with the another breathing apparatus, in which the user generates, for a predetermined or another predetermined number of repetitions or duration of time, a pressure within the open portion that exceeds a threshold pressure of a valve mechanism, of the another breathing apparatus, biased towards a closed position to cause the valve mechanism to open such that the open portion of the another breathing apparatus is fluidly connected to a distal portion of the another breathing apparatus and the lungs to train muscles of the user; and
repeating the performing by the user the breathing exercise with the another breathing apparatus.

6. The method of claim 2, further comprising repeating the performing by the user the breathing exercise with the breathing apparatus until predefined criteria are met;
responsive to the predefined criteria being met, positioning an open portion of a member, of another breathing apparatus, to a nose of a user such that breathing by the user through the open portion results in anterior movement of a soft palate of the user to open space in an area of a throat of the user behind the soft palate to create a continuous air pathway from the nose to lungs of the user;
performing by the user the breathing exercise via the continuous air pathway, with the another breathing apparatus, in which the user generates, for a predetermined or another predetermined number of repetitions or duration of time, a pressure within the open portion that exceeds a threshold pressure of a valve mechanism, of the another breathing apparatus, biased towards an open position to cause the valve mechanism to close such that the open portion of the another breathing apparatus is fluidly connected to the lungs but separated from a distal portion of the another breathing apparatus to train muscles of the user; and
repeating the performing by the user the breathing exercise with the another breathing apparatus.

7. The method of claim 1, wherein the valve mechanism of the breathing apparatus is modifiable to alter the threshold pressure.

8. The method of claim 2, wherein the valve mechanism of the breathing apparatus is modifiable to alter the threshold pressure.

9. The method of claim 3, wherein the valve mechanism of the another breathing apparatus is modifiable to alter the threshold pressure.

10. The method of claim 4, wherein the valve mechanism of the another breathing apparatus is modifiable to alter the threshold pressure.

11. The method of claim 5, wherein the valve mechanism of the another breathing apparatus is modifiable to alter the threshold pressure.

12. The method of claim 6, wherein the valve mechanism of the another breathing apparatus is modifiable to alter the threshold pressure.

13. The method of claim 1, further comprising selecting the breathing apparatus based on (i) an ability of the user to perform the breathing exercise, using the breathing apparatus with the threshold pressure, for a predetermined number of repetitions or duration of time, (ii) an ability of the user to perform the breathing exercise, using the breathing apparatus with a different threshold pressure, for the predetermined number of repetitions or duration of time, (iii) an ability of the user to perform the breathing exercise, using a different breathing apparatus with a different threshold pressure, for a different predetermined number of repetitions or duration of time, (iv) an ability of the user to activate head and neck muscles with a muscle strength or a muscle tension required to perform the breathing exercise using the breathing apparatus or the different breathing apparatus, or (v) the threshold pressure of the breathing apparatus.

14. The method of claim 2, further comprising selecting the breathing apparatus based on (i) an ability of the user to perform the breathing exercise, using the breathing apparatus with the threshold pressure, for a predetermined number of repetitions or duration of time, (ii) an ability of the user to perform the breathing exercise, using the breathing apparatus with a different threshold pressure, for the predetermined number of repetitions or duration of time, (iii) an ability of the user to perform the breathing exercise, using a different breathing apparatus with a different threshold pressure, for a different predetermined number of repetitions or duration of time, (iv) an ability of the user to activate head and neck muscles with a muscle strength or a muscle tension required to perform the breathing exercise using the breathing apparatus or the different breathing apparatus, or (v) the threshold pressure of the breathing apparatus.

15. The method of claim 3, further comprising selecting the another breathing apparatus based on (i) an ability of the user to perform the breathing exercise, using the breathing apparatus with the threshold pressure, for a predetermined number of repetitions or duration of time, (ii) an ability of the user to perform the breathing, using the breathing apparatus with a different threshold pressure, for the predetermined number of repetitions or duration of time, (iii) an ability of the user to perform the breathing exercise, using the another breathing apparatus with the another threshold pressure, for another predetermined number of repetitions or duration of time, (ii) an ability of the user to perform the breathing exercise, using the another breathing apparatus with a different threshold pressure, for the another predetermined number of repetitions or duration of time, (v) an ability of the user to perform the breathing exercise, using a different breathing apparatus with a different threshold pressure, for a different predetermined number of repetitions or duration of time, (vi) an ability of the user to activate head and neck muscles with a muscle strength or a muscle tension required to perform the breathing exercise using the breathing apparatus, the another head and neck exercise apparatus, or the different head and neck exercise apparatus, or (vii) the threshold pressure of the another breathing apparatus.

16. The method of claim 4, further comprising selecting the another breathing apparatus based on (i) an ability of the user to perform the breathing exercise, using the breathing apparatus with the threshold pressure, for a predetermined number of repetitions or duration of time, (ii) an ability of the user to perform the breathing, using the breathing apparatus with a different threshold pressure, for the predetermined number of repetitions or duration of time, (iii) an ability of the user to perform the breathing exercise, using the another breathing apparatus with the another threshold pressure, for another predetermined number of repetitions or duration of time, (ii) an ability of the user to perform the breathing exercise, using the another breathing apparatus with a different threshold pressure, for the another predetermined number of repetitions or duration of time, (v) an ability of the user to perform the breathing exercise, using a different breathing apparatus with a different threshold pressure, for a different predetermined number of repetitions or duration of time, (vi) an ability of the user to activate head and neck muscles with a muscle strength or a muscle tension required to perform the breathing exercise using the breathing apparatus, the another head and neck exercise apparatus, or the different head and neck exercise apparatus, or (vii) the threshold pressure of the another breathing apparatus.

17. The method of claim 5, further comprising selecting the another breathing apparatus based on (i) an ability of the user to perform the breathing exercise, using the breathing apparatus with the threshold pressure, for a predetermined number of repetitions or duration of time, (ii) an ability of the user to perform the breathing, using the breathing apparatus with a different threshold pressure, for the predetermined number of repetitions or duration of time, (iii) an ability of the user to perform the breathing exercise, using the another breathing apparatus with the another threshold pressure, for another predetermined number of repetitions or duration of time, (ii) an ability of the user to perform the breathing exercise, using the another breathing apparatus with a different threshold pressure, for the another predetermined number of repetitions or duration of time, (v) an ability of the user to perform the breathing exercise, using a different breathing apparatus with a different threshold pressure, for a different predetermined number of repetitions or duration of time, (vi) an ability of the user to activate head and neck muscles with a muscle strength or a muscle tension required to perform the breathing exercise using the breathing apparatus, the another head and neck exercise apparatus, or the different head and neck exercise apparatus, or (vii) the threshold pressure of the another breathing apparatus.

18. The method of claim 6, further comprising selecting the another breathing apparatus based on (i) an ability of the user to perform the breathing exercise, using the breathing apparatus with the threshold pressure, for a predetermined number of repetitions or duration of time, (ii) an ability of the user to perform the breathing, using the breathing apparatus with a different threshold pressure, for the predetermined number of repetitions or duration of time, (iii) an ability of the user to perform the breathing exercise, using the another breathing apparatus with the another threshold pressure, for another predetermined number of repetitions or duration of time, (ii) an ability of the user to perform the breathing exercise, using the another breathing apparatus with a different threshold pressure, for the another predetermined number of repetitions or duration of time, (v) an ability of the user to perform the breathing exercise, using a different breathing apparatus with a different threshold pressure, for a different predetermined number of repetitions or duration of time, (vi) an ability of the user to activate head and neck muscles with a muscle strength or a muscle tension required to perform the breathing exercise using the breathing apparatus, the another head and neck exercise apparatus, or the different head and neck exercise apparatus, or (vii) the threshold pressure of the another breathing apparatus.

19. The method of claim 1, further comprising repeating the performing by the user the breathing exercise with the breathing apparatus until predefined criteria are met; and responsive to the predefined criteria being met, modifying the threshold pressure of the valve mechanism.

20. The method of claim 2, further comprising repeating the performing by the user the breathing exercise with the breathing apparatus until predefined criteria are met; and responsive to the predefined criteria being met, modifying the threshold pressure of the valve mechanism.

21. The method of claim 3, further comprising repeating the performing by the user the breathing exercise with the another breathing apparatus until predefined criteria are met; and
responsive to the predefined criteria being met, modifying the threshold pressure of the valve mechanism.

22. The method of claim 6, further comprising repeating the performing by the user the breathing exercise with the another breathing apparatus until predefined criteria are met; and responsive to the predefined criteria being met, modifying the threshold pressure of the valve mechanism.

23. The method of claim 19, wherein the modifying the threshold pressure is based on (i) an ability of the user to perform the breathing exercise, using the breathing apparatus with the threshold pressure or another threshold pressure, for the predetermined number of repetitions or duration of time, (ii) an ability of the user to perform the breathing exercise, using the breathing apparatus with the threshold pressure or another threshold pressure, for another predetermined number of repetitions or duration of time, (iii) an ability of the user to activate head and neck muscles with a muscle strength or a muscle tension required to perform the breathing exercise using the breathing apparatus or a different breathing apparatus, or (iv) the threshold pressure of the breathing apparatus.

24. The method of claim 20, wherein the modifying the threshold pressure is based on (i) an ability of the user to perform the breathing exercise, using the breathing apparatus with the threshold pressure or another threshold pressure, for the predetermined number of repetitions or duration of time, (ii) an ability of the user to perform the breathing exercise, using the breathing apparatus with the threshold pressure or another threshold pressure, for another predetermined number of repetitions or duration of time, (iii) an ability of the user to activate head and neck muscles with a muscle strength or a muscle tension required to perform the breathing exercise using the breathing apparatus or a different breathing apparatus, or (iv) the threshold pressure of the breathing apparatus.

25. The method of claim 21, wherein the modifying the threshold pressure is based on (i) an ability of the user to perform the breathing exercise, using the another breathing apparatus with the threshold pressure or another threshold pressure, for the predetermined number of repetitions or duration of time, (ii) an ability of the user to perform the breathing exercise, using the another breathing apparatus with the threshold pressure or another threshold pressure, for another predetermined number of repetitions or duration of time, (iii) an ability of the user to activate head and neck muscles with a muscle strength or a muscle tension required to perform the breathing exercise using the another breathing apparatus or a different breathing apparatus, or (iv) the threshold pressure of the another breathing apparatus.

26. The method of claim 22, wherein the modifying the threshold pressure is based on (i) an ability of the user to perform the breathing exercise, using the another breathing apparatus with the threshold pressure or another threshold pressure, for the predetermined number of repetitions or duration of time, (ii) an ability of the user to perform the breathing exercise, using the another breathing apparatus with the threshold pressure or another threshold pressure, for another predetermined number of repetitions or duration of time, (iii) an ability of the user to activate head and neck muscles with a muscle strength or a muscle tension required to perform the breathing exercise using the another breathing apparatus or a different breathing apparatus, or (iv) the threshold pressure of the another breathing apparatus.

27. The method of claim 3, wherein the breathing apparatus and the another breathing apparatus define a combined housing.

28. The method of claim 4, wherein the breathing apparatus and the another breathing apparatus define a combined housing.

29. The method of claim 5, wherein the breathing apparatus and the another breathing apparatus define a combined housing.

30. The method of claim 6, wherein the breathing apparatus and the another breathing apparatus define a combined housing.
